(19) United States
(12) Patent Application Publication
(10) Pub. No.: US 2005/0176057 A1
(43) Pub. Date: Aug. 11, 2005

(54) Diagnostic markers of mood disorders and methods of use thereof

(76) Inventors: Troy Bremer, Dana Point, CA (US); Cornelius Diamond, San Diego, CA (US)

Correspondence Address: William C. Fuess;Suite 2G, 10951 Sorrento Valley Road, San Diego, CA, 92121-1613, US

(21) Appl. No.: 11/080,218

(22) Filed: Mar. 14, 2005

Related U.S. Application Data
(63) Continuation-in-part of application No. 10/951,085, filed on Sep. 26, 2004.
(60) Provisional application No. 60/506,253, filed on Sep. 26, 2003.

(51) Int. Cl. C12Q 1/68; A61K 31/551; G06F 19/00; G01N 33/48; G01N 33/50

(52) U.S. Cl. 435/6; 702/20; 514/220

(57) ABSTRACT

The present invention relates to methods for the diagnosis, evaluation, and treatment of mood disorders, particularly bipolar disorder. In particular, patient test samples are analyzed for the presence and amount of members of a panel of biallelic markers comprising one or more specific markers for bipolar treatment and one or more non-specific markers for bipolar treatment. A variety of markers are disclosed for assembling a panel of markers for such diagnosis and evaluation. Algorithms for determining proper treatment are disclosed. A diagnostic kit for a panel of said markers is disclosed. In various aspects, the invention provides methods for the early detection and differentiation of mood disorders or bipolar treatment. Methods for screening therapeutic compounds for mood disorders are disclosed. The invention (1) gives methods providing rapid, sensitive and specific assays that can greatly increase the number of patients that can receive beneficial treatment and therapy, thereby reducing the costs associated with incorrect diagnosis, and (2) provides methods for improved therapies.

### RELATED APPLICATIONS

The present application is descended from, and claims benefit of priority of, U.S. provisional patent application No. 60/488,137. The present application is a continuation-in-part of U.S. utility patent application Ser. No. 10/951,085, which application is itself descended from U.S. provisional patent application 60/506,253. The contents of both predecessor applications are hereby incorporated herein in their entirety, including all tables, figures, and claims

### FIELD OF THE INVENTION

The present invention generally relates to the identification and use of diagnostic markers for mood disorders, and to treatments for and response to such mood disorders. In a various aspects, the present invention particularly relates to methods for (1) the detection of and sub-classification of mood disorders, particularly bipolar disorder; (2) determining of an appropriate response(s) to treatment for such mood disorders, particularly (3) the identification of individuals at risk for post-traumatic stress disorder, rapid cycling, suicide, mixed mania and euphoric mania.

### BACKGROUND OF THE INVENTION

The following discussion of the background of the invention is merely provided to aid the reader in understanding the invention and is not admitted to describe or constitute prior art to the present invention.

Bipolar disorder, or manic depression, is a serious brain disorder that affects approximately 1-3% of the population (Daly I. (1997) Mania. Lancet 349(9059): 1157-60.). The disorder is characterized by episodes of mania and depression that can last from days to months with recurring episodes that often begin in adolescence or early adulthood. It generally requires ongoing treatment. The disease has a cost to society estimated at $32.5 billion per year in the US (Neuroscience Sf. (1997) Brain Facts.).

While there is no cure for bipolar disorder, it is a highly treatable and manageable illness. After an accurate diagnosis, most people (80 to 90 percent) can achieve substantial stabilization of their mood swings and related symptoms with proper treatment (see, for instance, Sachs G S, Printz D J, Kahn D A, Carpenter D, Docherty J P. (2000) The Expert Consensus Guideline Series: Medication Treatment of Bipolar Disorder 2000. Postgrad Med Spec No(1-104.; Sachs G S, Thase M E. (2000) Bipolar disorder therapeutics: maintenance treatment. Biol Psychiatry 48(6): 573-81.; and Huxley N A, Parikh S V, Baldessarini R J. (2000) Effectiveness of psychosocial treatments in bipolar disorder: state of the evidence. Harv Rev Psychiatry 8(3): 126-40.). Medications known as mood stabilizers are usually prescribed to help control bipolar disorder (6 Sachs G S, Printz D J, Kahn D A, Carpenter D, Docherty J P. (2000) The Expert Consensus Guideline Series: Medication Treatment of Bipolar Disorder 2000. Postgrad Med Spec No(1-104.)). Several different types of mood stabilizers are available. These include lithium, valproate, carbamazepine and olanzapine. While each has demonstrated efficacy in symptomatic treatment, lithium has remained a first-tier treatment option for the disorder (see for instance Cade J F. (2000) Lithium salts in the treatment of psychotic excitement. 1949. Bull World Health Organ 78(4): 518-20.; and Strakowski S M, Del Bello M P, Adler C M. (2001) Comparative efficacy and tolerability of drug treatments for bipolar disorder. CNS Drugs 15(9): 701-18.).

Although many believe lithium (LI) remains the treatment of choice for bipolar disorder, only 60-80% of classic bipolar patients have a satisfactory clinical response to lithium. Fewer than half of all bipolar patients have classical, elated syndromes. When the response rate of LI is considered across the wider spectrum of bipolar disorders permitted by the most recent edition of the Diagnostic and Statistical Manual of Mental Disorders (Diagnostic and Statistical Manual of Mental Disorders 1995), less than 50% of patients respond favorably to it (Gitlin et al 1995, Swann et al 1997, Vestergaard et al 1998). Among mixed manics who account for 16 to 67% of all bipolar patients, only 30-40% respond to LI (Calabrese et al 1993 a). Rapid cycling patients who constitute 13-20% of all bipolar disorders also have low (20-30%) response rates to LI (Bauer et al 1994, Dunner and Fieve 1974, Kukopulos et al 1980). Although there are numerous other predictors of LI non-response, these two variants appear to account for the largest proportion of those who exhibit poor response to LI.

Several recent naturalistic studies suggest that at present a substantial number of bipolar patients have inadequate responses to LI. Harrow and colleagues reported that 40 percent of LI-treated patients had a manic relapse during a mean 1.7 year period of treatment, a result similar to that of patients on no medication (Harrow et al 1990). Coryell reported that whereas lithium treatment yielded fewer relapses than no medication for the first 32 months of prophylaxis, subsequent relapse rates did not differ with or without lithium (Coryell et al 1997). Gitlin and colleagues reported that 17% of bipolar patients had good outcomes over 2 to 5 years. Average mood symptomatology was much more strongly associated with occupational, social and family disruption than were relapses (Gitlin et al 1995). Tohen and colleagues also reported that 46 percent of patients remained stable for four years after their first episode, with outcome being unrelated to treatment (Tohen et al 1990). Other recent prospective, open, naturalistic trials also report good outcomes in approximately on-third of patients (Maj et al 1998, Vestergaard et al 1998).

Issues of dosage and approach to discontinuation of LI have been clarified by recent studies. Discontinuation of LI therapy in bipolar patients followed by a high rate of relapse, with most episodes being manic rather than depressed, is discussed by Suppes et al, 1991. The study by Suppes et al.—as well as other withdrawal study data—strongly suggests that LI's prophylactic effect is principally in reducing the frequency of manic rather than depressive episodes (Nilsson and Axelsson 1990). Furthermore, abrupt discontinuation is followed early on by a much higher rate of relapse than is discontinuation over two or more weeks (Faedda et al 1992). Relapse rates following discontinuation were 50 percent higher among bipolar I than among bipolar II patients (Baldessarini et al 1996). Among patients with two or fewer prior episodes, maintenance LI levels of from 0.8 to 1.0 mEq/l were associated with lower rates of relapse than were levels from 0.4 to 0.6 mEq/l. Relapse rates into depression did not differ between the two groups. The severity of adverse effects was greater in the higher plasma level group (Gelenberg et al 1989).

As evidence has developed that a substantial percentage fail to do well with acute or maintenance treatment with LI, and as alternative treatments for bipolar disorder have developed, it has become important to know which illness characteristics are associated with a favorable, or an unfavorable, response to LI, as well as alternative treatments. Acutely, patients with elated, less severe episodes without psychotic features, also referred to as classical mania, appear to have the best response to LI during an acute manic episode (Goodwin and Jamison 1990). Patients who move from a depressive episode into a period of euthymia, and then mania, do better than those who move directly from a depressive episode into a manic episode (Grof et al 1987). It is not well established that these differences hold equally during maintenance treatment. Patients with more lifetime illness episodes consistently have a less good long-term outcome with lithium (Gitlin et al 1995).

Several syndromal variants of bipolar disorder are associated with relatively low response rates to LI. The best studied variant is mixed mania, with more than half a dozen studies consistently indicating lower acute and chronic response rates to LI treatment (Goodwin and Jamison 1990). Rapid cycling patients also respond less well to LI (Dunner and Fieve 1976, Kukopulos et al 1980). This illness course of bipolar disorder will be defined in the DSM-IV as 4 or more depressions, hypomanias, or manias in the prior 12 month period, with episodes being demarcated by a switch to an episode of opposite polarity or by a period of remission. This phenomenon appears to be late in onset, occurs most commonly in bipolar type II females, and is not usually associated with antidepressant use (Bauer et al 1994, Dunner and Fieve 1976, Kukopulos et al 1980 ). Rapid cycling, and possibly mixed states, appear to be non-familial modifiers of state and course that transiently come and go during the natural history of bipolar disorder and its treatment (Coryell et al 1992). When these variants are present during treatment with LI therapy, prognosis worsens and the morbidity and mortality associated with the illness increases (Calabrese et al. 1993 b, Fawcett et al. 1987). Patients with bipolar disorder which appears to be secondary to other medical disorders respond poorly to lithium. Patients with comorbid substance respond relatively poorly to lithium therapy, although this may be simply because of the conservative management of their substance abuse.

Prediction of response to mood disorder medications is important to avoid both delays in receipt of adequate treatment, as well as exposure to unnecessary side effects. Current prescription methods rely primarily on trial and error, or empirical analysis, which can take up to a year with only about a 40 to 60% expectation of positive response (see for instance Calabrese J R, Fatemi S H, Kujawa M, Woyshville M J. (1996) Predictors of response to mood stabilizers. J Clin Psychopharmacol 16(2 Suppl 1): 24S-31S.) During this evaluation period, non-responders are unnecessarily exposed to the side effects of lithium (sometimes severe; lithium toxicity), as well as being subjected to delay in recovery.

Recently, attention has focused on the identification of Single Nucleotide Polymorphisms, (hereafter SNPs) as factors that specifically influence drug action or act as markers for alleles of genes that influence drug action in lithium response (see for instance F Marndani et al., Pharmacogenetics and bipolar disorder, The Pharmacogenomics Jn., 2004, Volume 4, Number 3, Pages 161-170, for a review of the state of the art as of the instant invention). However, due to reasons described below, these single SNP variants have been shown to have little or no clinically acceptable and/or statistically significant effect by themselves.

As an independent variable, either a SNP or a patient characteristic is unlikely, itself, to indicate a responder phenotype with acceptable confidence—a direct causal effect on phenotype is rare. However, understanding the complex interactions that result in a response phenotype for more than a small number of variables is not realistic without comprehensive analysis technology. The instant invention will show how to use analysis algorithms that have the ability to extract meaningful information from complex interactions occurring between multiple variables.

In recent years, the search for a single gene responsible for mood disorder has given way to the understanding that multiple gene variants, acting together with yet unknown environmental risk factors or developmental events, interact in a complex system to account for its expression phenotype. In accordance, treatments that successfully alleviate mood disorder symptoms are likely to act on multiple gene products and thus prediction of prognosis or treatment outcome will as well.

To date, SNPs and various proteins have not been used in combination as markers of mood disorders. The current state of the art is to use clinical factors as predictors of response; these include a typical symptomatology of mood disorders [see for instance Diagnostic and Statistical Manual of Mental Disorders (4th Edition). American Psychiatric Association, Washington D.C., USA (1994).] (DSM-IV) and the absence of comorbidity with other DSM-IV axis I disturbances (e.g., substance abuse or mental retardation) [see for instance Yazici 0, Kora K, Ucok A, Tunali D, Turan N: Predictors of lithium prophylaxis in bipolar patients. J. Affect. Disord. 55, 133-42(1999).]; presence of psychotic features, such as auditory or (less frequently) visual hallucinations; female sex [see for instance Viguera A C, Tondo L, Baldessarini R J: Sex differences in response to lithium treatment. m. J. Psychiatry 157, 1509-1511 (2000).]; absence of personality disorder [see for instance Grof P, Hux M, Grof E, Arato M: Prediction of response to stabilizing lithium treatment. Pharmacopsychiatria 16, 195-200 (1983).]; and early start of treatment [see for instance Franchini L, Zanardi R, Smeraldi E, Gasperini M: Early onset of lithium prophylaxis as a predictor of good long-term outcome. Eur. Arch. Psychia. Clin. Neurosci. 249, 227-230 (1999).]. Genset Incorporated has applied for a patent (U.S. patent Office Ser. No. 20030219750 filed Nov. 27, 2003) on relating (1) biallelic markers and ADRBK2, BNDF, GSK3B, GRK3, IMPA1, IMPA2, INPP1, MARCKS, NTRK2 and/or NR1I2 genes and nucleotide sequences to (2) diagnosis of schizophrenia and bipolar disorder. However, this application does not address treatment prediction, and the samples gathered were from a case-control study focusing on disease predisposition to schizophrenia or bipolar disorder and not on disease sub-classification, i.e., likelihood to develop post-traumatic stress disorder (PTSD). The instant invention will be seen not use the genes in the aforementioned application, as well as using a complex modeling process to determine diagnosis and prognosis.

For diagnosis of, or to determine treatment outcome in, a complex diseases such as mood disorder like bipolar disorder it is necessary to use multi-factorial genetic and/or proteomic markers and/or inclusive with environment and physiological variables in combination. It is of considerable importance to the betterment of healthcare that initial prescriptions for treatment of bipolar disorder are effective in treating the patient. In addition to reducing the medical cost of wasted, ineffective medication, prescription optimization will increase the patient's quality of life by shortening recovery times and avoiding potentially life-threatening side effects (i.e. lithium toxicity). The present invention will be sent to provide for methods fulfilling these needs while providing other, related, advantages. These and other aspects of the present invention will become evident upon reference to the following detailed description and attached drawings. In addition, various references are set forth below which describe in more detail certain procedures or compositions (e.g., plasmids, algorithms, etc.), and are therefore incorporated by reference in their entireties. Preferred markers of the invention can aid in the treatment, diagnosis, differentiation, and prognosis of patients with bipolar disorder, mood disorders, and post-traumatic stress syndrome. Furthermore, the methods of the present invention expedite patient recovery, and reduce medical costs from these diseases.

### SUMMARY OF THE INVENTION

In accordance with the present invention, there are provided methods and apparatus for the identification and use of a panel of markers for the diagnosis of mood disorders. More specifically, the present invention provides a method for predicting the response to the molecule lithium in a subject with bipolar disorder comprising comparing (i) a mutational burden at one or more nucleotide positions in the genes in a sample taken from the subject who has responded to the molecule lithium with (ii) the mutational burden at one or more corresponding nucleotide positions in a control sample taken from a person who has not responded to the molecule lithium, and from this comparison predicting the probability of response to lithium in a subject. In certain embodiments the mutational burden relates to a mutation in the genes at nucleotide position determined by the reference sequence number (rs #) or combinations thereof. In certain other embodiments, at least one mutation is a silent mutation, missense mutation, or combination thereof. In certain embodiments of the invention said comparison and said prediction of probability by use of an algorithm. In certain further embodiments of the invention, the presence of the mutation is detected by a technique that may include any of 1) hybridization with oligonucleotide probes, 2) a ligation reaction, 3) a polymerase chain reaction or single nucleotide primer-guided extension assay, and/or 4) variations thereof.

In another of its aspects the present invention provides a method of detecting genetic mutations which cause bipolar or indicate a predisposition to develop bipolar disorder. The method includes determining the sequence of at least of the genes from humans known to have bipolar disorder; comparing the sequence to that of the corresponding wild type genes; and identifying mutations in the humans which correlate with the presence of bipolar disorder.

In yet another of its aspects the present invention provides a method for prediction of post-traumatic stress disorder (PTSD). The method includes 1) obtaining a bodily sample from individuals containing said person’s DNA and medical history detailing whether or not the individual has experienced PTSD; 2) Genotyping said DNA sample to detect the presence or absence of specific mutations in the genes; 3) Using said specific mutations in these genes as inputs and said medical history as outputs into an algorithm that is capable of using such inputs and outputs to correlate between those individuals who have developed PTSD and those who have not developed PTSD, said correlation process also known as training; and 4) using said correlated algorithm with said specific mutations to predict whether or not an individual has an increased probability to have experienced PTSD or is likely to experience PTSD. In certain further aspects of any of the above embodiments of the invention, the presence of the mutation is detected by a technique that may be any of hybridization with oligonucleotide probes, a ligation reaction, a polymerase chain reaction or single nucleotide primer-guided extension assay, or variations thereof.

Instill yet another of its aspects the instant invention provides a method for the prediction of suicide The method includes 1) obtaining a bodily sample from individuals containing said person's DNA and medical history detailing whether or not the individual has attempted suicide; 2) genotyping said DNA sample to detect the presence or absence of specific mutations in the genes; 3) using said specific mutations in these genes as inputs and said medical history as outputs into an algorithm that is capable of using such inputs and outputs to correlate between those individuals who have attempted suicide and those who have not attempted suicide, said correlation process also known as training; and 4) using said correlated algorithm with said specific mutations to predict whether or not an individual has an increased probability to have attempted suicide or is likely to attempt suicide. In certain further embodiments of any of the above aspects of the invention, the presence of the mutation is detected by a technique that may be hybridization with oligonucleotide probes, a ligation reaction, a polymerase chain reaction or single nucleotide primer-guided extension assay, or variations thereof.

Considering still yet another aspect of the present invention, the invention provides a method for prediction of bipolar sub-type. The method includes 1) obtaining a bodily sample from individuals containing said person's DNA and medical history detailing whether or not the individual has experienced a specific type of bipolar disorder; 2) genotyping said DNA sample to detect the presence or absence of specific mutations in the genes; 3) using said specific mutations in these genes as inputs and said medical history as outputs into an algorithm that is capable of using such inputs and outputs to correlate between those individuals who have developed said specific type of bipolar disorder and those who have not developed said specific type of bipolar disorder, said correlation process also known as training; and 4) using said correlated algorithm with said specific mutations to predict whether or not an individual has an increased probability to have experienced said specific type of bipolar disorder or is likely to experience said specific type of bipolar disorder. In certain further aspects of any of the above embodiments of the invention, the presence of the mutation is detected by a technique that may be hybridization with oligonucleotide probes, a ligation reaction, a polymerase chain reaction or single nucleotide primer-guided extension assay, or variations thereof.

In still yet another of its aspect the present invention provides a method for evaluating a compound for use in diagnosis or treatment of bipolar disorder. The method includes 1) contacting a predetermined quantity of the compound with cultured cybrid cells having genomic DNA originating from a .rho..sup.0 cell line or a bipolar animal model and genes originating from tissue of a human having a disorder that is associated with bipolar disorder; 2) measuring a phenotypic trait in the cybrid cells or bipolar animal model that correlates with the presence of said human genes and that is not present in cultured cybrid cells having genomic DNA originating from a .rho..sup.0 cell line or normal animal model and said human genes originating from tissue of a human free of a disorder that is associated with bipolar disorder; and 3) correlating a change in the phenotypic trait with effectiveness of the compound.

A Method for Defining Panels of Markers

In practice of the methods of the present invention data may be obtained from a group of subjects. The subjects may be patients who have been tested for the presence or level of certain markers. Such markers and methods of patient extraction are well known to those skilled in the art. A particular set of markers may be relevant to a particular condition or disease. The method is not dependent on the actual markers. The markers discussed in this document are included only for illustration and are not intended to limit the scope of the invention. Examples of such markers and panels of markers are described in the instant invention and the incorporated references.

The collection of patient samples is well-known to one of ordinary skill in the medical arts. A preferred embodiment of the instant invention is that the samples come from two or more different sets of patients, one a disease group of interest and the other(s) a control group, which may be healthy or diseased in a different indication than the disease group of interest. For instance, one might want to look at the difference in DNA mutations between patients who have had bipolar disorder and those who had schizophrenia to differentiate between the two populations.

The DNA samples are assayed, and the resulting set of values are put into a database, along with medical, also called phenotypic, information detailing the illness type, for instance response to certain medications, once this is known. Additional clinical details such as clinical co-diagnoses, such as history of disposition to suicide or rapid cycling subtype, and patient physiological, medical, and demographics, the sum total called patient characteristics, are put into the database. The database can be simple as a spreadsheet, i.e. a two-dimensional table of values, with rows being patients and columns being filled with patient marker and other characteristic values.

From this database, a computerized algorithm first performs pre-processing of the data values. This involves normalization of the values across the dataset and/or transformation into a different representation for further processing. The dataset is then analyzed for missing values. Missing values are either replaced using an inputation algorithm, in a preferred embodiment using KNN or MVC algorithms, or the patient attached to the missing value is excised from the database. If greater than 50% of the other patients have the same missing value then value can be ignored.

Once all missing values have been accounted for, the dataset is split up into three parts: a training set comprising 33-80% of the patients and their associated values, a testing set comprising 10-50% of the patients and their associated values, and a validation set comprising 1-50% of the patients and their associated values. These datasets can be fturther sub-divided or combined according to algorithmic accuracy. A feature selection algorithm is applied to the training dataset. This feature selection algorithm selects the most relevant marker values and/or patient characteristics. Preferred feature selection algorithms include, but are not limited to, Forward or Backward Floating, SVMs, Markov Blankets, Tree Based Methods with node discarding, Genetic Algorithms, Regression-based methods, kernel-based methods, and filter-based methods.

Feature selection is done in a cross-validated fashion, preferably in a naive or k-fold fashion, as to not induce bias in the results and is tested with the testing dataset. Cross-validation is one of several approaches to estimating how well the features selected from some training data is going to perform on future as-yet-unseen data and is well-known to the skilled artisan. Cross validation is a model evaluation method that is better than residuals. The problem with residual evaluations is that they do not give an indication of how well the learner will do when it is asked to make new predictions for data it has not already seen. One way to overcome this problem is to not use the entire data set when training a learner. Some of the data is removed before training begins. Then when training is done, the data that was removed can be used to test the performance of the learned model on “new” data.

Once the algorithm has returned a list of selected markers, one can optimize these selected markers by applying a classifer to the training dataset to predict clinical outcome. A cost function that the classifier optimizes is specified according to outcome desired, for instance an area under receiver-operator curve maximizing the product of sensitivity and specificity of the selected markers, or positive or negative predictive accuracy. Testing of the classifier is done on the testing dataset in a cross-validated fashion, preferably naive or k-fold cross-validation. Further detail is given in U.S. patent application Ser. No. 09/611,220, incorporated by reference. Classifiers map input variables, in this case patient marker values, to outcomes of interest, for instance, prediction of stroke sub-type. Preferred classifiers include, but are not limited to, neural networks, Decision Trees, genetic algorithms, SVMs, Regression Trees, Cascade Correlation, Group Method Data Handling (GMDH), Multivariate Adaptive Regression Splines (MARS), Multilinear Interpolation, Radial Basis Functions, Robust Regression, Cascade Correlation+Projection Pursuit, linear regression, Non-linear regression, Polynomial Regression, Regression Trees, Multilinear Interpolation, MARS, Bayes classifiers and networks, and Markov Models, and Kernel Methods.

The classification model is then optimized by for instance combining the model with other models in an ensemble fashion. Preferred methods for classifier optimization include, but are not limited to, boosting, bagging, entropy-based, and voting networks. This classifier is now known as the final predictive model. The predictive model is tested on the validation data set, not used in either feature selection or classification, to obtain an estimate of performance in a similar population.

The predictive model can be translated into a decision tree format for subdividing the patient population and making the decision output of the model easy to understand for the clinician. The marker input values might include a time since symptom onset value and/or a threshold value. Using these marker inputs, the predictive model delivers diagnostic or prognostic output value along with associated error. The instant invention anticipates a kit comprised of reagents, devices and instructions for performing the assays, and a computer software program comprised of the predictive model that interprets the assay values when entered into the predictive model run on a computer. The predictive model receives the marker values via the computer that it resides upon.

Once patients are exhibiting symptoms of mood disorders, for instance bipolar disorder, a DNA sample, from blood draw or buccal swab, for instance, is obtained from the patient using standard techniques well known to those of ordinary skill in the art and assayed for various DNA markers of mood disorders. Assays can be preformed through nucleic acid tests or through any of the other techniques well known to the skilled artisan. In a preferred embodiment, the assay is in a format that permits multiple markers to be tested from one sample, such as the Luminex platform.™ and/or in a rapid fashion, defined to be under 30 minutes and in the most preferred enablement of the instant invention, under 15 minutes. The values of the markers in the samples are received into the trained, tested, and validated algorithm residing on a computer, which outputs to the user on a display and/or in printed format on paper and/or transmits the information to another display source the result of the algorithm calculations in numerical form, a probability estimate of the clinical diagnosis of the patient. There is an error given to the probability estimate, in a preferred embodiment this error level is a confidence level. The medical worker can then use this diagnosis to help guide treatment of the patient.

In another embodiment, the present invention provides a kit for the analysis of markers. Said diagnostic kit includes one or more polynucleotides of the invention, optionally with a portion or all of the necessary reagents, software-based computer algorithms and instructions for genotyping a test subject by determining the identity of a nucleotide at biallelic markers of the invention. The polynucleotides of a kit may optionally be attached to a solid support, or be part of an array or addressable array of polynucleotides. The kit may provide for the determination of the identity of the nucleotide at a marker position by any method known in the art including, but not limited to, a sequencing assay method, a microsequencing assay method, a hybridization assay method, or enzyme-based mismatch detection assay. Optionally such a kit may include instructions for scoring the results of the determination with respect to the test subjects' predisposition to bipolar disorder, or likely response to an agent acting on bipolar disorder, or chances of suffering from side effects to an agent acting on bipolar disorder. Optionally the kits may contain one or more means for using information obtained from said performed nucleic acid tests to rule in or out certain diagnoses.

These and other aspects of the present invention will become evident upon reference to the following detailed description and attached drawings. In addition, various references are set forth herein which describe in more detail certain aspects of this invention, and are therefore incorporated by reference in their entireties.

### BRIEF DESCRIPTION OF THE DRAWINGS

FIG. 1 is a set of tables detailing SNPs included in the machine learning model for lithium response developed for patients with no history of suicidal ideation, and performance of this model on a training set, with 50× cross-validation, and for indicated and non-indicated populations;

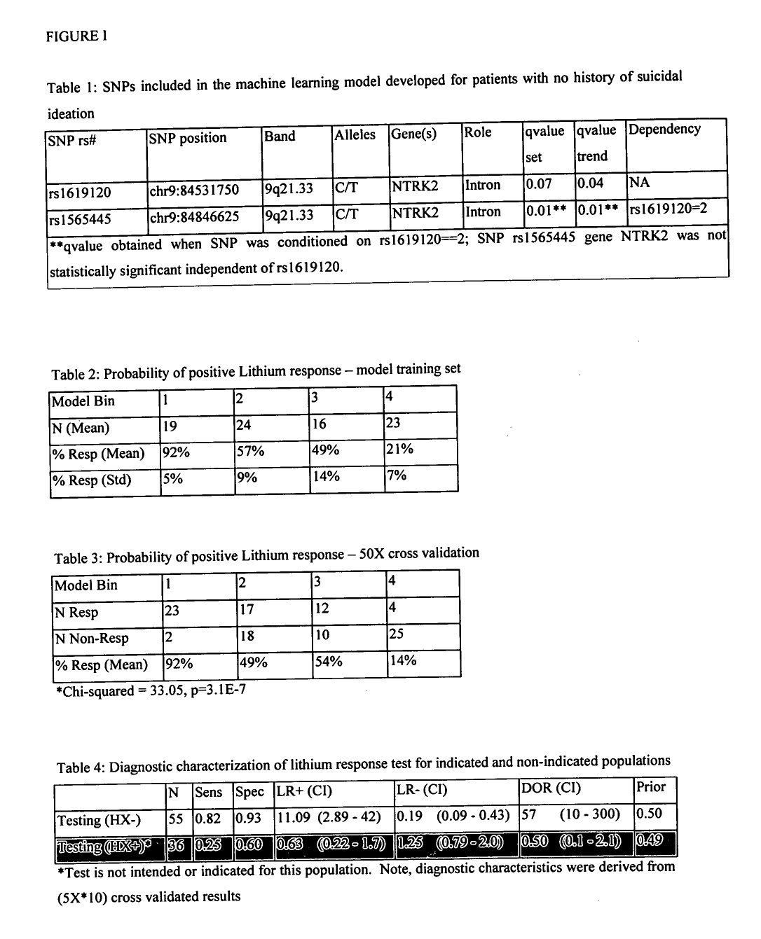

FIG. 2 is a set of tables detailing SNPs included in the machine learning model for lithium response developed for patients with no history of suicidal ideation, performance of this model on the non-indicated population, significant univariate SNPs in these patients, and associations between SNPs that are associated with lithium response in NTRK2;

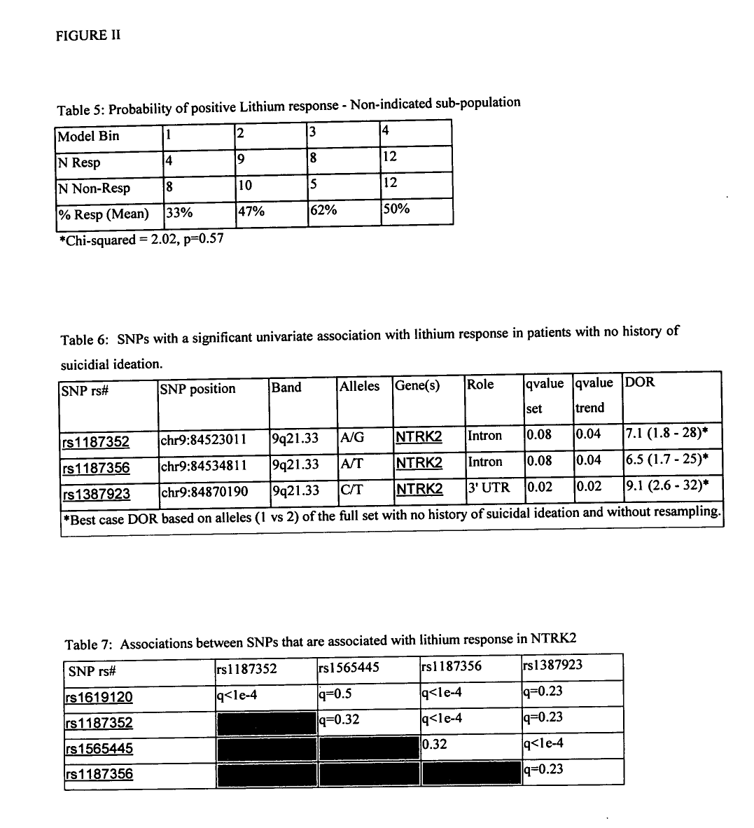

FIG. 3 is a set of tables detailing SNPs included in another machine learning model for lithium response developed for patients with no history of suicidal ideation, and performance of this model on a training set, with 50× cross-validation, for indicated and non-indicated populations;

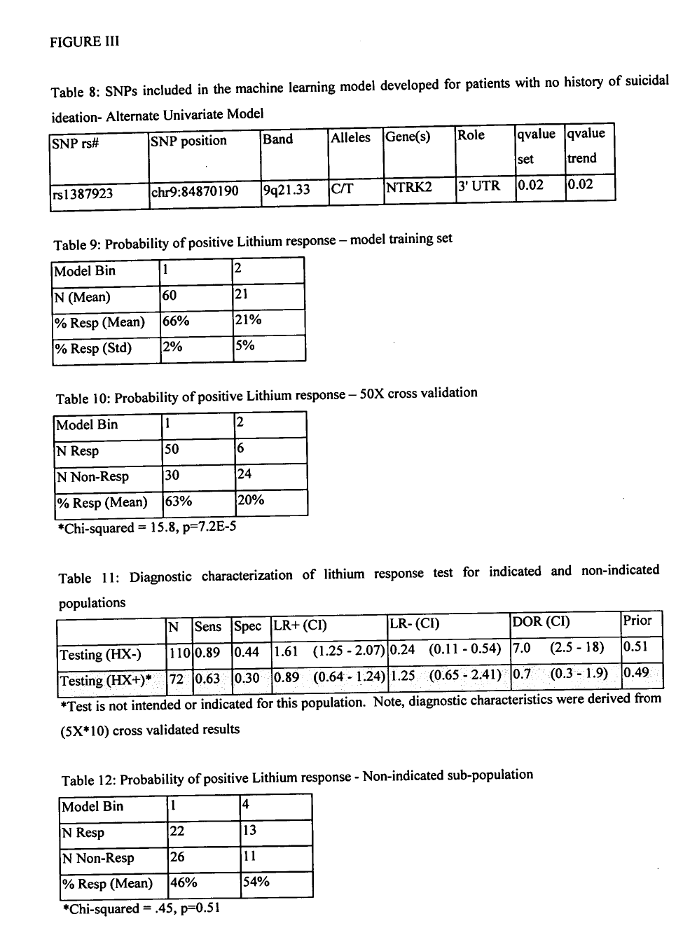

FIG. 4 is a set of tables detailing SNPs included in the machine learning model for lithium response developed for patients with a co-diagnosis of panic disorder, and performance of this model on a training set, with 50× cross-validation, and for indicated and non-indicated populations;

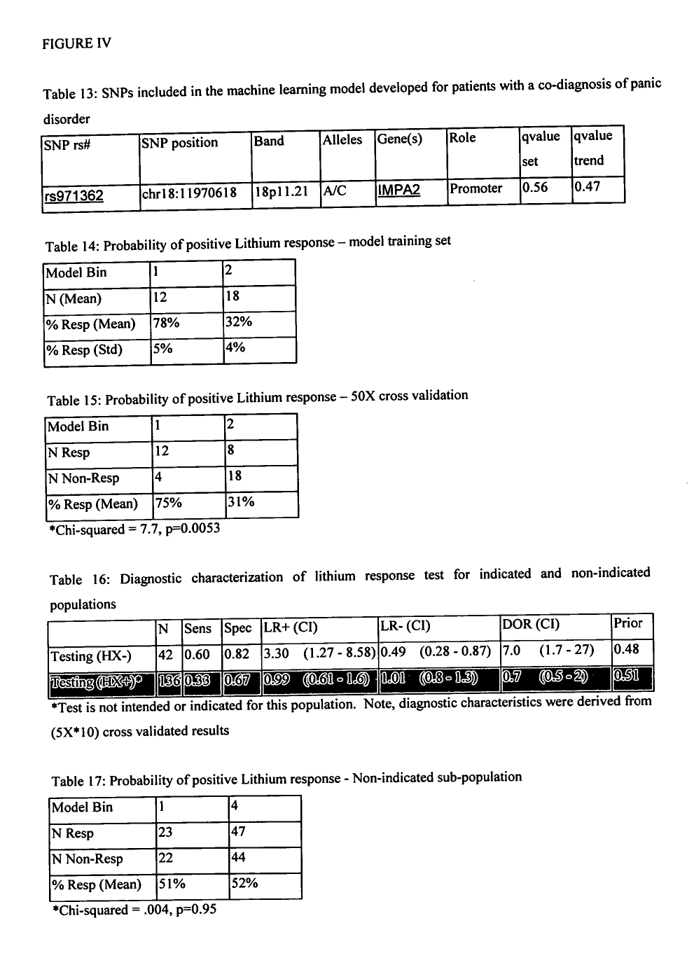

FIG. 5 is a set of tables detailing SNPs included in the machine learning model for lithium response developed for patients with a negative co-diagnosis of rapid cycling, and performance of this model on a training set, with 50× cross-validation, and for indicated and non-indicated populations;

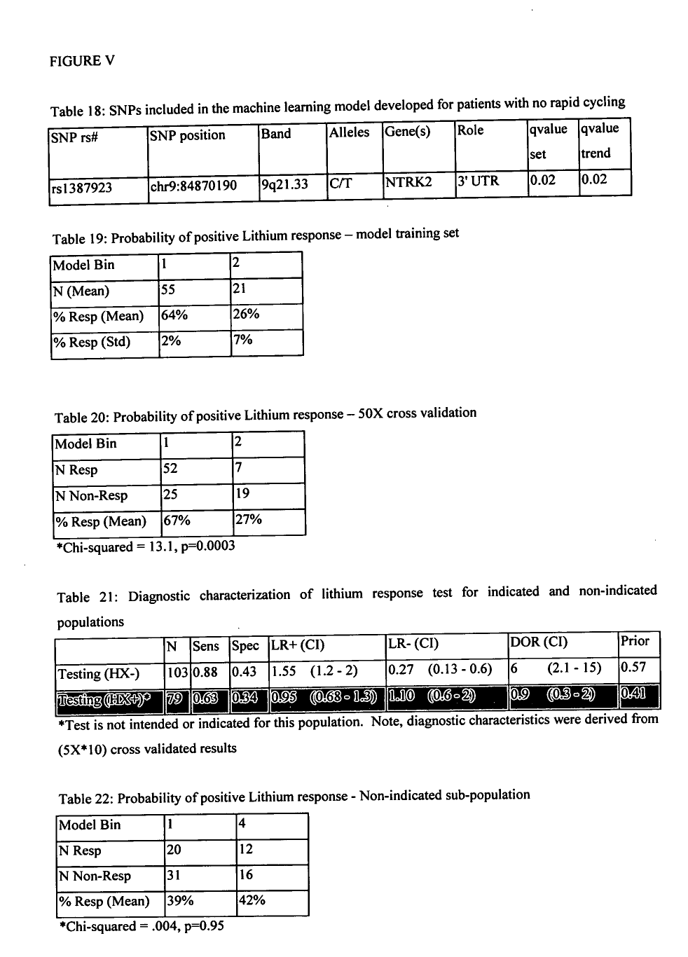

FIG. 6 is a table detailing SNPs included in the machine learning model for lithium response developed for patients with a negative history/co-diagnosis of rapid cycling, and probability of positive Lithium response—Rapid Cycling vs Non-Rapid Cycling with SNP rs1387923 of gene NTRK2;

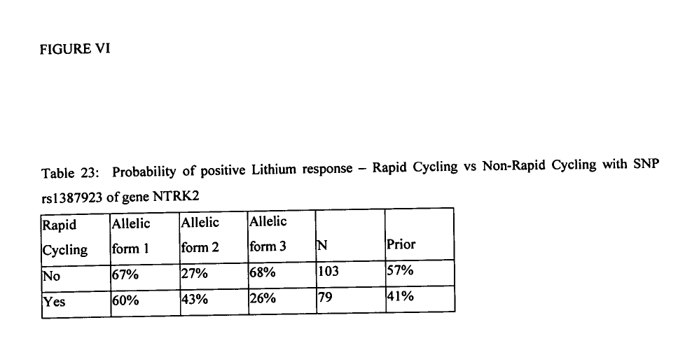

FIG. 7 is a set of tables detailing SNPs included in the machine learning model for lithium response developed for patients with a negative co-diagnosis/history of Dysphoric Mania/Mixed States, and performance of this model on a training set, with 50x cross-validation, and for indicated and non-indicated populations;

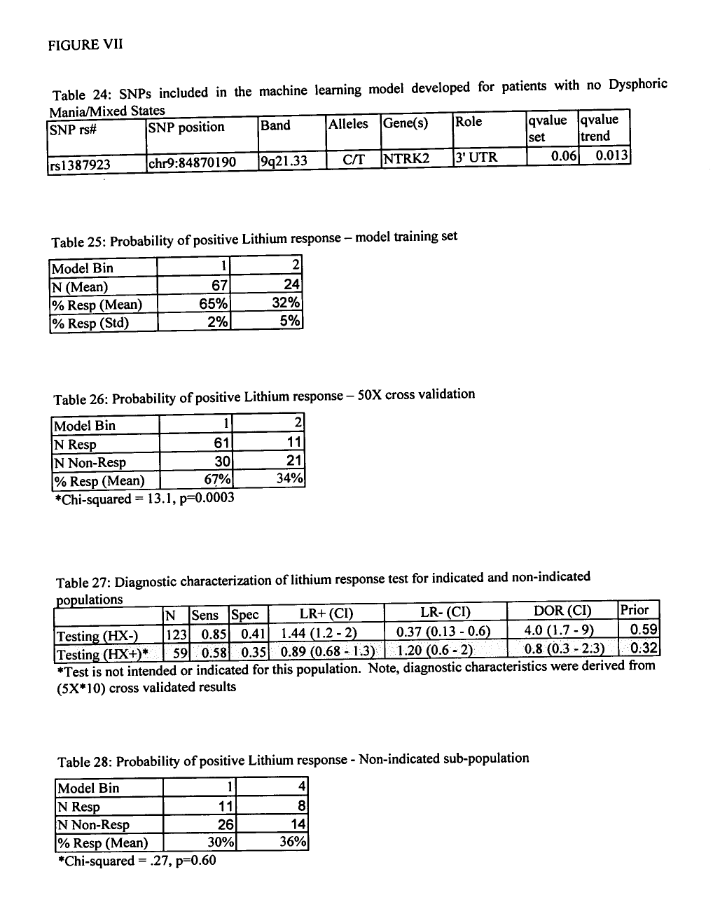

FIG. 8 is a set of tables detailing SNPs included in the machine learning model-for lithium response developed for patients with a negative co-diagnosis of panic disorder, and performance of this model on a training set, with 50× cross-validation, and for indicated and non-indicated populations;

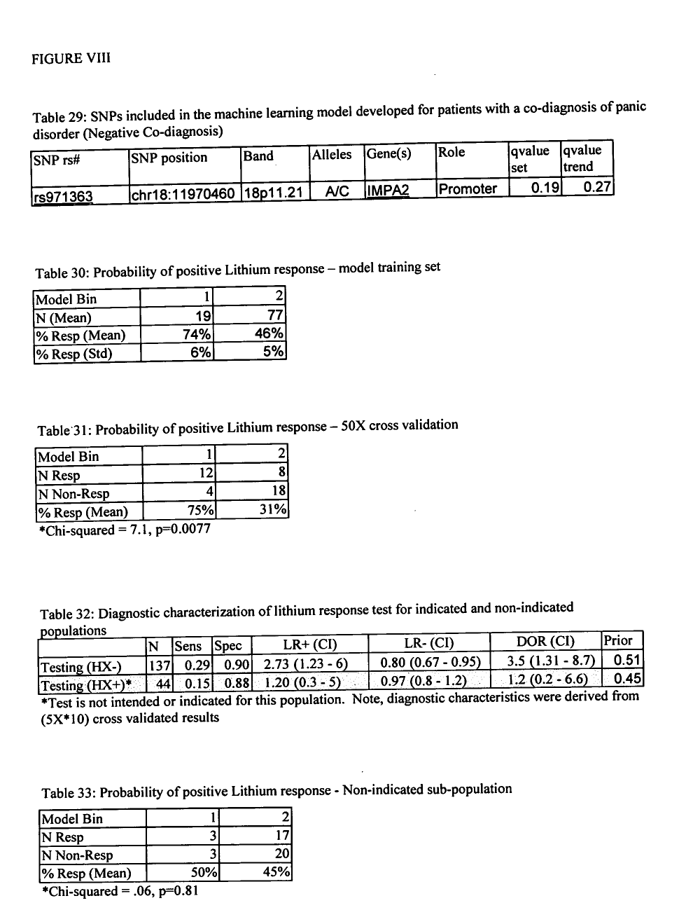

FIG. 9 is a set of tables detailing SNPs included in the combined machine learning model for lithium response developed for patients with a positive or negative co-diagnosis of panic disorder, and performance of this model on a training set, with 50× cross-validation, and for indicated and non-indicated populations;

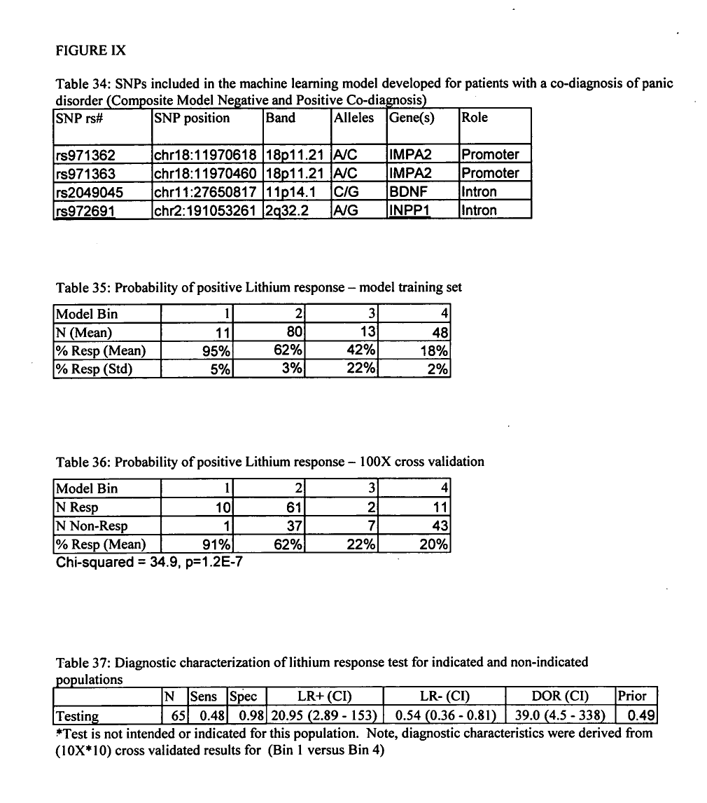

FIG. 10 is a set of tables detailing SNPs included in the combined machine learning model for lithium response developed for patients with a negative co-diagnosis of post-traumatic stress disorder, and performance of this model on a training set, with 50× cross-validation, and for indicated and non-indicated populations;

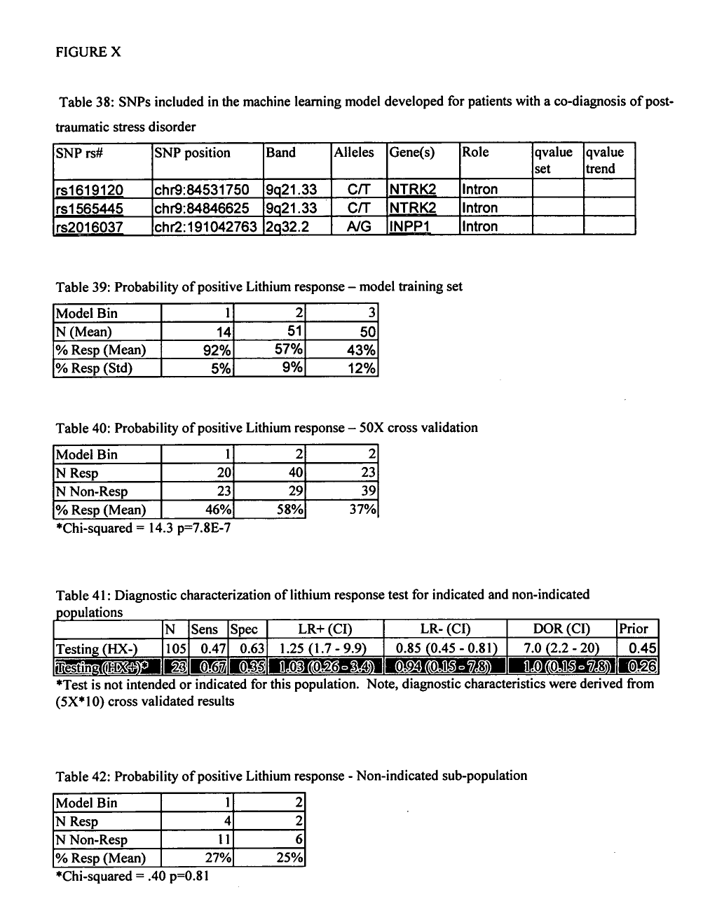

FIG. 11 is a set of tables detailing scoring for two decision tree models, the probability of positive Lithium response on a model training set and with 100× cross validation using a composite model, the diagnostic characterization of this lithium response test composite model for BIN 1+2 patients vs BIN 4 patients and for BIN 1+2 patients vs 3+4 patients, and the genes and SNPs included in this composite model;

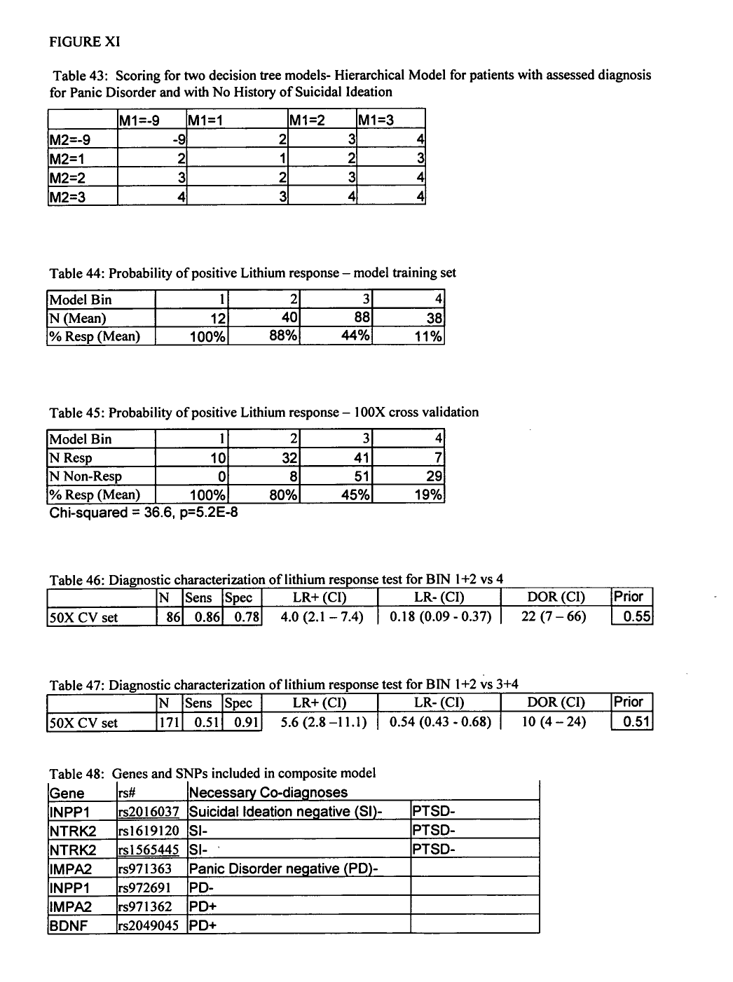

FIG. 12 is a set of tables detailing scoring for a general hierarchical model, the probability of positive Lithium response on with 50× cross validation using a prior knowledge composite model, the diagnostic characterization of this lithium response test prior knowledge composite model for BIN 1+2 patients vs BIN 3.5-4 patients and for BIN 1+2 patients vs 4 patients, and the genes and SNPs included in this prior knowledge composite model.

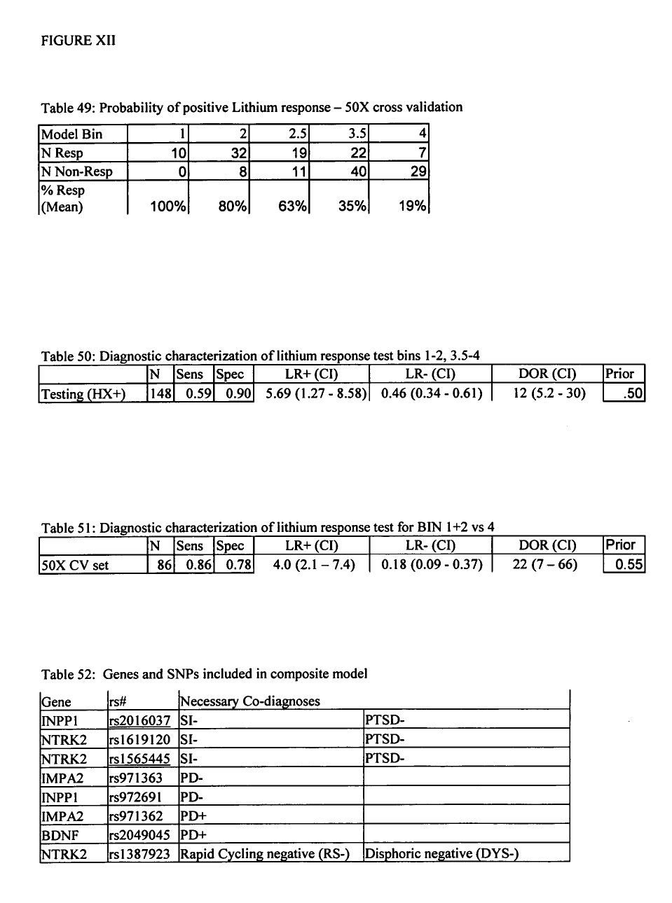

### DETAILED DESCRIPTION OF THE INVENTION

It is important to an understanding of the present invention to note that all technical and scientific terms used herein, unless otherwise defined, are intended to have the same meaning as commonly understood by one of ordinary skill in the art. The techniques employed herein are also those that are known to one of ordinary skill in the art, unless stated otherwise.

### DEFINITIONS

As used interchangeably herein, the term “oligonucleotides”, and “polynucleotides” include RNA, DNA, or RNA/DNA hybrid sequences of more than one nucleotide in either single chain or duplex form. The term “nucleotide” as used herein as an adjective to describe molecules comprising RNA, DNA, or RNA/DNA hybrid sequences of any length in single-stranded or duplex form. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. Although the term “nucleotide” is also used herein to encompass “modified nucleotides” which comprise at least one modifications (a) an alternative linking group, (b) an analogous form of purine, (c) an analogous form of pyrimidine, or (d) an analogous sugar, for examples of analogous linking groups, purine, pyrimidines, and sugars see for example PCT publication No. WO 95/04064, the disclosure of which is incorporated herein by reference. However, the polynucleotides of the invention are preferably comprised of greater than 50% conventional deoxyribose nucleotides, and most preferably greater than 90% conventional deoxyribose nucleotides. The polynucleotide sequences of the invention may be prepared by any known method, including synthetic, recombinant, ex vivo generation, or a combination thereof, as well as utilizing any purification methods known in the art.

The term “purified” is used herein to describe a polynucleotide or polynucleotide vector of the invention which has been separated from other compounds including, but not limited to other nucleic acids, carbohydrates, lipids and proteins (such as the enzymes used in the synthesis of the polynucleotide), or the separation of covalently closed polynucleotides from linear polynucleotides. A polynucleotide is substantially pure when at least about 50%, preferably 60 to 75% of a sample exhibits a single polynucleotide sequence and conformation (linear versus covalently close). A substantially pure polynucleotide typically comprises about 50%, preferably 60 to 90% weight/weight of a nucleic acid sample, more usually about 95%, and preferably is over about 99% pure. Polynucleotide purity or homogeneity may be indicated by a number of means well known in the art, such as agarose or polyacrylamide gel electrophoresis of a sample, followed by visualizing a single polynucleotide band upon staining the gel. For certain purposes higher resolution can be provided by using HPLC or other means well known in the art.

The term “isolated” requires that the material be removed from its original environment (e.g., the natural environment if it is naturally occurring). For example, a naturally-occurring polynucleotide or polypeptide present in a living animal is not isolated, but the same polynucleotide or DNA or polypeptide, separated from some or all of the coexisting materials in the natural system, is isolated. Such polynucleotide could be part of a vector and/or such polynucleotide or polypeptide could be part of a composition, and still be isolated in that the vector or composition is not part of its natural environment.

The term “primer” denotes a specific oligonucleotide sequence which is complementary to a target nucleotide sequence and used to hybridize to the target nucleotide sequence. A primer serves as an initiation point for nucleotide polymerization catalyzed by either DNA polymerase, RNA polymerase or reverse transcriptase.

The term “probe” denotes a defined nucleic acid segment (or nucleotide analog segment, e.g., polynucleotide as defined herein) which can be used to identify a specific polynucleotide sequence present in samples, said nucleic acid segment comprising a nucleotide sequence complementary of the specific polynucleotide sequence to be identified.

The terms “trait” and “phenotype” are used interchangeably herein and refer to any clinically distinguishable, detectable or otherwise measurable property of an organism such as symptoms of, or susceptibility to a disease for example. Typically the terms “trait” or “phenotype” are used herein to refer to symptoms of, or susceptibility to schizophrenia or bipolar disorder; or to refer to an individual's response to an agent acting on schizophrenia or bipolar disorder; or to refer to symptoms of, or susceptibility to side effects to an agent acting on schizophrenia or bipolar disorder.

The term “allele” is used herein to refer to variants of a nucleotide sequence. A biallelic polymorphism has two forms. Typically the first identified allele is designated as the original allele whereas other alleles are designated as alternative alleles. Diploid organisms may be homozygous or heterozygous for an allelic form. Biallelic markers generally comprise a polymorphism at one single base position. Each biallelic marker therefore corresponds to two forms of a polynucleotide sequence which, when compared with one another, present a nucleotide modification at one position. Usually, the nucleotide modification involves the substitution of one nucleotide for another. Optionally allele I or allele 2 of the biallelic markers disclosed in APPENDIX I may be specified as being present at the biallelic marker of the invention. The contiguous span may optionally include a nucleotide at a polymorphism position described in APPENDIX I, including single nucleotide substitutions, deletions as well as multiple nucleotide deletions.

The term “heteroygosity rate” is used herein to refer to the incidence of individuals in a population, which are heterozygous at a particular allele. In a biallelic system the heterozygosity rate is on average equal to 2P.sub.a(1-P.sub.a), where P.sub.a is the frequency of the least common allele. In order to be useful in genetic studies a genetic marker should have an adequate level of heterozygosity to allow a reasonable probability that a randomly selected person will be heterozygous.

The term “genotype” as used herein refers the identity of the alleles present in an individual or a sample. In the context of the present invention a genotype preferably refers to the description of the biallelic marker alleles present in an individual or a sample. The term “genotyping” a sample or an individual for a biallelic marker involves determining the specific allele or the specific nucleotide(s) carried by an individual at a biallelic marker.

The term “mutation” as used herein refers to a difference in DNA sequence between or among different genomes or individuals which has a frequency at or above 1%.

The term “haplotype” refers to a combination of alleles present in an individual or a sample on a single chromosome. In the context of the present invention a haplotype preferably refers to a combination of biallelic marker alleles found in a given individual and which may be associated with a phenotype.

The term “polymorphism” as used herein refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. A polymorphism may comprise a substitution, deletion or insertion of one or more nucleotides. A single nucleotide polymorphism is a single base pair change. Typically a single nucleotide polymorphism is the replacement of one nucleotide by another nucleotide at the polymorphic site. Deletion of a single nucleotide or insertion of a single nucleotide, also give rise to single nucleotide polymorphisms. Typically, between different genomes or between different individuals, the polymorphic site may be occupied by two different nucleotides.

The terms “biallelic polymorphism”, “SNP” and “biallelic marker” are used interchangeably herein to refer to a polymorphism having two alleles at a fairly high frequency in the population, preferably a single nucleotide polymorphism. A “biallelic marker allele” refers to the nucleotide variants present at a biallelic marker site. Typically the frequency of the less common allele of the biallelic markers of the present invention has been validated to be greater than 1%, preferably the frequency is greater than 10%, more preferably the frequency is at least 20% (i.e. heterozygosity rate of at least 0.32), even more preferably the frequency is at least 30% (i.e. heterozygosity rate of at least 0.42). A biallelic marker wherein the frequency of the less common allele is 30% or more is termed a “high quality biallelic marker.” All of the genotyping, haplotyping, association, and interaction study methods of the invention may optionally be performed solely with high quality biallelic markers.

The location of nucleotides in a polynucleotide with respect to the center of the polynucleotide are described herein in the following manner. When a polynucleotide has an odd number of nucleotides, the nucleotide at an equal distance from the 3′ and 5′ ends of the polynucleotide is considered to be “at the center” of the polynucleotide, and any nucleotide immediately adjacent to the nucleotide at the center, or the nucleotide at the center itself is considered to be “within I nucleotide of the center.” With an odd number of nucleotides in a polynucleotide any of the five nucleotides positions in the middle of the polynucleotide would be considered to be within 2 nucleotides of the center, and so on. When a polynucleotide has an even number of nucleotides, there would be a bond and not a nucleotide at the center of the polynucleotide. Thus, either of the two central nucleotides would be considered to be “within I nucleotide of the center” and any of the four nucleotides in the middle of the polynucleotide would be considered to be “within 2 nucleotides of the center”, and so on. For polymorphisms which involve the substitution, insertion or deletion of I or more nucleotides, the polymorphism, allele or biallelic marker is “at the center” of a polynucleotide if the difference between the distance from the substituted, inserted, or deleted polynucleotides of the polymorphism and the 3′ end of the polynucleotide, and the distance from the substituted, inserted, or deleted polynucleotides of the polymorphism and the 5′ end of the polynucleotide is zero or one nucleotide. If this difference is 0 to 3, then the polymorphism is considered to be “within I nucleotide of the center.” If the difference is 0 to 5, the polymorphism is considered to be “within 2 nucleotides of the center.” If the difference is 0 to 7, the polymorphism is considered to be “within 3 nucleotides of the center,” and so on. For polymorphisms which involve the substitution, insertion or deletion of I or more nucleotides, the polymorphism, allele or biallelic marker is “at the center” of a polynucleotide if the difference between the distance from the substituted, inserted, or deleted polynucleotides of the polymorphism and the 3′ end of the polynucleotide, and the distance from the substituted, inserted, or deleted polynucleotides of the polymorphism and the 5′ end of the polynucleotide is zero or one nucleotide. If this difference is 0 to 3, then the polymorphism is considered to be “within 1 nucleotide of the center.” If the difference is 0 to 5, the polymorphism is considered to be “within 2 nucleotides of the center.” If the difference is 0 to 7, the polymorphism is considered to be “within 3 nucleotides of the center,” and so on.

The term “upstream” is used herein to refer to a location which, is toward the 5′ end of the polynucleotide from a specific reference point.

The terms “base paired” and “Watson & Crick base paired” are used interchangeably herein to refer to nucleotides which can be hydrogen bonded to one another be virtue of their sequence identities in a manner like that found in double-helical DNA with thymine or uracil residues linked to adenine residues by two hydrogen bonds and cytosine and guanine residues linked by three hydrogen bonds (See Stryer, L., Biochemistry, 4th edition, 1995).

The terms “complementary” or “complement thereof” are used herein to refer to the sequences of polynucleotides which is capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind.

The terms “ADRBK2 gene”, when used herein, encompasses genomic, mRNA and cDNA sequences encoding the ADRBK2 polypeptide, including the untranslated regulatory, promoter, and intronic regions of the genomic DNA.

The terms “BDNF gene”, when used herein, encompasses genomic, mRNA and cDNA sequences encoding the BDNF protein, including the untranslated regulatory, promoter, and intronic regions of the genomic DNA.

The terms “GSK3B gene”, when used herein, encompasses genomic, mRNA and cDNA sequences encoding the GSK3B protein, including the untranslated regulatory, promoter, and intronic regions of the genomic DNA.

The terms “IMPA1 gene”, when used herein, encompasses genomic, mRNA and cDNA sequences encoding the IMPA1 protein, including the untranslated regulatory, promoter, and intronic regions of the genomic DNA.

The terms “IMPA2 gene”, when used herein, encompasses genomic, mRNA and cDNA sequences encoding the IMPA2 protein, including the untranslated regulatory, promoter, and intronic regions of the genomic DNA.

The terms “INPP1 gene”, when used herein, encompasses genomic, mRNA and cDNA sequences encoding the INPP1 protein, including the untranslated regulatory, promoter, and intronic regions of the genomic DNA.

The terms “MARCKS gene”, when used herein, encompasses genomic, mRNA and cDNA sequences encoding the MARCKS protein, including the untranslated regulatory, promoter, and intronic regions of the genomic DNA.

The terms “NR1I2 gene”, when used herein, encompasses genomic, mRNA and cDNA sequences encoding the NR1I2 protein, including the untranslated regulatory, promoter, and intronic regions of the genomic DNA.

The terms “NTKR2 gene”, when used herein, encompasses genomic, mRNA and cDNA sequences encoding the NTKR2 protein, including the untranslated regulatory, promoter, and intronic regions of the genomic DNA.

As used herein the term “GRK-related biallelic marker” relates to a set of biallelic markers residing in the human chromosome 22q11.23 region. The term GRK-related biallelic marker encompasses all of the biallelic markers disclosed in APPENDIX I and any biallelic markers in linkage disequilibrium therewith. The preferred chromosome 22q11.23-related biallelic marker alleles of the present invention include each one the alleles described in APPENDIX I individually or in groups consisting of all the possible combinations of the alleles listed.

The term “polypeptide” refers to a polymer of amino acids without regard to the length of the polymer; thus, peptides, oligopeptides, and proteins are included within the definition of polypeptide. This term also does not specify or exclude prost-expression modifications of polypeptides, for example, polypeptides which include the covalent attachment of glycosyl groups, acetyl groups, phosphate groups, lipid groups and the like are expressly encompassed by the term polypeptide. Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring.

The term “purified” is used herein to describe a polypeptide of the invention which has been separated from other compounds including, but not limited to nucleic acids, lipids, carbohydrates and other proteins. A polypeptide is substantially pure when at least about 50%, preferably 60 to 75% of a sample exhibits a single polypeptide sequence. A substantially pure polypeptide typically comprises about 50%, preferably 60 to 90% weight/weight of a protein sample, more usually about 95%, and preferably is over about 99% pure. Polypeptide purity or homogeneity is indicated by a number of means well known in the art, such as agarose or polyacrylamide gel electrophoresis of a sample, followed by visualizing a single polypeptide band upon staining the gel. For certain purposes higher resolution can be provided by using HPLC or other means well known in the art.

As used herein, the term “non-human animal” refers to any non-human vertebrate, birds and more usually mammals, preferably primates, farm animals such as swine, goats, sheep, donkeys, and horses, rabbits or rodents, more preferably rats or mice. As used herein, the term “animal” is used to refer to any vertebrate, preferable a mammal. Both the terms “animal” and “mammal” expressly embrace human subjects unless preceded with the term “non-human”.

As used herein, the term “antibody” refers to a polypeptide or group of polypeptides which are comprised of at least one binding domain, where an antibody binding domain is formed from the folding of variable domains of an antibody molecule to form three-dimensional binding spaces with an internal surface shape and charge distribution complementary to the features of an antigenic determinant of an antigen., which allows an immunological reaction with the antigen. Antibodies include recombinant proteins comprising the binding domains, as wells as fragments, including Fab, Fab′, F(ab).sub.2, and F(ab′).sub.2 fragments.

As used herein, an “antigenic determinant” is the portion of an antigen molecule, that determines the specificity of the antigen-antibody reaction. An “epitope” refers to an antigenic determinant of a polypeptide. An epitope can comprise as few as 3 amino acids in a spatial conformation which is unique to the epitope. Generally an epitope comprises at least 6 such amino acids, and more usually at least 8-10 such amino acids. Methods for determining the amino acids which make up an epitope include x-ray crystallography, 2-dimensional nuclear magnetic resonance, and epitope mapping e.g. the Pepscan method described by Geysen et al. 1984; PCT Publication No. WO 84/03564; and PCT Publication No. WO 84/03506.

The terms used herein are not intended to be limiting of the invention. For example, the term “gene” includes cDNAs, RNA, or other polynucleotides that encode gene products. In using the terms “nucleic acid”, “RNA”, “DNA”, etc., there is no intention to limit the chemical structures that can be used in particular steps. For example, it is well known to those skilled in the art that RNA can generally be substituted for DNA, and as such, the use of the term “DNA” should be read to include this substitution. In addition, it is known that a variety of nucleic acid analogues and derivatives can be made and will hybridize to one another and to DNA and RNA, and the use of such analogues and derivatives is also within the scope of the present invention.

“Isolating” a substance refers to removing a material from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acids could be part of a vector and/or such nucleic acids or polypeptides could be part of a composition, and still be isolated in that such vector or composition is not part of its natural environment.

“Expression” of a gene or nucleic acid encompasses not only cellular gene expression, but also the transcription and translation of nucleic acid(s) in cloning systems and in any other context. “At least one mutation” denotes the substitution, addition or deletion of at least one nucleotide anywhere in a nucleotide sequence. “Point mutations” are mutations within a nucleotide sequence that result in a change from one nucleotide to another; “silent mutations” are mutations that do not result in a change in the amino acid sequence encoded by the nucleotide sequence. “Mutational burden” refers to any qualitative assessment or quantification of the proportion of nucleic acid molecules in a sample having at least one mutation in a region of a specific nucleic acid sequence, relative to the proportion of nucleic acid molecules having the wildtype sequence for the corresponding nucleic acid region.

Variants and Fragments

The invention also relates to variants and fragments of the polynucleotides described herein. Variants of polynucleotides, as the term is used herein, are polynucleotides that differ from a reference polynucleotide. A variant of a polynucleotide may be a naturally occurring variant such as a naturally occurring allelic variant, or it may be a variant that is not known to occur naturally. Such non-naturally occurring variants of the polynucleotide may be made by mutagenesis techniques, including those applied to polynucleotides, cells or organisms. Generally, differences are limited so that the nucleotide sequences of the reference and the variant are closely similar overall and, in many regions, identical.

Variants of polynucleotides according to the invention include, without being limited to, nucleotide sequences which are at least 95% identical to a polynucleotide selected from the group consisting. of the nucleotide sequences detailed in APPENDIX I or to any polynucleotide fragment of at least 8 consecutive nucleotides of a polynucleotide selected from the group detailed in APPENDIX I, and preferably at least 99% identical, more particularly at least 99.5% identical, and most preferably at least 99.8% identical to a polynucleotide selected from the group consisting of the nucleotide detailed in APPENDIX I or to any polynucleotide fragment of at least 30, 35, 40, 50, 70, 80, 100, 250, 500, 1000 or 2000, to the extent that the length is consistent with the particular sequence ID, consecutive nucleotides of a polynucleotide selected from the group consisting of the nucleotide sequences detailed in APPENDIX I.

Nucleotide changes present in a variant polynucleotide may be silent, which means that they do not alter the amino acids encoded by the polynucleotide. However, nucleotide changes may also result in amino acid substitutions, additions, deletions, fusions and truncations in the polypeptide encoded by the reference sequence. The substitutions, deletions or additions may involve one or more nucleotides. The variants may be altered in coding or non-coding regions or both. Alterations in the coding regions may produce conservative or non-conservative amino acid substitutions, deletions or additions.

A polynucleotide fragment is a polynucleotide having a sequence that is entirely the same as part but not all of a given nucleotide sequence, preferably the nucleotide sequence of an polynucleotide, and variants thereof, detailed in APPENDIX I. Such fragments may be “free-standing”, i.e. not part of or fused to other polynucleotides, or they may be comprised within a single larger polynucleotide of which they form a part or region. Indeed, several of these fragments may be present within a single larger polynucleotide. Optionally, such fragments may comprise, consist of, or consist essentially of a contiguous span of at least 8, 10, 12, 15, 18, 20, 25, 30, 35, 40, 50, 70, 80, 100, 250, 500, 1000 or 2000 nucleotides in length of any detailed in APPENDIX I.

Identity Between Nucleic Acids or Polypeptides

The terms “percentage of sequence identity” and “percentage homology” are used interchangeably herein to refer to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by, 100 to yield the percentage of sequence identity. Homology is evaluated using any of the variety of sequence comparison algorithms and programs known in the art. Such algorithms and programs include, but are by no means limited to, TBLASTN, BLASTP, FASTA, TFASTA, and CLUSTALW (Pearson and Lipman, 1988, Proc. Natl. Acad. Sci. USA 85(8):2444-2448; Altschul et al., 1990, J. Mol. Biol. 215(3):403-410; Thompson et al., 1994, Nucleic Acids Res. 22(2):4673-4680; Higgins et al. 1996, Methods Enzymol. 266:383-402; Altschul et al., 1990, J. Mol. Biol. 215(3):403-410; Altschul et al., 1993, Nature Genetics 3:266-272). In a particularly preferred embodiment, protein and nucleic acid sequence homologies are evaluated using the Basic Local Alignment Search Tool (“BLAST”) which is well known in the art (see, e.g., Karlin and Altschul, 1990, Proc. Natl. Acad. Sci. USA 87:2267-2268; Altschul et al., 1990, J. Mol. Biol. 215:403-410; Altschul et al., 1993, Nature Genetics 3:266-272; Altschul et al., 1997, Nuc. Acids Pes. 25:3389-3402). In particular, five specific BLAST programs are used to perform the following task:
(1) BLASTP and BLAST3 compare an amino acid query sequence against a protein sequence database;
(2) BLASTN compares a nucleotide query sequence against a nucleotide sequence database;
(3) BLASTX compares the six-frame conceptual translation products of a query nucleotide sequence (both strands) against a protein sequence database;
(4) TBLASTN compares a query protein sequence against a nucleotide sequence database translated in all six reading frames (both strands); and
(5) TBLASTX compares the six-frame translations of a nucleotide query sequence against the six-frame translations of a nucleotide sequence database.

The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (Gonnet et al., 1992, Science 256:1443-1445; Henikoff and Henikoff, 1993, Proteins 17:49-61). The BLAST programs evaluate the statistical significance of all high-scoring segment pairs identified, and preferably selects those segments which satisfy a user-specified threshold of significance, such as a user-specified percent homology. Preferably, the statistical significance of a high-scoring segment pair is evaluated using the statistical significance formula of Karlin (see, e.g., Karlin and Altschul, 1990, Proc. Natl. Acad. Sci. USA 87:2267-2268).

The BLAST programs may be used with the default parameters or with modified parameters provided by the user.

Stringent Hybridization Conditions

By way of example and not limitation, procedures using conditions of high stringency are as follows: Prehybridization of filters containing DNA is carried out for 8 h to overnight at 65.degree. C. in buffer composed of 6.times. SSC, 50 mM Tris-HCl (pH 7.5), 1 mM EDTA, 0.02% PVP, 0.02% Ficoll, 0.02% BSA, and 500 .mu.g/ml denatured salmon sperm DNA. Filters are hybridized for 48 h at 65.degree. C., the preferred hybridization temperature, in prehybridization mixture containing 100 .mu.g/ml denatured salmon sperm DNA and 5-20.times.10.sup.6 of .sup.32P-labeled probe. Subsequently, filter washes can be done at 37.degree. C. for 1 h in a solution containing 2.times.SSC, 0.01% PVP, 0.01% Ficoll, and 0.01% BSA, followed by a wash in 0.1 .times.SSC at 50.degree. C. for 45 min. Following the wash steps, the hybridized probes are detectable by autoradiography. Other conditions of high stringency which may be used are well known in the art and as cited in Sambrook et al., 1989; and Ausubel et al., 1989, are incorporated herein in their entirety. These hybridization conditions are suitable for a nucleic acid molecule of about 20 nucleotides in length. There is no need to say that the hybridization conditions described above are to be adapted according to the length of the desired nucleic acid, following techniques well known to the one skilled in the art. The suitable hybridization conditions may for example be adapted according to the teachings disclosed in the book of Hames and Higgins (1985) or in Sambrook et al. (1989).

Genomic Sequences of the Polynucleotides of the Invention

The present invention concerns genomic DNA sequences of the ADRBK2, BNDF, GSK3B, GRK3, IMPA1, IMPA2, INPP1, MARCKS, NTRK2 and NR1I2 genes, as well as DNA sequences of the human chromosome 22q11.23 region.

Preferred nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, 1000 or 2000 nucleotides of the sequences corresponding to the nucleotides detailed in APPENDIX I, or the complements thereof. Further nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, 1000 or 2000 nucleotides, to the extent that the length of said span is consistent with the length of the sequences corresponding to the nucleotides detailed in APPENDIX I. Optionally, said span is at least 12, 15, 18, 20, 25, 30, 35, 40, 50, 60, 70, 80, 90, 100, 150, 200, 500, 1000 or 2000 nucleotides of sequences corresponding to the nucleotides detailed in APPENDIX I.

Additional preferred nucleic acids of the invention include isolated, purified, or recombinant polynucleotides comprising a contiguous span of at least 12, 15, 18, 20, 25, 30, 35, 40, 45, 50, 55, 60, 65, 70, 75, 80, 90, 100 or 200 nucleotides of sequences corresponding to the nucleotides detailed in APPENDIX I or the complements thereof, wherein said contiguous span comprises a biallelic marker. It should be noted that nucleic acid fragments of any size and sequence may be comprised by the polynucleotides described in this section.

Another particularly preferred set of nucleic acids of the invention include isolated, purified, or recombinant polynucleotides comprising a contiguous span of at least 12, 15, 18, 20, 25, 30, 35, 40, 50, 60, 70, 80, 90, 100, 150, 200, 500, 1000 or 2000 nucleotides, to the extent that such a length is consistent with the lengths of the particular nucleotide position, of sequences corresponding to the nucleotides detailed in APPENDIX I or the complements thereof, wherein said contiguous span comprises at least 1, 2, 3, 5, or 10 nucleotide positions of any ranges of nucleotide positions.

The invention also encompasses a purified, isolated, or recombinant polynucleotide comprising a nucleotide sequence having at least 70, 75, 80, 85, 90, or 95% nucleotide identity with a nucleotide sequence of sequences corresponding to the nucleotides detailed in APPENDIX I or a complementary sequence thereto or a fragment thereof. The nucleotide differences as regards to nucleotide positions may be generally randomly distributed throughout the entire nucleic acid. Nevertheless, preferred nucleic acids are those wherein the nucleotide differences as regards to the sequences corresponding to the nucleotides detailed in APPENDIX I are predominantly located outside the coding sequences contained in the exons. These nucleic acids, as well as their fragments and variants, may be used as oligonucleotide primers or probes in order to detect the presence of a copy of the ADRBK2, BNDF, GSK3B, GRK3, IMPA1, IMPA2, INPP1, MARCKS, NTRK2 and NR1I2 gene(s) in a test sample, or alternatively in order to amplify a target nucleotide sequence within the 22q11.23 sequences.

Polynucleotides derived from 5′ and 3′ regulatory regions are useful in order to detect the presence of at least a copy of a nucleotide sequence detailed in APPENDIX I or a fragment thereof in a test sample. Polynucleotides carrying the regulatory elements located at the 5′ end and at the 3′ end of the genes comprising the exons of the present invention may be advantageously used to control the transcriptional and translational activity of a heterologous polynucleotide of interest.

In order to identify the relevant biologically active polynucleotide fragments or variants of an ADRBK2, BNDF, GSK3B, GRK3, IMPA1, IMPA2, INPP1, MARCKS, NTRK2 and/or NR1I2 regulatory region, one of skill in the art will refer to Sambrook et al. (1989), incorporated herein by reference, which describes the use of a recombinant vector carrying a marker gene (i.e. beta galactosidase, chloramphenicol acetyl transferase, etc.) the expression of which will be detected when placed under the control of a biologically active polynucleotide fragment or variant of the sequences corresponding to the nucleotides detailed in APPENDIX I. Genomic sequences located upstream of the first exon of the ADRBK2, BNDF, GSK3B, GRK3, IMPA1, IMPA2, INPP1, MARCKS, NTRK2 and/or NR1I2 gene(s) are cloned into a suitable promoter reporter vector, such as the pSEAP-Basic, pSEAP-Enhancer, p.beta.gal-Basic, p.beta.gal-Enhancer, or pEGFP-1 Promoter Reporter vectors available from Clontech, or pGL2-basic or pGL3-basic promoterless luciferase reporter gene vector from Promega. Briefly, each of these promoter reporter vectors include multiple cloning sites positioned upstream of a reporter gene encoding a readily assayable protein such as secreted alkaline phosphatase, luciferase, .beta. galactosidase, or green fluorescent protein. The sequences upstream of the ADRBK2, BNDF, GSK3B, GRK3, IMPA1, IMPA2, INPP1, MARCKS, NTRK2 and/or NR1I2 gene(s) coding region are inserted into the cloning sites upstream of the reporter gene in both orientations and introduced into an appropriate host cell. The level of reporter protein is assayed and compared to the level obtained from a vector which lacks an insert in the cloning site. The presence of an elevated expression level in the vector containing the insert with respect to the control vector indicates the presence of a promoter in the insert. If necessary, the upstream sequences can be cloned into vectors which contain an enhancer for increasing transcription levels from weak promoter sequences. A significant level of expression above that observed with the vector lacking an insert indicates that a promoter sequence is present in the inserted upstream sequence.

Promoter sequence within the upstream genomic DNA may be further defined by constructing nested 5′ and/or 3′ deletions in the upstream DNA using conventional techniques such as Exonuclease III or appropriate restriction endonuclease digestion. The resulting deletion fragments can be inserted into the promoter reporter vector to determine whether the deletion has reduced or obliterated promoter activity, such as described, for example, by Coles et al. (1998), the disclosure of which is incorporated herein by reference in its entirety. In this way, the boundaries of the promoters may be defined. If desired, potential individual regulatory sites within the promoter may be identified using site directed mutagenesis or linker scanning to obliterate potential transcription factor binding sites within the promoter individually or in combination. The effects of these mutations on transcription levels may be determined by inserting the mutations into cloning sites in promoter reporter vectors. This type of assay is well-known to those skilled in the art and is described in WO 97/17359, U.S. Pat. No. 5,374,544; EP 582 796; U.S. Pat. Nos. 5,698,389; 5,643,746; 5,502,176; and 5,266,488; the disclosures of which are incorporated by reference herein in their entirety.

The strength and the specificity of the promoter of the ADRBK2, BNDF, GSK3B, GRK3, IMPA1, IMPA2, INPP1, MARCKS, NTRK2 and/or NR1I2 gene(s) can be assessed through the expression levels of a detectable polynucleotide operably linked to the ADRBK2, BNDF, GSK3B, GRK3, IMPA1, IMPA2, INPP1, MARCKS, NTRK2 and NR1I2 gene(s) promoter in different types of cells and tissues. The detectable polynucleotide may be either a polynucleotide that specifically hybridizes with a predefined oligonucleotide probe, or a polynucleotide encoding a detectable protein, including an ADRBK2, BNDF, GSK3B, GRK3, IMPA1, IMPA2, INPP1, MARCKS, NTRK2 and NR1I2 gene(s) polypeptide or a fragment or a variant thereof. This type of assay is well-known to those skilled in the art and is described in U.S. Pat. Nos. 5,502,176; and 5,266,488; the disclosures of which are incorporated by reference herein in their entirety. Some of the methods are discussed in more detail below.

Polynucleotides carrying the regulatory elements located at the 5′ end and at the 3′ end of the ADRBK2, BNDF, GSK3B, GRK3, IMPA1, IMPA2, INPP1, MARCKS, NTRK2 and NR1I2 gene(s) coding region may be advantageously used to control the transcriptional and translational activity of an heterologous polynucleotide of interest.

The invention also pertains to a purified or isolated nucleic acid comprising a polynucleotide having at least 95% nucleotide identity with a polynucleotide selected from the group consisting of the 5′ and 3′ regulatory regions, advantageously 99% nucleotide identity, preferably 99.5% nucleotide identity and most preferably 99.8% nucleotide identity with a polynucleotide selected from the group consisting of the 5′ and 3′ regulatory regions, or a sequence complementary thereto or a variant thereof or a biologically active fragment thereof.

Another object of the invention consists of purified, isolated or recombinant nucleic acids comprising a polynucleotide that hybridizes, under the stringent hybridization conditions defined herein, with a polynucleotide selected from the group consisting of the nucleotide sequences of the 5′- and 3′ regulatory regions of ADRBK2, BNDF, GSK3B, GRK3, IMPA1, IMPA2, INPP1, MARCKS, NTRK2 and NR1I2 gene(s), or a sequence complementary thereto or a variant thereof or a biologically active fragment thereof.

Preferred fragments of the 5′ regulatory region have a length of about 1500 or 1000 nucleotides, preferably of about 500 nucleotides, more preferably about 400 nucleotides, even more preferably 300 nucleotides and most preferably about 200 nucleotides, while preferred fragments of the 3′ regulatory region are at least 50, 100, 150, 200, 300 or 400 bases in length.

“Biologically active” ADRBK2, BNDF, GSK3B, GRK3, IMPA1, IMPA2, INPP1, MARCKS, NTRK2 and/or NR1I2 gene(s) polynucleotide derivatives of nucleotides detailed in APPENDIX 1 are polynucleotides comprising or alternatively consisting in a fragment of said polynucleotide which is functional as a regulatory region for expressing a recombinant polypeptide or a recombinant polynucleotide in a recombinant cell host. It could act either as an enhancer or as a repressor.

For the purpose of the invention, a nucleic acid or polynucleotide is “functional” as a regulatory region for expressing a recombinant polypeptide or a recombinant polynucleotide if said regulatory polynucleotide contains nucleotide sequences which contain transcriptional and translational regulatory information, and such sequences are “operably linked” to nucleotide sequences which encode the desired polypeptide or the desired polynucleotide.

The regulatory polynucleotides of the invention may be prepared from the sequences corresponding to the nucleotides described in APPENDIX 1 by cleavage using suitable restriction enzymes, as described for example in Sambrook et al. (1989). The regulatory polynucleotides may also be prepared by digestion by an exonuclease enzyme, such as Bal31 (Wabiko et al., 1986). These regulatory polynucleotides can also be prepared by nucleic acid chemical synthesis, as described elsewhere in the specification.

The ADRBK2, BNDF, GSK3B, GRK3, IMPA1, IMPA2, INPP1, MARCKS, NTRK2 and NR1I2 gene(s) regulatory polynucleotides according to the invention may be part of a recombinant expression vector that may be used to express a coding sequence in a desired host cell or host organism. The recombinant expression vectors according to the invention are described elsewhere in the specification.

A preferred 5′-regulatory polynucleotide of the invention includes the 5′-untranslated region (5′-UTR) of the ADRBK2, BNDF, GSK3B, GRK3, IMPA1, IMPA2, INPP1, MARCKS, NTRK2 and/or NR1I2 gene(s) cDNA, or a biologically active fragment or variant thereof.

A preferred 3′-regulatory polynucleotide of the invention includes the 3′-untranslated region (3′-UTR) of the ADRBK2, BNDF, GSK3B, GRK3, IMPA1, IMPA2, INPP1, MARCKS, NTRK2 and/or NR1I2 gene(s) cDNA, or a biologically active fragment or variant thereof.

A further object of the invention consists of a purified or isolated nucleic acid comprising:
a) a nucleic acid comprising a regulatory nucleotide sequence selected from the group consisting of: (i) a nucleotide sequence comprising a polynucleotide of the ADRBK2, BNDF, GSK3B, GRK3, IMPA1, IMPA2, INPP1, MARCKS, NTRK2 and/or NR1I2 gene(s) 5′ regulatory region or a complementary sequence thereto; (ii) a nucleotide sequence comprising a polynucleotide having at least 95% of nucleotide identity with the nucleotide sequence of the ADRBK2, BNDF, GSK3B, GRK3, IMPA1, IMPA2, INPP1, MARCKS, NTRK2 and/or NR1I2 gene(s) 5′ regulatory region or a complementary sequence thereto; (iii) a nucleotide sequence comprising a polynucleotide that hybridizes under stringent hybridization conditions with the nucleotide sequence of the ADRBK2, BNDF, GSK3B, GRK3, IMPA1, IMPA2, INPP1, MARCKS, NTRK2 and/or NR1I2 gene(s) 5′ regulatory region or a complementary sequence thereto; and (iv) a biologically active fragment or variant of the polynucleotides in (i), (ii) and (iii); b) a polynucleotide encoding a desired polypeptide or a nucleic acid of interest, operably linked to the nucleic acid defined in (a) above; and c) optionally, a nucleic acid comprising a 3′-regulatory polynucleotide, preferably a 3′-regulatory polynucleotide of the ADRBK2, BNDF, GSK3B, GRK3, IMPA1, IMPA2, INPP1, MARCKS, NTRK2 and/or NR1I2 gene(s).

In a specific embodiment of the nucleic acid defined above, said nucleic acid includes the 5′-untranslated region (5′-UTR) of the ADRBK2, BNDF, GSK3B, GRK3, IMPA1, IMPA2, INPP1, MARCKS, NTRK2 and/or NR1I2 gene(s) cDNA, or a biologically active fragment or variant thereof.

In a second specific embodiment of the nucleic acid defined above, said nucleic acid includes the 3′-untranslated region (3′-UTR) of the ADRBK2, BNDF, GSK3B, GRK3, IMPA1, IMPA2, INPP1, MARCKS, NTRK2 and/or NR1I2 gene(s) cDNA, or a biologically active fragment or variant thereof.

The regulatory polynucleotide of the 5′ regulatory region, or its biologically active fragments or variants, is operably linked at the 5′-end of the polynucleotide encoding the desired polypeptide or polynucleotide.

The regulatory polynucleotide of the 3′ regulatory region, or its biologically active fragments or variants, is advantageously operably linked at the 3′-end of the polynucleotide encoding the desired polypeptide or polynucleotide.

The desired polypeptide encoded by the above-described nucleic acid may be of various nature or origin, encompassing proteins of prokaryotic or eukaryotic origin. Among the polypeptides expressed under the control of an ADRBK2, BNDF, GSK3B, GRK3, IMPA1, IMPA2, INPP1, MARCKS, NTRK2 and/or NR1I2 gene(s) regulatory region include bacterial, fungal or viral antigens. Also encompassed are eukaryotic proteins such as intracellular proteins, like “house keeping” proteins, membrane-bound proteins, like receptors, and secreted proteins like endogenous mediators such as cytokines. The desired polypeptide may be the ADRBK2, BNDF, GSK3B, GRK3, IMPA1, IMPA2, INPP1, MARCKS, NTRK2 and/or NR1I2 gene(s) protein, or a fragment or a variant thereof.

The desired nucleic acids encoded by the above-described polynucleotide, usually an RNA molecule, may be complementary to a desired coding polynucleotide, for example to the ADRBK2, BNDF, GSK3B, GRK3, IMPA1, IMPA2, INPP1, MARCKS, NTRK2 and/or NR1I2 gene(s) coding sequence, and thus useful as an antisense polynucleotide. Such a polynucleotide may be included in a recombinant expression vector in order to express the desired polypeptide or the desired nucleic acid in host cell or in a host organism. Suitable recombinant vectors that contain a polynucleotide such as described herein are disclosed elsewhere in the specification.

Oligonucleotide Probes and Primers

A probe or a primer according to the invention may be between 8 and 2000 nucleotides in length, or is specified to be at least 12, 15, 18, 20, 25, 35, 40, 50, 60, 70, 80, 100, 250, 500, 1000 nucleotides in length. More particularly, the length of these probes can range from 8, 10, 15, 20, or 30 to 100 nucleotides, preferably from 10 to 50, more preferably from 15 to 30 nucleotides. Shorter probes tend to lack specificity for a target nucleic acid sequence and generally require cooler temperatures to form sufficiently stable hybrid complexes with the template. Longer probes are expensive to produce and can sometimes self-hybridize to form hairpin structures. The appropriate length for primers and probes under a particular set of assay conditions may be empirically determined by one of skill in the art.

The primers and probes can be prepared by any suitable method, including, for example, cloning and restriction of appropriate sequences and direct chemical synthesis by a method such as the phosphodiester method of Narang et al. (1979), the phosphodiester method of Brown et al. (1979), the diethylphosphoramidite method of Beaucage et al. (1981) and the solid support method described in EP 0 707 592. The disclosures of all these documents are incorporated herein by reference.

Detection probes are generally nucleic acid sequences or uncharged nucleic acid analogs such as, for example peptide nucleic acids which are disclosed in International Patent Application WO 92/20702, morpholino analogs which are described in U.S. Pat. Nos. 5,185,444; 5,034,506 and 5,142,047, the disclosures of which are all incorporated herein by reference. The probe may have to be rendered “non-extendable” in that additional dNTPs cannot be added to the probe. In and of themselves analogs usually are non-extendable and nucleic acid probes can be rendered non-extendable by modifying the 3′ end of the probe such that the hydroxyl group is no longer capable of participating in elongation. For example, the 3′ end of the probe can be functionalized with the capture or detection label to thereby consume or otherwise block the hydroxyl group. Alternatively, the 3′ hydroxyl group simply can be cleaved, replaced or modified; U.S. patent application Ser. No. 07/049,061 filed Apr. 19, 1993, incorporated herein by reference, describes modifications which can be used to render a probe non-extendable.

Any of the polynucleotides of the present invention can be labeled, if desired, by incorporating a label detectable by spectroscopic, photochemical, biochemical, immunochemical, or chemical means. For example, useful labels include radioactive substances (.sup.32P, .sup.35S, .sup.3H, .sup.1251), fluorescent dyes (5-bromodesoxyuridin, fluorescein, acetylaminofluorene, digoxigenin) or biotin. Preferably, polynucleotides are labeled at their 3′ and 5′ ends. Examples of non-radioactive labeling of nucleic acid fragments are described in the French patent No. FR-7810975 or by Urdea et al (1988) or Sanchez-Pescador et al (1988), each incorporated herein by reference. In addition, the probes according to the present invention may have structural characteristics such that they allow the signal amplification, such structural characteristics being, for example, branched DNA probes as those described by Urdea et al. in 1991 or in the European patent No. EP 0 225 807 (Chiron), incorporated herein by reference.

A label can also be used to capture the primer, so as to facilitate the immobilization of either the primer or a primer extension product, such as amplified DNA, on a solid support. A capture label is attached to the primers or probes and can be a specific binding member which forms a binding pair with the solid phase reagent's specific binding member (e.g. biotin and streptavidin). Therefore depending upon the type of label carried by a polynucleotide or a probe, it may be employed to capture or to detect the target DNA. Further, it will be understood that the polynucleotides, primers or probes provided herein, may, themselves, serve as the capture label. For example, in the case where a solid phase reagent's binding member is a nucleic acid sequence, it may be selected such that it binds a complementary portion of a primer or probe to thereby immobilize the primer or probe to the solid phase. In cases where a polynucleotide probe itself serves as the binding member, those skilled in the art will recognize that the probe will contain a sequence or “tail” that is not complementary to the target. In the case where a polynucleotide primer itself serves as the capture label, at least a portion of the primer will be free to hybridize with a nucleic acid on a solid phase. DNA Labeling techniques are well known to the skilled technician.

The probes of the present invention are useful for a number of purposes. They can be notably used in Southern hybridization to genomic DNA. The probes can also be used to detect PCR amplification products. They may also be used to detect mismatches in a sequence comprising a polynucleotide of the ADRBK2, BNDF, GSK3B, GRK3, IMPA1, IMPA2, INPP1, MARCKS, NTRK2 and/or NR1I2 gene(s) or mRNA using other techniques.

Any of the polynucleotides, primers and probes of the present invention can be conveniently immobilized on a solid support. Solid supports are known to those skilled in the art and include the walls of wells of a reaction tray, test tubes, polystyrene beads, magnetic beads, nitrocellulose strips, membranes, microparticles such as latex particles, sheep (or other animal) red blood cells, duracytes and others. The solid support is not critical and can be selected by one skilled in the art. Thus, latex particles, microparticles, magnetic or non-magnetic beads, membranes, plastic tubes, walls of microtiter wells, glass or silicon chips, sheep (or other suitable animal's) red blood cells and duracytes are all suitable examples. Suitable methods for immobilizing nucleic acids on solid phases include ionic, hydrophobic, covalent interactions and the like. A solid support, as used herein, refers to any material which is insoluble, or can be made insoluble by a subsequent reaction. The solid support can be chosen for its intrinsic ability to attract and immobilize the capture reagent. Alternatively, the solid phase can retain an additional receptor which has the ability to attract and immobilize the capture reagent. The additional receptor can include a charged substance that is oppositely charged with respect to the capture reagent itself or to a charged substance conjugated to the capture reagent. As yet another alternative, the receptor molecule can be any specific binding member which is immobilized upon (attached to) the solid support and which has the ability to immobilize the capture reagent through a specific binding reaction. The receptor molecule enables the indirect binding of the capture reagent to a solid support material before the performance of the assay or during the performance of the assay. The solid phase thus can be a plastic, derivatized plastic, magnetic or non-magnetic metal, glass or silicon surface of a test tube, microtiter well, sheet, bead, microparticle, chip, sheep (or other suitable animal's) red blood cells, duracytes and other configurations known to those of ordinary skill in the art. The polynucleotides of the invention can be attached to or immobilized on a solid support individually or in groups of at least 2, 5, 8, 10, 12, 15, 20, or 25 distinct polynucleotides of the invention to a single solid support. In addition, polynucleotides other than those of the invention may be attached to the same solid support as one or more polynucleotides of the invention.

Consequently, the invention also comprises a method for detecting the presence of a nucleic acid comprising a nucleotide sequence selected from a group detailed in APPENDIX 1, a fragment or a variant thereof or a complementary sequence thereto in a sample, said method comprising the following steps of: a) bringing into contact a nucleic acid probe or a plurality of nucleic acid probes which can hybridize with a nucleotide sequence included in a nucleic acid selected from the group consisting of the sequences corresponding to the nucleotides detailed in APPENDIX 1, a fragment or a variant thereof or a complementary sequence thereto and the sample to be assayed; and b) detecting the hybrid complex formed between the probe and a nucleic acid in the sample.

The invention further concerns a kit for detecting the presence of a nucleic acid comprising a nucleotide sequence selected from a group sequences corresponding to the nucleotides detailed in APPENDIX I, a fragment or a variant thereof or marker in linkage disequilibrium thereto in a sample, said kit comprising: a) a nucleic acid probe or a plurality of nucleic acid probes which can hybridize with a nucleotide sequence included in a nucleic acid selected form the group consisting of the nucleotide sequences corresponding to the nucleotides detailed in APPENDIX I, a fragment or a variant thereof or a complementary sequence thereto; and b) optionally, the reagents necessary for performing the hybridization reaction.

In a first preferred embodiment of this detection method and kit, said nucleic acid probe or the plurality of nucleic acid probes are labeled with a detectable molecule. In a second preferred embodiment of said method and kit, said nucleic acid probe or the plurality of nucleic acid probes has been immobilized on a substrate. In a third preferred embodiment, the nucleic acid probe or the plurality of nucleic acid probes comprise either a sequence which is selected from the group consisting of the nucleotide sequences of the SNPs primers detailed in APPENDIX I, and the complementary sequence thereto.

Oligonucleotide Arrays

A substrate comprising a plurality of oligonucleotide primers or probes of the invention may be used either for detecting or amplifying targeted sequences in a nucleotide sequence of the SNPs detailed in APPENDIX I, more particularly in an ADRBK2, BNDF, GSK3B, GRK3, IMPA1, IMPA2, INPP1, MARCKS, NTRK2 and/or NR1I2 ADRBK2, BNDF, GSK3B, GRK3, IMPA1, IMPA2, INPP1, MARCKS, NTRK2 and/or NR1I2 polynucleotide, or in genes comprising an ADRBK2, BNDF, GSK3B, GRK3, IMPA1, IMPA2, INPP1, MARCKS, NTRK2 and/or NR1I2 polynucleotide and may also be used for detecting mutations in the coding or in the non-coding sequences of an ADRBK2, BNDF, GSK3B, GRK3, IMPA1, IMPA2, INPP1, MARCKS, NTRK2 and/or NR1I2 nucleic acid sequence, or genes comprising an ADRBK2, BNDF, GSK3B, GRK3, IMPA1, IMPA2, INPP1, MARCKS, NTRK2 and/or NR1I2nucleic acid sequence.

Any polynucleotide provided herein may be attached in overlapping areas or at random locations on the solid support. Alternatively the polynucleotides of the invention may be attached in an ordered array wherein each polynucleotide is attached to a distinct region of the solid support which does not overlap with the attachment site of any other polynucleotide. Preferably, such an ordered array of polynucleotides is designed to be “addressable” where the distinct locations are recorded and can be accessed as part of an assay procedure. Addressable polynucleotide arrays typically comprise a plurality of different oligonucleotide probes that are coupled to a surface of a substrate in different known locations. The knowledge of the precise location of each polynucleotides location makes these “addressable” arrays particularly useful in hybridization assays. Any addressable array technology known in the art can be employed with the polynucleotides of the invention. One particular embodiment of these polynucleotide arrays is known as Genechips.TM., and has been generally described in U.S. Pat. No. 5,143,854; PCT publications WO 90/15070 and 92/10092, the disclosures of which are incorporated herein by reference. These arrays may generally be produced using mechanical synthesis methods or light directed synthesis methods which incorporate a combination of photolithographic methods and solid phase oligonucleotide synthesis (Fodor et al., 1991, incorporated herein by reference). The immobilization of arrays of oligonucleotides on solid supports has been rendered possible by the development of a technology generally identified as “Very Large Scale Immobilized Polymer Synthesis” (VLSIPS.TM.) in which, typically, probes are immobilized in a high density array on a solid surface of a chip. Examples of VLSIPS.TM. technologies are provided in U.S. Pat. Nos. 5,143,854; and 5,412,087 and in PCT Publications WO 90/15070, WO 92/10092 and WO 95/11995, each of which are incorporated herein by reference, which describe methods for forming oligonucleotide arrays through techniques such as light-directed synthesis techniques. In designing strategies aimed at providing arrays of nucleotides immobilized on solid supports, further presentation strategies were developed to order and display the oligonucleotide arrays on the chips in an attempt to maximize hybridization patterns and sequence information. Examples of such presentation strategies are disclosed in PCT Publications WO 94/12305, WO 94/11530, WO 97/29212 and WO 97/31256, the disclosures of which are incorporated herein by reference in their entireties.

In another embodiment of the oligonucleotide arrays of the invention, an oligonucleotide probe matrix may advantageously be used to detect mutations occurring in an ADRBK2, BNDF, GSK3B, GRK3, IMPA1, IMPA2, INPP1, MARCKS, NTRK2 and/or NR1I2 gene(s). For this particular purpose, probes are specifically designed to have a nucleotide sequence allowing their hybridization to the genes that carry known mutations (either by deletion, insertion or substitution of one or several nucleotides). By known mutations in an ADRBK2, BNDF, GSK3B, GRK3, IMPA1, IMPA2, INPP1, MARCKS, NTRK2 and/or NR1I2 polynucleotide, it is meant, mutations in an ADRBK2, BNDF, GSK3B, GRK3, IMPA1, IMPA2, INPP1, MARCKS, NTRK2 and/or NR1I2 polynucleotide that have been identified according; the technique used by Huang et al. (1996) or Samson et al. (1996), each incorporated herein by reference, for example, may be used to identify such mutations.

Another technique that is used to detect mutations in an ADRBK2, BNDF, GSK3B, GRK3, IMPA1, IMPA2, INPP1, MARCKS, NTRK2 and/or NR1I2 polynucleotide is the use of a high-density DNA array. Each oligonucleotide probe constituting a unit element of the high density DNA array is designed to match a specific subsequence of an ADRBK2, BNDF, GSK3B, GRK3, IMPA1, IMPA2, INPP1, MARCKS, NTRK2 and/or NR1I2 polynucleotide. Thus, an array consisting of oligonucleotides complementary to subsequences of the target gene sequence is used to determine the identity of the target sequence with the wild-type gene sequence, measure its amount, and detect differences between the target sequence and the reference wild-type nucleic acid sequence of an ADRBK2, BNDF, GSK3B, GRK3, IMPA1, IMPA2, INPP1, MARCKS, NTRK2 and/or NR1I2 polynucleotide. In one such design, termed 4L tiled array, is implemented a set of four probes (A, C, G, T), preferably 15-nucleotide oligomers. In each set of four probes, the perfect complement will hybridize more strongly than mismatched probes. Consequently, a nucleic acid target of length L is scanned for mutations with a tiled array containing 4L probes, the whole probe set containing all the possible mutations in the known wild reference sequence. The hybridization signals of the 15-mer probe set tiled array are perturbed by a single base change in the target sequence. As a consequence, there is a characteristic loss of signal or a “footprint” for the probes flanking a mutation position. This technique was described by Chee et al. in 1996, which is herein incorporated by reference.

Consequently, the invention concerns an array of nucleic acid molecules comprising at least one polynucleotide described above as probes and primers. Preferably, the invention concerns an array of nucleic acid comprising at least two polynucleotides described above as probes and primers.

ADRBK2, BNDF, GSK3B, GRK3, IMPA1, IMPA2, INPP1, MARCKS, NTRK2 and/or NR1I2 Proteins and Polypeptide Fragments

The terms “ADRBK2, BNDF, GSK3B, GRK3, IMPA1, IMPA2, INPP1, MARCKS, NTRK2 and/or NR1I2 polypeptides” are used herein to embrace all of the proteins and polypeptides encoded by the respective ADRBK2, BNDF, GSK3B, GRK3, IMPA1, IMPA2, INPP1, MARCKS, NTRK2 and/or NR1I2 polypeptides of the present invention. Forming part of the invention are polypeptides encoded by the polynucleotides of the invention, as well as fusion polypeptides comprising such polypeptides. The invention embodies proteins from humans, mammals, primates, non-human primates, and includes isolated or purified ADRBK2, BNDF, GSK3B, GRK3, IMPA1, IMPA2, INPP1, MARCKS, NTRK2 and/or NR1I2 proteins.

It should be noted that the ADRBK2, BNDF, GSK3B, GRK3, IMPA1, IMPA2, INPP1, MARCKS, NTRK2 and/or NR1I2 proteins of the invention also comprise naturally-occurring variants of the amino acid sequence of the respective human ADRBK2, BNDF, GSK3B, GRK3, IMPA1, IMPA2, INPP1, MARCKS, NTRK2 and/or NR1I2 proteins.

The present invention embodies isolated, purified, and recombinant polypeptides comprising a contiguous span of at least 4 amino acids, preferably at least 6, more preferably at least 8 to 10 amino acids, more preferably at least 12, 15, 20, 25, 30, 40, 50, or 100 amino acids, to the extent that said span is consistent with the length of a particular polypeptide of such sequences corresponding to the nucleotides detailed in APPENDIX I. In other preferred embodiments the contiguous stretch of amino acids comprises the site of a mutation or functional mutation, including a deletion, addition, swap or truncation of the amino acids in an ADRBK2, BNDF, GSK3B, GRK3, IMPA1, IMPA2, INPP1, MARCKS, NTRK2 and/or NR1I2 protein sequence.

The invention also embodies isolated, purified, and recombinant ADRBK2, BNDF, GSK3B, GRK3, IMPA1, IMPA2, INPP1, MARCKS, NTRK2 and/or NR1I2 polypeptides comprising a contiguous span of at least 4 amino acids, preferably at least 6 or at least 8 to 10 amino acids, more preferably at least 12, 15, 20, 25, 30, 40, 50, or 100 amino acids.

Biological samples may comprise any tissue or cell preparation in which at least one nucleic acid sequence corresponding to a human DNA sequence, and in preferred embodiments corresponding to a gene sequence, can be detected, and may vary in nature accordingly, depending on the particular sequence(s) to be compared. Biological samples may be provided by obtaining a blood sample, biopsy specimen, tissue explant, organ culture or any other tissue or cell preparation from a subject or a biological source. The subject or biological source may be a human or non-human animal, a primary cell culture or culture adapted cell line including but not limited to genetically engineered cell lines that may contain chromosomally integrated or episomal recombinant nucleic acid sequences, immortalized or immortalizable cell lines, somatic cell hybrid or cytoplasmic hybrid “cybrid” cell lines, differentiated or differentiatable cell lines, transformed cell lines and the like. In certain preferred embodiments of the invention, the biological sample may be derived from a subject or biological source suspected of having or being at risk for having bipolar disorder, and in certain preferred embodiments of the invention the subject or biological source may be known to be free of a risk or presence of such as disease.

The term “tissue” includes blood and/or cells isolated or suspended from solid body mass, as well as the solid body mass of the various organs. “Immortal” cell lines denotes cell lines that are so denoted by persons of ordinary skill, or are capable of being passaged preferably an indefinite number of times, but not less than ten times, without significant phenotypic alteration. “.rho..sup.0 cells” are cells essentially depleted of functional mitochondria and/or mitochondrial DNA, by any method useful for this purpose.

The term “mood disorder” is used in the claims to denote the disease that exhibits the symptoms of mood disorder recognizable to one of ordinary skill in the art. “Mood Disorders” or may include, but need not be limited to, depression, bipolar disorder, schizophrenia, mania, attention-deficit disorder, and anxiety. For example, where it is desirable to determine whether or not a subject falls within clinical parameters indicative of bipolar disorder, signs and symptoms of bipolar disorder that are accepted by those skilled in the art may be used to so designate a subject, such as, e.g., clinical signs defined in the DSM-IV. as 4 or more depressions, hypomanias, or manias in the prior 12 month period, with episodes being demarcated by a switch to an episode of opposite polarity or by a period of remission, or other means known in the art for diagnosing mood disorders. A phenotypic trait, symptom, mutation or condition “correlates” with bipolar disorder if it is repeatedly observed in individuals diagnosed as having some form of bipolar disorder, or if it is routinely used by persons of ordinary skill in the art as a diagnostic criterion in determining that an individual has bipolar disorder or a related condition.

The DSM-IV classification of bipolar disorder distinguishes among four types of disorders based on the degree and duration of mania or hypomania as well as two types of disorders which are evident typically with medical conditions or their treatments, or to substance abuse. Mania is recognized by elevated, expansive or irritable mood as well as by distractability, impulsive behavior, increased activity, grandiosity, elation, racing thoughts, and pressured speech. Of the four types of bipolar disorder characterized by the particular degree and duration of mania, DSM-IV includes:
bipolar disorder I, including patients displaying mania for at least one week;
bipolar disorder II, including patients displaying hypomania for at least 4 days, characterized by milder symptoms of excitement than mania, who have not previously displayed mania, and have previously suffered from episodes of major depression;
bipolar disorder not otherwise specified (NOS), including patients otherwise displaying features of bipolar disorder II but not meeting the 4 day duration for the excitement phase, or who display hypomania without an episode of major depression; and
cyclothymia, including patients who show numerous manic and depressive symptoms that do not meet the criteria for hypomania or major depression, but which are displayed for over two years without a symptom-free interval of more than two months.

The remaining two types of bipolar disorder as classified in DSM-IV are disorders evident or caused by various medical disorder and their treatments, and disorders involving or related to substance abuse. Medical disorders which can cause bipolar disorders typically include endocrine disorders and cerebrovascular injuries, and medical treatments causing bipolar disorder are known to include glucocorticoids and the abuse of stimulants. The disorder associated with the use or abuse of a substance is referred to as “substance induced mood disorder with manic or mixed features”.

Diagnosis of bipolar disorder can be very challenging. One particularly troublesome difficulty is that some patients exhibit mixed states, simultaneously manic and dysphoric or depressive, but do not fall into the DSM-IV classification because not all required criteria for mania and major depression are met daily for at least one week. Other difficulties include classification of patients in the DSM-IV groups based on duration of phase since patients often cycle between excited and depressive episodes at different rates. In particular, it is reported that the use of antidepressants may alter the course of the disease for the worse by causing “rapid-cycling”. Also making diagnosis more difficult is the fact that bipolar patients, particularly at what is known as Stage III mania, share symptoms of disorganized thinking and behavior with bipolar disorder patients. Furthermore, psychiatrists must distinguish between agitated depression and mixed mania; it is common that patients with major depression (14 days or more) exhibit agitation, resulting in bipolar-like features. A yet further complicating factor is that bipolar patients have an exceptionally high rate of substance, particularly alcohol abuse. While the prevalence of mania in alcoholic patients is low, it is well known that substance abusers can show excited symptoms. Difficulties therefore result for the diagnosis of bipolar patients with substance abuse.

Pre-clinical and/or asymptomatic conditions that correlate with the presence of DNA mutations often observed in patients with bipolar disorder, such as GSK3beta, may represent steps in the progression in the disease. Individuals that lack the full panoply of such symptoms but carry mutations that “correlate” with bipolar disorder are hereby defined as being “at risk” or having a “predisposition” for developing the fully symptomatic disease.

Although the invention focuses preferentially on humans afflicted with or at risk for developing bipolar disorder as defined above and the treatment thereof, the invention also encompasses the analysis of tissues and preparation, from relatives of persons having or being “at risk” for developing bipolar disorder (which relatives may or may not themselves be at risk), and, in vivo and in vitro animal and tissue culture models that may exhibit one or more or all of the symptoms that correlate with the genomic mutations of the invention.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

Although the cells suggested for certain embodiments herein are immortalized neuronal tissue and cells, myoblasts and insulin-responsive cells and platelets, the present invention is not limited to the use of such cells. Cells from different tissues (breast epithelium, colon, lymphocytes, etc.) or different species (human, mouse, etc.) are also useful in the present invention.

Throughout this application various publications are referenced within parentheses. The disclosures of these publications in their entireties are hereby incorporated by reference in this application.

Pharmacogenomics of Lithium in Treatment of Bipolar Disorder

Although lithium has been demonstrated to be effective in the treatment of bipolar disorder in a certain percentage of patients, its mechanism of action is unclear. Its primary site of action has been proposed to be signal transduction mechanisms, however, its effects on neurotransmitter systems has also been reported. A number of signaling proteins have been proposed as potential targets of lithium action, including inositol monophosphatase (IMPase), inositol polyphosphatase (IPPase) and the protein kinase, glycogen synthase kinase-3 (GSK-3beta). These potential targets are widely expressed, require metal ions for catalysis, and are generally inhibited by lithium (see for instance Phiel C J, Klein P S. (2001) Molecular targets of lithium action. Annu Rev Pharmacol Toxicol 41(789-813.); Lenox R H, Hahn C G. (2000) Overview of the mechanism of action of lithium in the brain: fifty-year update. J Clin Psychiatry 61 Suppl 9(5-15.)).

Lithium has also been demonstrated to affect the anti-apoptotic protein, Bcl-2, which may indicate the drugs involvement in neuroprotective effects (Manji H K, Moore G J, Chen G. (2000) Lithium up-regulates the cytoprotective protein Bcl-2 in the CNS in vivo: a role for neurotrophic and neuroprotective effects in manic depressive illness. J Clin Psychiatry 61 Suppl 9(82-96.)), although it is likely that this effect is secondary to the drug's effect has on the signal transduction pathway (involving IP3) upstream of this molecule.

These systems have been critically analyzed in the following sections to identify the most relevant candidates for gene/SNP sets to be used as system inputs for our predictive algorithm. SNPs for selected genes are listed in Appendix I.

Metabolism of Lithium

Lithium is a cation and is not metabolized. It is cleared through the kidneys, intact.

Glutamate Transporters

Glutamate is an excitatory neurotransmitter and its reduction has been proposed to exert an antimanic effect. Chronic lithium administration upregulates glutamate reuptake thereby decreasing glutamate availability in synapse. (Shaldubina A, Agam G, Belmaker RH. (2001) The mechanism of lithium action: state of the art, ten years later. Prog Neuropsychopharmacol Biol Psychiatry 25(4): 855-66.). This is performed by several (Na2+/K+)-coupled glutamate transporters.

Glutamate transporters function in a two-stage electrogenic process, in which glutamate is first cotransported with three sodium ions and a proton. Subsequently, the cycle is completed by translocation of a potassium ion in the opposite direction. While the wild-type GLT-1 (human homologue, EAAT2) transporter is strictly dependent on sodium, it has been shown that the selectivity of Y403F mutant transporter is altered so that sodium can be replaced by other alkaline metal cations including lithium and cesium (Zhang Y, Bendahan A, Zarbiv R, Kavanaugh M P, Kanner B I. (1998) Molecular determinant of ion selectivity of a (Na++K+)-coupled rat brain glutamate transporter. Proc Natl Acad Sci U S A 95(2): 751-5.). The ramifications of this have not yet been elucidated, but this represents potential for system affectation by lithium exposure in such mutants. Additionally, it has been demonstrated that in the wild-type EAAC1 transporter (human homologue, EAAT3) lithium can replace sodium in coupled uptake. However, in two mutants, T370S and G410S, lithium is unable to support coupled transport (Borre L, Kanner B I. (2001) Coupled, but not uncoupled, fluxes in a neuronal glutamate transporter can be activated by lithium ions. J Biol Chem 276(44): 40396-401.). Again, ramifications have not yet been elucidated, but this represents potential for system affectation by lithium exposure in such mutants.

Glutamate Receptor

Results suggest that modulation of glutamate receptor hyperactivity represents at least part of the molecular mechanisms by which lithium alters brain function and exerts its clinical efficacy in the treatment for manic depressive illness (Nonaka S, Hough C J, Chuang D M. (1998) Chronic lithium treatment robustly protects neurons in the central nervous system against excitotoxicity by inhibiting N-methyl-D-aspartate receptor-mediated calcium influx. Proc Natl Acad Sci U S A 95(5): 2642-7.).

Long-term exposure to lithium chloride dramatically protects cultured rat cerebellar, cerebral cortical, and hippocampal neurons against glutamate-induced excitotoxicity, which involves apoptosis mediated by N-methyl-D-aspartate (NMDA) receptors. Although it has been shown that this effect of lithium is not caused by down-regulation of NMDA receptor subunit proteins and is not proposed to be related to lithium's ability to block inositol monophosphatase activity (Nonaka S, Hough C J, Chuang D M. (1998) Chronic lithium treatment robustly protects neurons in the central nervous system against excitotoxicity by inhibiting N-methyl-D-aspartate receptor-mediated calcium influx. Proc Natl Acad Sci U S A 95(5): 2642-7.), the mechanism of suppression is as of yet unknown.

While it has been demonstrated that modulation of NMDA receptor activity is likely to be involved in the cascade of events that result in response to lithium treatment, it does not appear that this receptor is directly affected by lithium. Instead, it appears that lithium's inhibition/activation of other factors may be responsible for this action. For this reason we do not consider the NMDA receptor subunits as primary components in development of our algorithm for prediction of response to lithium.

Serotonergic, Dopaminergic, and Noradrenergic Systems

The serotonergic, dopaminergic, noradrenergic systems have been associated with the pathogenesis of bipolar disorder (Lerer B, Macciardi F, Segman R H, Adolfsson R, Blackwood D, Blairy S, Del Favero J, Dikeos D G, Kaneva R, Lilli R, Massat I, Milanova V, Muir W, Noethen M, Oruc L, Petrova T, Papadimitriou G N, Rietschel M, Serretti A, Souery D, Van Gestel S, Van Broeckhoven C, Mendlewicz J. (2001) Variability of 5-HT2C receptor cys23ser polymorphism among European populations and vulnerability to affective disorder. Mol Psychiatry 6(5): 579-85.). Each receptor system has been reported in the literature to be affected by lithium treatment. Effects on their correlate transporters, however, are less clear.

In the Serotonin pathway, reports indicate that lithium targets and affects the 5-HT1A (See for instance Subhash M N, Vinod K Y, Srinivas B N. (1999) Differential effect of lithium on 5-HT1 receptor-linked system in regions of rat brain. Neurochem Int 35(4): 337-43.; Odagaki Y. (1992) [Effects of lithium and antidepressants on monoaminergic receptors and receptor-coupled adenylate cyclase system in rat brain]. Hokkaido Igaku Zasshi 67(2): 247-58.; Fujii T, Nakai K, Nakajima Y, Kawashima K. (2000) Enhancement of hippocampal cholinergic neurotransmission through 5-HT1A receptor-mediated pathways by repeated lithium treatment in rats. Can J Physiol Pharmacol 78(5): 392-9.; Muraki I. (2001) [Behavioral and neurochemical study on the mechanism of the anxiolytic effect of a selective serotonin reuptake inhibitor, a selective serotonin1A agonist and lithium carbonate]. Hokkaido Igaku Zasshi 76(2): 57-70.; and Wegener G, Smith D F, Rosenberg R. (1997) 5-HT1A receptors in lithium-induced conditioned taste aversion. Psychopharmacology (Ber1) 133(1): 51-4.), 5-HT1B (see for instance Redrobe J P, Bourin M. (1999) Evidence of the activity of lithium on 5-HT1B receptors in the mouse forced swimming test: comparison with carbamazepine and sodium valproate. Psychopharmacology (Ber1) 141(4): 370-7.; andMassot O, Rousselle J C, Fillion M P, Januel D, Plantefol M, Fillion G. (1999) 5-HT1B receptors: a novel target for lithium. Possible involvement in mood disorders. Neuropsychopharmacology 21(4): 530-41.), 5-HT2A(Moorman J M, Leslie R A. (1998) Paradoxical effects of lithium on serotonergic receptor function: an immunocytochemical, behavioural and autoradiographic study. Neuropharmacology 37(3): 357-74.) and 5-HT2C(see for instance Moorman J M, Leslie R A. (1998) Paradoxical effects of lithium on serotonergic receptor function: an immunocytochemical, behavioural and autoradiographic study. Neuropharmacology 37(3): 357-74.; and Matsuoka T, Nishizaki T, Sumino K. (1997) A specific inhibitory action of lithium on the 5-HT2c receptor expressed in Xenopus laevis oocytes. Mol Pharmacol 51(3): 471-4.) receptors.

A potential role of dopamine in bipolar disorder has been suggested namely due to the ability of dopaminergic agonists to induce mania (Mitchell P, Selbie L, Waters B, Donald J, Vivero C, Tully M, Shine J. (1992) Exclusion of close linkage of bipolar disorder to dopamine D1 and D2 receptor gene markers. J Affect Disord 25(1): 1-11.). Lithium has been shown to directly reduce the actions of D1 receptor agonists (Acquas E, Fibiger H C. (1996) Chronic lithium attenuates dopamine D1-receptor mediated increases in acetylcholine release in rat frontal cortex. Psychopharmacology (Ber1) 125(2): 162-7.), thereby indicating a direct effect of lithium on this receptor.

In the Noradrenaline pathway, Lithium has been reported to stimulate the desensitization and internalization of beta2-adrenergic receptors (Doronin S, Shumay E, Wang H, Malbon C C. (2001) Lithium Suppresses Signaling and Induces Rapid Sequestration of beta2-Adrenergic Receptors. Biochem Biophys Res Commun 288(1): 151-5.). It has not been shown to have a direct effect on this receptor. For this reason, this receptor has not been chosen for use in system development.

There is a report that replacement of Na+ with Li+ inhibits the noradrenaline, serotonin and dopamine transporters from between 94-100% in varying cell types (Bryan-Lluka L J, Bonisch H. (1997) Lanthanides inhibit the human noradrenaline, 5-hydroxytryptamine and dopamine transporters. Naunyn Schmiedebergs Arch Pharmacol 355(6): 699-706.). However, this finding has not been replicated, and others have reported that lithium does not inhibit serotonin uptake (see for instance Southam E, Kirkby D, Higgins G A, Hagan R M. (1998) Lamotrigine inhibits monoamine uptake in vitro and modulates 5-hydroxytryptamine uptake in rats. Eur J Pharmacol 358(1): 19-24.; and El Khoury A, Johnson L, berg-Wistedt A, Stain-Malmgren R. (2001) Effects of long-term lithium treatment on monoaminergic functions in major depression. Psychiatry Res 105(1-2): 33-44.) and may actually increase dopamine uptake (47) via the respective transporters.

Inositol mono- and polyphosphatase (IMPase, IPPase)

A primary hypothesis in the mechanism of action of lithium is “inositol depletion.” Patients with bipolar have been noted to have hyperactive signaling in the inositol pathway. Attenuation of this signaling to achieve symptom remission is achieved by “inositol depletion.” Myo-inositol is required for the formation of PIP2.

The paradigm of inositol signaling activation is that receptor stimulation activates phsopholipase C (PLC) leading to the breakdown of phosphatidylinositol-(4,5)-bisphosphate (PIP2) into the two second messengers, inositol 1,4,5 trisphosphate (IP3) and diacyl glycerol (DAG). IP3 regulates Ca2+via the IP3 receptor which then affects PKC. Diacyl glycerol also has effects on PKC.

Levels of PIP2 are maintained via recycling of IP3. Recycling occurs by breakdown of IP3 via inositol polyphosphatase (IPPase) and inositol monophosphotase (IMPase) which together produce myo-inositol, a basic building block of PIP2. Lithium inhibits IMPase and IPPase which limits the amount of myo-inositol available for recycling and thereby attenuates the signaling activity—presumably to the levels observed in healthy patients. (see for instance Fauroux C M, Freeman S. (1999) Inhibitors of inositol monophosphatase. J Enzyme Inhib 14(2): 97-108.; O'Donnell T, Rotzinger S, Nakashima T T, Hanstock C C, Ulrich M, Silverstone P H. (2000) Chronic lithium and sodium valproate both decrease the concentration of myo-inositol and increase the concentration of inositol monophosphates in rat brain. Brain Res 880(1-2): 84-91.; and Acharya J K, Labarca P, Delgado R, Jalink K, Zuker C S. (1998) Synaptic defects and compensatory regulation of inositol metabolism in inositol polyphosphate 1-phosphatase mutants. Neuron 20(6): 1219-29.). It has been demonstrated that therapeutic doses of lithium significantly decrease platelet membrane PIP2 levels in bipolar disorder subjects, providing support to inositol depletion as a mechanism of action in lithium's prophylactic effects (Soares J C, Mallinger A G, Dippold C S, Forster Wells K, Frank E, Kupfer D J. (2000) Effects of lithium on platelet membrane phosphoinositides in bipolar disorder patients: a pilot study. Psychopharmacology (Ber1) 149(1): 12-6.).

Lithium has been demonstrated to inhibit IMPase (Hallcher L M, Sherman W R. (1980) The effects of lithium ion and other agents on the activity of myo-inositol-1-phosphatase from bovine brain. J Biol Chem 255(22): 10896-901.). Two genes encoding human IMPases have been isolated; IMPA1 and IMPA2. Definitive associations of polymorphisms in these genes with response to lithium treatment have not been made. The direct inhibitory effect that lithium has on these gene products which may potentially result in variations in response phenotype, however, has led us to select both gene/SNP sets as inputs for our predictive algorithm.

Lithium has also been demonstrated to inhibit IPPase (Inhorn R C, Majerus P W. (1988) Properties of inositol polyphosphate l-phosphatase. J Biol Chem 263(28): 14559-65.). DNA variations in the human INPP1 gene encoding IPPase have been reported in the coding region. A SNP located in the coding region (base 1276 in NCBI gene document NM₁₃002194; a silent mutation) has been shown to associate with a positive response to lithium in treatment of bipolar disorder (Steen V M, Lovlie R, Osher Y, Belmaker R H, Berle J O, Gulbrandsen A K. (1998) The polymorphic inositol polyphosphate 1-phosphatase gene as a candidate for pharmacogenetic prediction of lithium-responsive manic-depressive illness. Pharmacogenetics 8(3): 259-68.).

Glycogen Synthase Kinase-3beta (GSK-3beta)

Although there are numerous reports indicating that inositol depletion via inhibition of IMPase is a potential mechanism in the action of lithium (see above), there are other reports that debate this hypothesis (see for instance Moorman J M, Leslie R A. (1998) Paradoxical effects of lithium on serotonergic receptor function: an immunocytochemical, behavioural and autoradiographic study. Neuropharmacology 37(3): 357-74.; and Dixon J F, Hokin L E. (1997) The antibipolar drug valproate mimics lithium in stimulating glutamate release and inositol 1,4,5-trisphosphate accumulation in brain cortex slices but not accumulation of inositol monophosphates and bisphosphates. Proc Natl Acad Sci U S A 94(9): 4757-60.). An alternative mechanism of action of lithium in its effect has been proposed to via inhibition of GSK-3beta. GSK3beta is involved in intracellular signaling and is located in the IP3 pathway. GSK3beta has a central role in regulating neuronal plasticity, gene expression, and cell survival, and may be a key component of bipolar disorder, as well as certain other psychiatric diseases (Grimes C A, Jope R S. (2001) The multifaceted roles of glycogen synthase kinase 3beta in cellular signaling. Prog Neurobiol 65(4): 391-426.).

Lithium potently inhibits GSK-3 beta activity. Lithium treatment has been shown to phenocopy loss of GSK-3beta function in both Xenopus and Dictyostelium (Klein P S, Melton D A. (1996) A molecular mechanism for the effect of lithium on development. Proc Natl Acad Sci U S A 93(16): 8455-9.). Inhibition of GSK3beta (which contributes to pro-apoptotic-signaling activity when activated) by lithium has been reported to contribute to anti-apoptotic-signaling mechanisms that result in neuroprotection (see for instance Bijur G N, De Sarno P, Jope R S. (2000) Glycogen synthase kinase-3beta facilitates staurosporine- and heat shock-induced apoptosis. Protection by lithium. J Biol Chem 275(11): 7583-90.; King T D, Bijur G N, Jope RS. (2001) Caspase-3 activation induced by inhibition of mitochondrial complex I is facilitated by glycogen synthase kinase-3beta and attenuated by lithium. Brain Res 919(1): 106-14.; Chalecka-Franaszek E, Chuang D M. (1999) Lithium activates the serine/threonine kinase Akt-1 and suppresses glutamate-induced inhibition of Akt-1 activity in neurons. Proc Natl Acad Sci U S A 96(15): 8745-50.; and Detera-Wadleigh S D. (2001) Lithium-related genetics of bipolar disorder. Ann Med 33(4): 272-85.). Neuroprotection has been recently proposed as a potential mechanism of action in the mid- to long-term alleviation of biopolar symptoms (see for instance Manji H K, Moore G J, Chen G. (2000) Lithium up-regulates the cytoprotective protein Bcl-2 in the CNS in vivo: a role for neurotrophic and neuroprotective effects in manic depressive illness. J Clin Psychiatry 61 Suppl 9(82-96.); and Chen R W, Chuang D M. (1999) Long term lithium treatment suppresses p53 and Bax expression but increases Bcl-2 expression. A prominent role in neuroprotection against excitotoxicity. J Biol Chem 274(10): 6039-42.).

The direct inhibitory effect lithium has on the GSK-3b gene product, and the potential role of inhibition of GSK-3beta in positive response to lithium, has led us to select this gene/SNP set to be inputted into our predictive model.

Bcl-2

Although mood disorders have traditionally been conceptualized as “neurochemical disorders,” considerable literature from a variety of sources demonstrates significant reductions in regional central nervous system (CNS) volume and cell numbers (both neurons and glia) in persons with mood disorders (Manji H K, Moore G J, Chen G. (2000) Lithium up-regulates the cytoprotective protein Bcl-2 in the CNS in vivo: a role for neurotrophic and neuroprotective effects in manic depressive illness. J Clin Psychiatry 61 Suppl 9(82-96.).

Chronic lithium treatment has been demonstrated to markedly increase the levels of the major neuroprotective protein, Bcl-2. Strategies that increase Bcl-2 levels have demonstrated not only protection of neurons against diverse insults (Wei H, Leeds P R, Qian Y, Wei W, Chen R, Chuang D. (2000) beta-amyloid peptide-induced death of PC 12 cells and cerebellar granule cell neurons is inhibited by long-term lithium treatment. Eur J Pharmacol 392(3): 117-23.), but have also demonstrated an increase in the regeneration of CNS axons. These findings suggest that lithium may exert some of its long-term beneficial effects in the treatment of mood disorders via under appreciated neurotrophic and neuroprotective effects. This is also seen with GSK-3beta.

Upregulation of Bcl-2, however, may be due to lithium's effects on GSK-3beta or IMP/IPPases; both which affect PKC—in fact, PKC isozymes have been demonstrated to be reduced under chronic treatment with lithium (Manji H K, Moore G J, Chen G. (2001) Bipolar disorder: leads from the molecular and cellular mechanisms of action of mood stabilizers. Br J Psychiatry Suppl 41(s107-19.)). Attenuated PKC activity has been shown to predispose cells to apoptosis resistance and associate with elevated Bcl-2 levels (Knauf J A, Elisei R, Mochly-Rosen D, Liron T, Chen X N, Gonsky R, Korenberg J R, Fagin J A. (1999) Involvement of protein kinase Cepsilon (PKCepsilon) in thyroid cell death. A truncated chimeric PKCepsilon cloned from a thyroid cancer cell line protects thyroid cells from apoptosis. J Biol Chem 274(33): 23414-25.). Bcl-2, therefore, appears to be indirectly affected by lithium; potentially via the signaling pathways involving PIP2, IP3 and GSK-3beta.

Diagnostic Detection of Mood Disorder-Associated and Treatment-Relevant Mutations:

According to the present invention, base changes in the genes can be detected and used as a diagnostic for Mood disorders. A variety of techniques are available for isolating DNA and RNA and for detecting mutations in the isolated ADRBK2, BNDF, GSK3B, GRK3, IMPA1, IMPA2, INPP1, MARCKS, NTRK2 and/or NR1I2 gene(s).

A number of sample preparation methods are available for isolating DNA and RNA from patient blood samples. For example, the DNA from a blood sample is obtained by cell lysis following alkali treatment. Often, there are multiple copies of RNA message per DNA. Accordingly, it is useful from the standpoint of detection sensitivity to have a sample preparation protocol which isolates both forms of nucleic acid. Total nucleic acid may be isolated by guanidium isothiocyanate/phenol-chloroform extraction, or by proteinase K/phenol-chloroform treatment. Commercially available sample preparation methods such as those from Qiagen Inc. (Chatsworth, Calif.) can also be utilized.

As discussed more fully hereinafter, hybridization with one or more labeled probes containing complements of the variant sequences enables detection of the Mood disorders mutations. Since each Mood disorders patient can be heteroplasmic (possessing both the Mood disorders mutation and the normal sequence) a quantitative or semi-quantitative measure (depending on the detection method) of such heteroplasmy can be obtained by comparing the amount of signal from the Mood disorders probe to the amount from the Mood disorders.sup.-(normal or wild-type) probe.

A variety of techniques, as discussed more fully hereinafter, are available for detecting the specific mutations in the ADRBK2, BNDF, GSK3B, GRK3, IMPA1, IMPA2, INPP1, MARCKS, NTRK2 and/or NR1I2 gene(s). The detection methods include, for example, cloning and sequencing, ligation of oligonucleotides, use of the polymerase chain reaction and variations thereof, use of single nucleotide primer-guided extension assays, hybridization techniques using target-specific oligonucleotides and sandwich hybridization methods.

Cloning and sequencing of the ADRBK2, BNDF, GSK3B, GRK3, IMPA1, IMPA2, INPP1, MARCKS, NTRK2 and/or NR1I2 gene(s) can serve to detect Mood disorders mutations in patient samples. Sequencing can be carried out with commercially available automated sequencers utilizing fluorescently labeled primers. An alternate sequencing strategy is the “sequencing by hybridization” method using high density oligonucleotide arrays on silicon chips (Fodor et al., Nature 364:555-556 (1993); Pease et al., Proc. Natl. Acad. Sci. USA, 91:5022-5026 (1994). For example, fluorescently-labeled target nucleic acid generated, for example from PCR amplification of the target genes using fluorescently labeled primers, are hybridized with a chip containing a set of short oligonucleotides which probe regions of complementarity with the target sequence. The resulting hybridization patterns are useful for reassembling the original target DNA sequence.

Mutational analysis can also be carried out by methods based on ligation of oligonucleotide sequences which anneal immediately adjacent to each other on a target DNA or RNA molecule (Wu and Wallace, Genomics 4:560-569 (1989); Landren et al., Science 241:1077-1080 (1988); Nickerson et al., Proc. Natl. Acad. Sci. 87:8923-8927 (1990); Barany, F., Proc. Natl. Acad. Sci. 88:189-193 (1991)). Ligase-mediated covalent attachment occurs only when the oligonucleotides are correctly base-paired. The Ligase Chain Reaction (LCR), which utilizes the thermostable Taq ligase for target amplification, is particularly useful for interrogating Mood disorders mutation loci. The elevated reaction temperatures permits the ligation reaction to be conducted with high stringency (Barany, F., PCR Methods and Applications 1:5-16 (1991)).

Analysis of point mutations in DNA can also be carried out by using the polymerase chain reaction (PCR) and variations thereof. Mismatches can be detected by competitive oligonucleotide priming under hybridization conditions where binding of the perfectly matched primer is favored (Gibbs et al., Nucl. Acids. Res. 17:2437-2448 (1989)). In the amplification refractory mutation system technique (ARMS), primers are designed to have perfect matches or mismatches with target sequences either internal or at the 3′ residue (Newton et al., Nucl. Acids. Res. 17:2503-2516 (1989)). Under appropriate conditions, only the perfectly annealed oligonucleotide functions as a primer for the PCR reaction, thus providing a method of discrimination between normal and mutant (Mood disorders) sequences.

Genotyping analysis of the adrenergic beta receptor kinase 2 (ADRBK2), the gene encoding the polypeptide brain-derived neurotrophic factor (BNDF), Glycogen synthase kinase-3 beta (GSK3B), G protein-coupled receptor kinase 3 (GRK3), Inositol (myo)-1(or 4)-monophosphatase 1 (IMPA1), Inositol (myo)-1(or 4)-monophosphatase 2 (IMPA2), Inositol polyphosphate 1-phosphatase (INPP1), Myristoylated alanine-rich C-kinase substrate (MARCKS), BDNF/NT-3 growth factors receptor precursor (NTRK2), and/or Orphan nuclear receptor PXR (NR1I2) gene(s) can also be carried out using single nucleotide primer-guided extension assays, where the specific incorporation of the correct base is provided by the high fidelity of the DNA polymerase (Syvanen et al., Genomics 8:684-692 (1990); Kuppuswamy et al., Proc. Natl. Acad. Sci. USA. 88:1143-1147 (1991)). Another primer extension assay, which allows for the quantification of heteroplasmy by simultaneously interrogating both wild-type and mutant nucleotides, is disclosed in a pending U.S. patent application entitled, “Multiplexed Primer Extension Methods”, naming Eoin Fahy and Soumitra Ghosh as inventors, filed on Mar. 24, 1995, U.S. Ser. No. 08/410,658, the disclosure of which is incorporated by reference.

Detection of single base mutations in target nucleic acids can be conveniently accomplished by differential hybridization techniques using target-specific oligonucleotides (Suggs et al., Proc. Natl. Acad. Sci. 78:6613-6617 (1981); Conner et al., Proc. Natl. Acad. Sci. 80:278-282 (1983); Saiki et al., Proc. Natl. Acad. Sci. 86:6230-6234 (1989)). For example, mutations are diagnosed on the basis of the higher thermal stability of the perfectly matched probes as compared to the mismatched probes. The hybridization reactions may be carried out in a filter-based format, in which the target nucleic acids are immobilized on nitrocellulose or nylon membranes and probed with oligonucleotide probes. Any of the known hybridization formats may be used, including Southern blots, slot blots, “reverse” dot blots, solution hybridization, solid support based sandwich hybridization, bead-based, silicon chip-based and microtiter well-based hybridization formats.

An alternative strategy involves detection of the ADRBK2, BNDF, GSK3B, GRK3, IMPA1, IMPA2, INPP1, MARCKS, NTRK2 and/or NR1I2 gene(s) by sandwich hybridization methods. In this strategy, the mutant and wild-type (normal) target nucleic acids are separated from non-homologous DNA/RNA using a common capture oligonucleotide immobilized on a solid support and detected by specific oligonucleotide probes tagged with reporter labels. The capture oligonucleotides can be immobilized on microtitre plate wells or on beads (Gingeras et al., J. Infect. Dis. 164:1066-1074 (1991); Richman et al., Proc. Natl. Acad. Sci. 88:11241-11245 (1991)).

While radio-isotopic labeled detection oligonucleotide probes are highly sensitive, non-isotopic labels are preferred due to concerns about handling and disposal of radioactivity. A number of strategies are available for detecting target nucleic acids by non-isotopic means (Matthews et al., Anal. Biochem., 169:1-25 (1988)). The non-isotopic detection method may be direct or indirect.

The indirect detection process is generally where the oligonucleotide probe is covalently labeled with a hapten or ligand such as digoxigenin (DIG) or biotin. Following the hybridization step, the target-probe duplex is detected by an antibody- or streptavidin-enzyme complex. Enzymes commonly used in DNA diagnostics are horseradish peroxidase and alkaline phosphatase. One particular indirect method, the Genius.TM. detection system (Boehringer Mannheim) is especially useful for mutational analysis of the ADRBK2, BNDF, GSK3B, GRK3, IMPA1, IMPA2, INPP1, MARCKS, NTRK2 and/or NR1I2 gene(s). This indirect method uses digoxigenin as the tag for the oligonucleotide probe and is detected by an anti-digoxigenin-antibody-alkaline phosphatase conjugate.

Direct detection methods include the use of fluorophor-labeled oligonucleotides, lanthanide chelate-labeled oligonucleotides or oligonucleotide-enzyme conjugates. Examples of fluorophor labels are fluorescein, rhodamine and phthalocyanine dyes. Examples of lanthanide chelates include complexes of Eu.sup.3+ and Th.sup.3+. Directly labeled oligonucleotide-enzyme conjugates are preferred for detecting point mutations when using target-specific oligonucleotides as they provide very high sensitivities of detection.

Oligonucleotide-enzyme conjugates can be prepared by a number of methods (Jablonski et al., Nucl. Acids Res., 14:6115-6128 (1986); Li et al., Nucl. Acids Res. 15:5275-5287 (1987); Ghosh et al., Bioconjugate Chem. 1:71-76 (1990)), and alkaline phosphatase is the enzyme of choice for obtaining high sensitivities of detection. The detection of target nucleic acids using these conjugates can be carried out by filter hybridization methods or by bead-based sandwich hybridization (Ishii et al., Bioconjugate Chemistry 4:34-41 (1993)).

Detection of the probe label may be accomplished by the following approaches. For radioisotopes, detection is by autoradiography, scintillation counting or phosphor imaging. For hapten or biotin labels, detection is with antibody or streptavidin bound to a reporter enzyme such as horseradish peroxidase or alkaline phosphatase, which is then detected by enzymatic means. For fluorophor or lanthanide-chelate labels, fluorescent signals may be measured with spectrofluorimeters with or without time-resolved mode or using automated microtitre plate readers. With enzyme labels, detection is by color or dye deposition (p-nitropheny phosphate or 5-bromo-4-chloro-3-indolyl phosphate/nitroblue tetrazolium for alkaline phosphatase and 3,3′-diaminobenzidine-NiCl.sub.2 for horseradish peroxidase), fluorescence (e.g., 4-methyl umbelliferyl phosphate for alkaline phosphatase) or chemiluminescence (the alkaline phosphatase dioxetane substrates LumiPhos 530 from Lumigen Inc., Detroit Mich. or AMPPD and CSPD from Tropix, Inc.). Chemiluminescent detection may be carried out with X-ray or polaroid film or by using single photon counting luminometers. This is the preferred detection format for alkaline phosphatase labelled probes.

The oligonucleotide probes for detection preferably range in size between 10 and 100 bases, more preferably between 15 and 30 bases in length. In order to obtain the required target discrimination using the detection oligonucleotide probes, the hybridization reactions are preferably run between 20.degree. C. and 60.degree. C., and more preferably between 30.degree. C. and 55.degree. C. As known to those skilled in the art, optimal discrimination between perfect and mismatched duplexes can be obtained by manipulating the temperature and/or salt concentrations or inclusion of formamide in the stringency washes.

Pastinen et al. (1997), incorporated herein by reference, describe a method for multiplex detection of single nucleotide polymorphism in which the solid phase minisequencing principle is applied to an oligonucleotide array format. High-density arrays of DNA probes attached to a solid support (DNA chips) are further described in herein.

In one aspect the present invention provides polynucleotides and methods to genotype one or more biallelic markers of the present invention by performing a microsequencing assay. It will be appreciated any primer having a 3′ end immediately adjacent to a polymorphic nucleotide may be used. Similarly, it will be appreciated that microsequencing analysis may be performed for any biallelic marker or any combination of biallelic markers of the present invention. One aspect of the present invention is a solid support which includes one or more microsequencing primers for the SNPs listed in APPENDIX I, or fragments comprising at least 8, at least 12, at least 15, or at least 20 consecutive nucleotides thereof and having a 3′ terminus immediately upstream of the corresponding biallelic marker, for determining the identity of a nucleotide at biallelic marker site.

Hybridization to Addressable Arrays of Oligonucleotides [0219] Hybridization assays based on oligonucleotide arrays rely on the differences in hybridization stability of short oligonucleotides to perfectly matched and mismatched target sequence variants. Efficient access to polymorphism information is obtained through a basic structure comprising high-density arrays of oligonucleotide probes attached to a solid support (the chip) at selected positions. Each DNA chip can contain thousands to millions of individual synthetic DNA probes arranged in a grid-like pattern and miniaturized to the size of a dime or smaller.

The chip technology has already been applied with success in numerous cases. For example, the screening of mutations has been undertaken in the BRCA1 gene, in S. cerevisiae mutant strains, and in the protease gene of HIV-1 virus (Hacia et al., 1996; Shoemaker et al., 1996; Kozal et al., 1996, the disclosclosures of which are incorporated herein by reference). Chips of various formats for use in detecting biallelic polymorphisms can be produced on a customized basis by Affymetrix (GeneChip.TM.), Hyseq (HyChip and HyGnostics), and Protogene Laboratories.

In general, these methods employ arrays of oligonucleotide probes that are complementary to target nucleic acid sequence segments from an individual which, target sequences include a polymorphic marker. EP785280, the disclosures of which is incorporated herein by reference in its entirety, describes a tiling strategy for the detection of single nucleotide polymorphisms. Briefly, arrays may generally be “tiled” for a large number of specific polymorphisms. By “tiling” is generally meant the synthesis of a defined set of oligonucleotide probes which is made up of a sequence complementary to the target sequence of interest, as well as preselected variations of that sequence, e.g., substitution of one or more given positions with one or more members of the basis set of monomers, i.e. nucleotides. Tiling strategies are further described in PCT application No. WO 95/11995, incorporated herein by reference. In a particular aspect, arrays are tiled for a number of specific, identified biallelic marker sequences. In particular the array is tiled to include a number of detection blocks, each detection block being specific for a specific biallelic marker or a set of biallelic markers. For example, a detection block may be tiled to include a number of probes, which span the sequence segment that includes a specific polymorphism. To ensure probes that are complementary to each allele, the probes are synthesized in pairs differing at the biallelic marker. In addition to the probes differing at the polymorphic base, monosubstituted probes are also generally tiled within the detection block. These monosubstituted probes have bases at and up to a certain number of bases in either direction from the polymorphism, substituted with the remaining nucleotides (selected from A, T, G, C and U). Typically the probes in a tiled detection block will include substitutions of the sequence positions up to and including those that are 5 bases away from the biallelic marker. The monosubstituted probes provide internal controls for the tiled array, to distinguish actual hybridization from artefactual cross-hybridization. Upon completion of hybridization with the target sequence and washing of the array, the array is scanned to determine the position on the array to which the target sequence hybridizes. The hybridization data from the scanned array is then analyzed to identify which allele or alleles of the biallelic marker are present in the sample. Hybridization and scanning may be carried out as described in PCT application No. WO 92/10092 and WO 95/11995 and U.S. Pat. No. 5,424,186, the disclosures of which are incorporated herein by reference.

Thus, in some embodiments, the chips may comprise an array of nucleic acid sequences of fragments of about 15 nucleotides in length. In further embodiments, the chip may comprise an array including at least one of the sequences selected from the group consisting of sequences corresponding to the nucleotides detailed in APPENDIX I, and the sequences complementary thereto, or a fragment thereof at least about 8 consecutive nucleotides, preferably 10, 15, 20, more preferably 25, 30, 40, 47, or 50 consecutive nucleotides. In some embodiments, the chip may comprise an array of at least 2, 3, 4, 5, 6, 7, 8 or more of these polynucleotides of the invention. Solid supports and polynucleotides of the present invention attached to solid supports are further described in the section titled “Oligonucleotide probes and Primers”.

Integrated Systems

Another technique, which may be used to analyze polymorphisms, includes multicomponent integrated systems, which miniaturize and compartmentalize processes such as PCR and capillary electrophoresis reactions in a single functional device. An example of such technique is disclosed in U.S. Pat. No. 5,589,136, the disclosure of which is incorporated herein by reference in its entirety, which describes the integration of PCR amplification and capillary electrophoresis in chips.

Integrated systems can be envisaged mainly when microfluidic systems are used. These systems comprise a pattern of microchannels designed onto a glass, silicon, quartz, or plastic wafer included on a microchip. The movements of the samples are controlled by electric, electroosmotic or hydrostatic forces applied across different areas of the microchip. For genotyping biallelic markers, the microfluidic system may integrate nucleic acid amplification, microsequencing, capillary electrophoresis and a detection method such as laser-induced fluorescence detection.

As an alternative to detection of mutations in the nucleic acids associated with the ADRBK2, BNDF, GSK3B, GRK3, IMPA1, IMPA2, INPP1, MARCKS, NTRK2 and/or NR1I2 gene(s), it is also possible to analyze the protein products of the ADRBK2, BNDF, GSK3B, GRK3, IMPA1, IMPA2, INPP1, MARCKS, NTRK2 and/or NR1I2 gene(s). In particular, point mutations in these genes are expected to alter the structure of the proteins for which these gene encode. These altered proteins (variant polypeptides) can be isolated and used to prepare antisera and monoclonal antibodies that specifically detect the products of the mutated genes and not those of non-mutated or wild-type genes. Mutated gene products also can be used to immunize animals for the production of polyclonal antibodies. Recombinantly produced peptides can also be used to generate polyclonal antibodies. These peptides may represent small fragments of gene products produced by expressing regions of the mitochondrial genome containing point mutations.

More particularly, variant polypeptides from point mutations in said genes can be used to immunize an animal for the production of polyclonal antiserum. For example, a recombinantly produced fragment of a variant polypeptide can be injected into a mouse along with an adjuvant so as to generate an immune response. Murine immunoglobulins which bind the recombinant fragment with a binding affinity of at least 1.times.10.sup.7 M.sup.-1 can be harvested from the immunized mouse as an antiserum, and may be further purified by affinity chromatography or other means. Additionally, spleen cells are harvested from the mouse and fused to myeloma cells to produce a bank of antibody-secreting hybridoma cells. The bank of hybridomas can be screened for clones that secrete immunoglobulins which bind the recombinantly produced fragment with an affinity of at least 1.times.10.sup.6 M.sup.-1. More specifically, immunoglobulins that selectively bind to the variant polypeptides but poorly or not at all to wild-type polypeptides are selected, either by pre-absorption with wild-type proteins or by screening of hybridoma cell lines for specific idiotypes that bind the variant, but not wild-type, polypeptides.

Nucleic acid sequences capable of ultimately expressing the desired variant polypeptides can be formed from a variety of different polynucleotides (genomic or cDNA, RNA, synthetic oligonucleotides, etc.) as well as by a variety of different techniques.

The DNA sequences can be expressed in hosts after the sequences have been operably linked to (i.e., positioned to ensure the functioning of) an expression control sequence. These expression vectors are typically replicable in the host organisms either as episomes or as an integral part of the host chromosomal DNA. Commonly, expression vectors can contain selection markers (e.g., markers based on tetracyclinic resistance or hygromycin resistance) to permit detection and/or selection of those cells transformed with the desired DNA sequences. Further details can be found in U.S. Pat. No. 4,704,362.

Polynucleotides encoding a variant polypeptide may include sequences that facilitate transcription (expression sequences) and translation of the coding sequences such that the encoded polypeptide product is produced. Construction of such polynucleotides is well known in the art. For example, such polynucleotides can include a promoter, a transcription termination site (polyadenylation site in eukaryotic expression hosts), a ribosome binding site, and, optionally, an enhancer for use in eukaryotic expression hosts, and, optionally, sequences necessary for replication of a vector.

E. coli is one prokaryotic host useful particularly for cloning DNA sequences of the present invention. Other microbial hosts suitable for use include bacilli, such as Bacillus subtilus, and other enterobacteriaceae, such as Salmonella, Serratia, and various Pseudomonas species. In these prokaryotic hosts one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. The promoters will typically control expression, optionally with an operator sequence, and have ribosome binding site sequences, for example, for initiating and completing transcription and translation.

Other microbes, such as yeast, may also be used for expression. Saccharomyces can be a suitable host, with suitable vectors having expression control sequences, such as promoters, including 3-phosphoglycerate kinase or other glycolytic enzymes, and an origin of replication, termination sequences, etc. as desired.

In addition to microorganisms, mammalian tissue cell culture may also be used to express and produce the polypeptides of the present invention. Eukaryotic cells are actually preferred, because a number of suitable host cell lines capable of secreting intact human proteins have been developed in the art, and include the CHO cell lines, various COS cell lines, HeLa cells, myeloma cell lines, Jurkat cells, and so forth. Expression vectors for these cells can include expression control sequences, such as an origin of replication, a promoter, an enhancer, an necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, and so forth. The vectors containing the DNA segments of interest (e.g., polypeptides encoding a variant polypeptide) can be transferred into the host cell by well-known methods, which vary depending on the type of cellular host. For example, calcium chloride transfection is commonly utilized for prokaryotic cells, whereas calcium phosphate treatment or electroporation may be used for other cellular hosts.

The method lends itself readily to the formulation of test kits for use in diagnosis. Such a kit would comprise a carrier compartmentalized to receive in close confinement one or more containers wherein a first container may contain suitably labeled DNA or immunological probes. Other containers may contain reagents useful in the localization of the labeled probes, such as enzyme substrates. Still other containers may contain restriction enzymes, buffers etc., together with instructions for use.

Linkage Disequilibrium Analysis

Linkage disequilibrium is the non-random association of alleles at two or more loci and represents a powerful tool for mapping genes involved in disease traits (see Ajioka R. S. et al., 1997, incorporated herein by reference). Biallelic markers, because they are densely spaced in the human genome and can be genotyped in more numerous numbers than other types of genetic markers (such as RFLP or VNTR markers), are particularly useful in genetic analysis based on linkage disequilibrium. The biallelic markers of the present invention may be used in any linkage disequilibrium analysis method known in the art.

Briefly, when a disease mutation is first introduced into a population (by a new mutation or the immigration of a mutation carrier), it necessarily resides on a single chromosome and thus on a single “background” or “ancestral” haplotype of linked markers. Consequently, there is complete disequilibrium between these markers and the disease mutation: one finds the disease mutation only in the presence of a specific set of marker alleles. Through subsequent generations recombinations occur between the disease mutation and these marker polymorphisms, and the disequilibrium gradually dissipates. The pace of this dissipation is a function of the recombination frequency, so the markers closest to the disease gene will manifest higher levels of disequilibrium than those that are further away. When not broken up by recombination, “ancestral” haplotypes and linkage disequilibrium between marker alleles at different loci can be tracked not only through pedigrees but also through populations. Linkage disequilibrium is usually seen as an association between one specific allele at one locus and another specific allele at a second locus.

The pattern or curve of disequilibrium between disease and marker loci is expected to exhibit a maximum that occurs at the disease locus. Consequently, the amount of linkage disequilibrium between a disease allele and closely linked genetic markers may yield valuable information regarding the location of the disease gene. For fine-scale mapping of a disease locus, it is useful to have some knowledge of the patterns of linkage disequilibrium that exist between markers in the studied region. As mentioned above the mapping resolution achieved through the analysis of linkage disequilibrium is much higher than that of linkage studies. The high density of biallelic markers combined with linkage disequilibrium analysis provides powerful tools for fine-scale mapping. Different methods to calculate linkage disequilibrium are described below under the heading “Statistical Methods”.

Methods to Calculate Linkage Disequilibrium Between Markers

A number of methods can be used to calculate linkage disequilibrium between any two genetic positions, in practice linkage disequilibrium is measured by applying a statistical association test to haplotype data taken from a population. Linkage disequilibrium between any pair of biallelic markers comprising at least one of the biallelic markers of the present invention (M.sub.i, M.sub.j) having alleles (a.sub.i/b.sub.i) at marker M.sub.i and alleles (a.sub.j/b.sub.j) at marker M.sub.j can be calculated for every allele combination (a.sub.i,a.sub.j; a.sub.i,b.sub.j; b.sub.i,a.sub.j and b.sub.i,b.sub.j), according to the Piazza formula: .DELTA..sub.aiaj={squareroot}.theta.4−{squareroot}(.theta.4+.theta.3) (.theta.4+.theta.2), where:
.theta.4=−−=frequency of genotypes not having allele a.sub.i at M.sub.i and not having allele a.sub.j at M.sub.j
.theta.3=−+=frequency of genotypes not having allele a.sub.i at M.sub.i and having allele a.sub.j at M.sub.j
.theta.2=+−=frequency of genotypes having allele a.sub.i at M.sub.i and not having allele a.sub.j at M.sub.j

Linkage disequilibrium (LD) between pairs of biallelic markers (M.sub.i, M.sub.j) can also be calculated for every allele combination (ai,aj; ai,bj; b.sub.i,a.sub.j and b.sub.i,b.sub.j), according to the maximum-likelihood estimate (MLE) for delta (the composite genotypic disequilibrium coefficient), as described by Weir (Weir B. S., 1996). The MLE for the composite linkage disequilibrium is: D.sub.aiaj=(2n.sub.1+n.sub.2+n.sub.3+n.sub.4/2)/N-2(pr(a.sub.i).pr(a.sub.j−)) where
n.sub.1=.SIGMA. phenotype (a.sub.i/a.sub.i, a.sub.j/a.sub.j), n.sub.2=.SIGMA. phenotype (a.sub.i/a.sub.i, a.sub.j/b.sub.j), n.sub.3=.SIGMA. phenotype (a.sub.i/b.sub.i, a.sub.j/a.sub.j), n4=.SIGMA. phenotype (a.sub.i/b.sub.i, a.sub.j/b.sub.j) and N is the number of individuals in the sample. This formula allows linkage disequilibrium between alleles to be estimated when only genotype, and not haplotype, data are available.

Another means of calculating the linkage disequilibrium between markers is as follows. For a couple of biallelic markers, M.sub.i(a.sub.i/b.sub.i) and M.sub.j(a.sub.j/b.sub.j), fitting the Hardy-Weinberg equilibrium, one can estimate the four possible haplotype frequencies in a given population according to the approach described above.

The estimation of gametic disequilibrium between ai and aj is simply: 5 D aiaj=pr(haplotype(a i, a j ))−pr(a i)pr(a j).

Where pr(a.sub.i) is the probability of allele a.sub.i and pr(a.sub.j) is the probability of allele a.sub.j and where pr(haplotype (a.sub.i, a.sub.j)) is estimated as in Equation 3 above.

For a couple of biallelic marker only one measure of disequilibrium is necessary to describe the association between M.sub.i and M.sub.j.

Then a normalised value of the above is calculated as follows: 6 D aiaj′=D aiaj/max(−pr(a i) pr(a j), −pr(b i)pr(b j )) with D aiaj<0D aiaj′=D aiaj/max(pr(b i)pr(a j), pr(a i) pr(b j)) with D aiaj>0.

The skilled person will readily appreciate that other LD calculation methods can be used without undue experimentation.

Identification of Biallelic Markers in Linkage Disequilibrium with the Biallelic Markers of the Invention

Once a first biallelic marker has been identified in a genomic region of interest, the practitioner of ordinary skill in the art, using the teachings of the present invention, can easily identify additional biallelic markers in linkage disequilibrium with this first marker. As mentioned before, any marker in linkage disequilibrium with a first marker associated with a trait will be associated with the trait. Therefore, once an association has been demonstrated between a given biallelic marker and a trait, the discovery of additional biallelic markers associated with this trait is of great interest in order to increase the density of biallelic markers in this particular region. The causal gene or mutation will be found in the vicinity of the marker or set of markers showing the highest correlation with the trait.

Identification of additional markers in linkage disequilibrium with a given marker involves: (a) amplifying a genomic fragment comprising a first biallelic marker from a plurality of individuals; (b) identifying of second biallelic markers in the genomic region harboring said first biallelic marker; (c) conducting a linkage disequilibrium analysis between said first biallelic marker and second biallelic markers; and (d) selecting said second biallelic markers as being in linkage disequilibrium with said first marker. Subcombinations comprising steps (b) and (c) are also contemplated.

Methods to identify biallelic markers and to conduct linkage disequilibrium analysis are described herein and can be carried out by the skilled person without undue experimentation. The present invention then also concerns biallelic markers and other polymorphisms which are in linkage disequilibrium with the specific biallelic markers of the invention and which are expected to present similar characteristics in terms of their respective association with a given trait. In a preferred embodiment, the invnetion concerns biallelic markers which are in linkage disequilibrium with the specific biallelic markers.

Assays for Identification of Compounds for Treatment of Mood Disorders

The present invention provides assays which may be used to test compounds for their ability to treat mood disorders, and in particular, to ameliorate symptoms of a mood disorder mediated by ADRBK2, BNDF, GSK3B, GRK3, IMPA1, IMPA2, INPP1, MARCKS, NTRK2 and/or NR1I2, g34665, sbg2, g35017 or g35018. In preferred embodiments, compounds tested for their ability to ameliorate syptoms of mood disorders mediated by ADRBK2, BNDF, GSK3B, GRK3, IMPA1, IMPA2, INPP1, MARCKS, NTRK2 and/or NR1I2 , g34665, sbg2, g35017 or g35018. Compounds may also be tested for their ability to treat related disorders, including among others psychotic disorders, mood disorders, autism, substance dependence and alcoholism, mental retardation, and other psychiatric diseases including cognitive, anxiety, eating, impulse-control, and personality disorders, as defined with DSM-IV classification.

The present invention also provides cell and animal, including primate and mouse, models of schizophrenia, bipolar disorder and related disorders.

In one aspect, provided are non-cell based, cell based and animal based assays for the identification of such compounds that affect ADRBK2, BNDF, GSK3B, GRK3, IMPA1, IMPA2, INPP1, MARCKS, NTRK2 and/or NR1I2 activity. ADRBK2, BNDF, GSK3B, GRK3, IMPA1, IMPA2, INPP1, MARCKS, NTRK2 and/or NR1I2 activity may be affected by any mechanism; in certain embodiments, ADRBK2, BNDF, GSK3B, GRK3, IMPA1, IMPA2, INPP1, MARCKS, NTRK2 and/or NR1I2 activity is affected by modulating ADRBK2, BNDF, GSK3B, GRK3, IMPA1, IMPA2, INPP1, MARCKS, NTRK2 and/or NR1I2 gene expression or the activity of the ADRBK2, BNDF, GSK3B, GRK3, IMPA1, IMPA2, INPP1, MARCKS, NTRK2 and/or NR1I2 gene product.

The present methods allow the identification of compounds that affect ADRBK2, BNDF, GSK3B, GRK3, IMPA1, IMPA2, INPP1, MARCKS, NTRK2 and/or NR1I2 activity directly or indirectly. Thus, the non-cell based, cell based and animal assays of the present invention may also be used to identify compounds that act on an element of a ADRBK2, BNDF, GSK3B, GRK3, IMPA1, IMPA2, INPP1, MARCKS, NTRK2 and/or NR1I2 pathway other than ADRBK2, BNDF, GSK3B, GRK3, IMPA1, IMPA2, INPP1, MARCKS, NTRK2 and/or NR1I2 itself. These compounds can then be used as a therapeutic treatment to modulate ADRBK2, BNDF, GSK3B, GRK3, IMPA1, IMPA2, INPP1, MARCKS, NTRK2 and/or NR1I2 and other gene products involved in schizophrenia, bipolar disorder and related disorders.

Cell and Non-Cell Based Assays

In one aspect, cell based assays using recombinant or non-recombinant cells may be used to identify compounds which modulate ADRBK2, BNDF, GSK3B, GRK3, IMPA1, IMPA2, INPP1, MARCKS, NTRK2 and/or NR1I2 activity.

In one aspect, a cell based assay of the invention encompasses a method for identifying a test compound for the treatment of schizophrenia or bipolar disorder comprising (a) exposing a cell to a test compound at a concentration and time sufficient to ameliorate an endpoint related to schizophrenia or bipolar disorder, and (b) determining the level of ADRBK2, BNDF, GSK3B, GRK3, IMPA1, IMPA2, INPP1, MARCKS, NTRK2 and/or NR1I2 activity in a cell. ADRBK2, BNDF, GSK3B, GRK3, IMPA1, IMPA2, INPP1, MARCKS, NTRK2 and/or NR1I2 activity can be measured, for example, by assaying a cell for mRNA transcript level, ADRBK2, BNDF, GSK3B, GRK3, IMPA1, IMPA2, INPP1, MARCKS, NTRK2 and/or NR1I2 peptide expression, localization or protein activity. Preferably the test compound is a compound capable of or suspected to be capable of ameliorating a symptom of schizophrenia, bipolar disorder or a related disorder. Test compounds capable of modulating ADRBK2, BNDF, GSK3B, GRK3, IMPA1, IMPA2, INPP1, MARCKS, NTRK2 and/or NR1I2 activity may be selected for use in developing therapeutics.

In another aspect, a cell based assay of the invention encompasses a method for identifying a compound for the treatment of schizophrenia or bipolar disorder comprising (a) exposing a cell to a level of ADRBK2, BNDF, GSK3B, GRK3, IMPA1, IMPA2, INPP1, MARCKS, NTRK2 and/or NR1I2 activity sufficient to cause a schizophrenia-related or bipolar disorder-related endpoint, and (b) exposing said cell to a test compound. A test compound can then be selected according to its ability to ameliorate said schizophrenia-related or bipolar disorder-related endpoints. ADRBK2, BNDF, GSK3B, GRK3, IMPA1, IMPA2, INPP1, MARCKS, NTRK2 and/or NR1I2 activity may be provided by any suitable method, including but not limited to providing a vector containing an ADRBK2, BNDF, GSK3B, GRK3, IMPA1, IMPA2, INPP1, MARCKS, NTRK2 and/or NR1I2 nucleotide sequence, treating said cell with a compound capable of increasing ADRBK2, BNDF, GSK3B, GRK3, IMPA1, IMPA2, INPP1, MARCKS, NTRK2 and/or NR1I2 expression and treating said cell with an ADRBK2, BNDF, GSK3B, GRK3, IMPA1, IMPA2, INPP1, MARCKS, NTRK2 and/or NR1I2 peptide. Preferably said cell is treated with an ADRBK2, BNDF, GSK3B, GRK3, IMPA1, IMPA2, INPP1, MARCKS, NTRK2 and/or NR1I2 peptide comprising a contiguous span of at least 4 amino acids of sequences corresponding to the SNPs of APPENDIX I. Preferably the test compound is a compound capable of or suspected to be capable of ameliorating a symtpom of schizophrenia, bipolar disorder or a related disorder; alternatively, the test compound is suspected of exacerbating an endpoint schizophrenia, bipolar disorder or a related disorder. A test compound capable of ameliorating any detectable symptom or endpoint of a schizophrenia, bipolar disorder or a related disorder may be selected for use in developing medicaments.

In another embodiment, the invention provides cell and non-cell based assays to ADRBK2, BNDF, GSK3B, GRK3, IMPA1, IMPA2, INPP1, MARCKS, NTRK2 and/or NR1I2 to determine whether sbg peptides bind to the cell surface, and to identify compounds for the treatment of schizophrenia, bipolar disorder and related disorders that interact with an ADRBK2, BNDF, GSK3B, GRK3, IMPA1, IMPA2, INPP1, MARCKS, NTRK2 and/or NR1I12 receptor. In one such embodiment, an ADRBK2, BNDF, GSK3B, GRK3, IMPA1, IMPA2, INPP1, MARCKS, NTRK2 and/or NR1I2 polynucleotide, or fragments thereof, is cloned into expression vectors such as those described herein. The proteins are purified by size, charge, immunochromatography or other techniques familiar to those skilled in the art. Following purification, the proteins are labeled using techniques known to those skilled in the art. The labeled proteins are incubated with cells or cell lines derived from a variety of organs or tissues to allow the proteins to bind to any receptor present on the cell surface. Following the incubation, the cells are washed to remove non-specifically bound protein. The labeled proteins are detected by autoradiography. Alternatively, unlabeled proteins may be incubated with the cells and detected with antibodies having a detectable label, such as a fluorescent molecule, attached thereto. Specificity of cell surface binding may be analyzed by conducting a competition analysis in which various amounts of unlabeled protein are incubated along with the labeled protein. The amount of labeled protein bound to the cell surface decreases as the amount of competitive unlabeled protein increases. As a control, various amounts of an unlabeled protein unrelated to the labeled protein is included in some binding reactions. The amount of labeled protein bound to the cell surface does not decrease in binding reactions containing increasing amounts of unrelated unlabeled protein, indicating that the protein encoded by the nucleic acid binds specifically to the cell surface.

In another embodiment, the present invention comprises non-cell based binding assays, wherein an ADRBK2, BNDF, GSK3B, GRK3, IMPA1, IMPA2, INPP1, MARCKS, NTRK2 and/or NR1I2 polypeptide is prepared and purified as in cell based binding assays described above. Following purification, the proteins are labeled and incubated with a cell membrane extract or isolate derived from any desired cells from any organs, tissue or combination of organs or tissues of interest to allow the ADRBK2, BNDF, GSK3B, GRK3, IMPA1, IMPA2, INPP1, MARCKS, NTRK2 and/or NR1I2 polypeptide to bind to any receptor present on a membrane. Following the incubation, the membranes are washed to remove non-specifically bound protein. The labeled proteins may be detected by autoradiography. Specificity of membrane binding of ADRBK2, BNDF, GSK3B, GRK3, IMPA1, IMPA2, INPP1, MARCKS, NTRK2 and/or NR1I2 may be analyzed by conducting a competition analysis in which various amounts of a test compound are incubated along with the labeled protein. Any desired test compound, including test polypeptides, can be incubated with the cells. The test compounds may be detected with antibodies having a detectable label, such as a fluorescent molecule, attached thereto. The amount of labeled ADRBK2, BNDF, GSK3B, GRK3, IMPA1, IMPA2, INPP1, MARCKS, NTRK2 and/or NR1I2 polypeptide bound to the cell surface decreases as the amount of competitive test compound increases. As a control, various amounts of an unlabeled protein or a compound unrelated to the test compound is included in some binding reactions. Test compounds capable of reducing the amount of ADRBK2, BNDF, GSK3B, GRK3, IMPA1, IMPA2, INPP1, MARCKS, NTRK2 and/or NR1I2 bound to cell membranes may be selected as a candidate therapeutic compound.

In preferred embodiments of the cell and non-cell based assays, said ADRBK2, BNDF, GSK3B, GRK3, IMPA1, IMPA2, INPP1, MARCKS, NTRK2 and/or NR1I2 peptide comprising a contiguous span of at least 4 amino acids of sequences corresponding to the SNPs of APPENDIX I.

Said cell based assays may comprise cells of any suitable origin; particularly preferred cells are human cells, primate cells, non-human primate cells and mouse cells. If non-human primate cells are used, the ADRBK2, BNDF, GSK3B, GRK3, IMPA1, IMPA2, INPP1, MARCKS, NTRK2 and/or NR1I2 may comprise a nucleotide sequence or be encoded by a nucleotide sequence according to the primate nucleic acid sequences of sequences corresponding to the SNPs of APPENDIX I, or a sequence complementary thereto or a fragment thereof.

Animal Model Based Assay

Non-human animal-based assays may also be used to identify compounds which modulate ADRBK2, BNDF, GSK3B, GRK3, IMPA1, IMPA2, INPP1, MARCKS, NTRK2 and/or NR1I2 activity. Thus, the present invention comprises treating an animal affected by a mood disorder or symptoms thereof with a test compound capable of directly or indirectly modulating ADRBK2, BNDF, GSK3B, GRK3, IMPA1, IMPA2, INPP1, MARCKS, NTRK2 and/or NR1I2 activity.

In one aspect, an animal-based assay of the invention encompasses a method for identifying a test compound for the treatment of mood disorder comprising (a) exposing an animal to a test compound at a concentration and time sufficient to ameliorate an endpoint related to a mood disorder, and (b) determining the level of ADRBK2, BNDF, GSK3B, GRK3, IMPA1, IMPA2, INPP1, MARCKS, NTRK2 and/or NR1I2 activity at a site in said animal. Activity of ADRBK2, BNDF, GSK3B, GRK3, IMPA1, IMPA2, INPP1, MARCKS, NTRK2 and/or NR1I2 can be measured in any suitable cell, tissue or site. Preferably the test compound is a compound capable of or suspected to be capable of ameliorating a symptom of schizophrenia, bipolar disorder or a related disorder. Optionally said test compound is capable or suspected to be capable of modulating ADRBK2, BNDF, GSK3B, GRK3, IMPA1, IMPA2, INPP1, MARCKS, NTRK2 and/or NR1I2 activity. Test compounds capable of modulating ADRBK2, BNDF, GSK3B, GRK3, IMPA1, IMPA2, INPP1, MARCKS, NTRK2 and/or NR1I2 activity may be selected for use in developing therapeutics.

In another aspect, an animal-based assay of the invention encompasses a method for identifying a compound for the treatment of schizophrenia or bipolar disorder comprising (a) exposing an animal to a level of ADRBK2, BNDF, GSK3B, GRK3, IMPA1, IMPA2, INPP1, MARCKS, NTRK2 and/or NR1I2 activity sufficient to cause a schizophrenia-related or bipolar disorder-related symptom or endpoint, and (b) exposing said animal to a test compound. A test compound can then be selected according to its ability to ameliorate said schizophrenia-related or bipolar disorder-related endpoints. Activity of ADRBK2, BNDF, GSK3B, GRK3, IMPA1, IMPA2, INPP1, MARCKS, NTRK2 and/or NR1I2 may be provided by any suitable method, including but not limited to providing a vector containing an ADRBK2, BNDF, GSK3B, GRK3, IMPA1, IMPA2, INPP1, MARCKS, NTRK2 and/or NR1I2 nucleotide sequence, treating said animal with a compound capable of increasing ADRBK2, BNDF, GSK3B, GRK3, IMPA1, IMPA2, INPP1, MARCKS, NTRK2 and/or NR1I2 expression and treating said cell with an ADRBK2, BNDF, GSK3B, GRK3, IMPA1, IMPA2, INPP1, MARCKS, NTRK2 and/or NR1I2 peptide. Preferably, said animal is treated with an ADRBK2, BNDF, GSK3B, GRK3, IMPA1, IMPA2, INPP1, MARCKS, NTRK2 and/or NR1I2 peptide comprising a contiguous span of at least 4 amino acids of sequences corresponding to the SNPs of APPENDIX I. Preferably the test compound is a compound capable of or suspected to be capable of ameliorating a symptom of schizophrenia, bipolar disorder or a related disorder; alternatively, the test compound is suspected of exacerbating a symptom of schizophrenia, bipolar disorder or a related disorder. A test compound capable of ameliorating any detectable symptom or endpoint of a schizophrenia, bipolar disorder or a related disorder may be selected for use in developing medicaments.

Any suitable animal may be used. Preferably, said animal is a primate, a non-human primate, a mammal, or a mouse.

In one embodiment, a mouse is treated with an ADRBK2, BNDF, GSK3B, GRK3, IMPA1, IMPA2, INPP1, MARCKS, NTRK2 and/or NR1I2 peptide, exposed to a test compound, and symptoms indicative of schizophrenia, bipolar disorder or a related disorder are assessed by observing stereotypy. In other embodiments, said symptoms are assessed by performing at least one test from the group consisting of home cage observation, neurological evaluation, stress-induced hypothermia, forced swim, PTZ seizure, locomotor activity, tail suspension, elevated plus maze, novel object recognition, prepulse inhibition, thermal pain, Y-maze, and metabolic chamber tests (Psychoscreen.TM. tests available from Psychogenics Inc.). Other tests are known in Crawley et al, Horm. Behav. 31(3):197-211 (1997); Crawley, Brain Res 835(1):18-26 (1999) for example.

Any suitable test compound may be used with the screening methods of the invention. Examples of compounds that may be screened by the methods of the present invention include small organic or inorganic molecules, nucleic acids, including polynucleotides from random and directed polynucleotide libraries, peptides, including peptides derived from random and directed peptide libraries, soluble peptides, fusion peptides, and phosphopeptides, antibodies including polyclonal, monoclonal, chimeric, humanized, and anti-idiotypic antibodies, and single chain antibodies, FAb, F(ab′).sub.2 and FAb expression library fragments, and epitope-binding fragments thereof. In certain aspects, a compound capable of ameliorating or exacerbating a symptom or endpoint of schizophrenia, bipolar disorder or a related disorder may include, by way of example, antipsychotic drugs in general, neuroleptics, atypical neuroleptics, antidepressants, anti-anxiety drugs, noradrenergic agonists and antagonists, dopaminergic agonists and antagonists, serotonin reuptake inhibitors, benzodiazepines.

In further methods, peptides, drugs, fatty acids, lipoproteins, or small molecules which interact with the ADRBK2, BNDF, GSK3B, GRK3, IMPA1, IMPA2, INPP1, MARCKS, NTRK2 and/or NR1I2 protein, or a fragment comprising a contiguous span of at least 4 amino acids, preferably at least 6, or preferably at least 8 to 10 amino acids, more preferably at least 12, 15, 20, 25, 30, 40, 50, or 100 amino acids of sequences corresponding to the SNPs of APPENDIX I, may be identified using assays such as the following. The molecule to be tested for binding is labeled with a detectable label, such as a fluorescent, radioactive, or enzymatic tag and placed in contact with immobilized ADRBK2, BNDF, GSK3B, GRK3, IMPA1, IMPA2, INPP1, MARCKS, NTRK2 and/or NR1I2 protein, or a fragment thereof under conditions which permit specific binding to occur. After removal of non-specifically bound molecules, bound molecules are detected using appropriate means.

Another object of the present invention comprises methods and kits for the screening of candidate substances that interact with an ADRBK2, BNDF, GSK3B, GRK3, IMPA1, IMPA2, INPP1, MARCKS, NTRK2 and/or NR1I2 polypeptide.

The present invention pertains to methods for screening substances of interest that interact with an ADRBK2, BNDF, GSK3B, GRK3, IMPA1, IMPA2, INPP1, MARCKS, NTRK2 and/or NR1I2 protein or one fragment or variant thereof. By their capacity to bind covalently or non-covalently to an ADRBK2, BNDF, GSK3B, GRK3, IMPA1, IMPA2, INPP1, MARCKS, NTRK2 and/or NR1I2 protein or to a fragment or variant thereof, these substances or molecules may be advantageously used both in vitro and in vivo.

In vitro, said interacting molecules may be used as detection means in order to identify the presence of an ADRBK2, BNDF, GSK3B, GRK3, IMPA1, IMPA2, INPP1, MARCKS, NTRK2 and/or NR1I2 protein in a sample, preferably a biological sample.

A method for the screening of a candidate substance comprises the following steps: a) providing a polypeptide comprising, consisting essentially of, or consisting of an ADRBK2, BNDF, GSK3B, GRK3, IMPA1, IMPA2, INPP1, MARCKS, NTRK2 and/or NR1I2 protein or a fragment comprising a contiguous span of at least 4 amino acids, preferably at least 6 amino acids, more preferably at least 8 to 10 amino acids, more preferably at least 12, 15, 20, 25, 30, 40, 50, or 100 amino acids of sequences corresponding to the SNPs of APPENDIX I; b) obtaining a candidate substance; c) bringing into contact said polypeptide with said candidate substance; and d) detecting the complexes formed between said polypeptide and said candidate substance.

The invention further concerns a kit for the screening of a candidate substance interacting with the ADRBK2, BNDF, GSK3B, GRK3, IMPA1, IMPA2, INPP1, MARCKS, NTRK2 and/or NR1I2 polypeptide, wherein said kit comprises: a) an ADRBK2, BNDF, GSK3B, GRK3, IMPA1, IMPA2, INPP1, MARCKS, NTRK2 and/or NR1I2 protein having an amino acid sequence selected from the group consisting of the amino acid sequences of sequences corresponding to the SNPs of APPENDIX I or a peptide fragment comprising a contiguous span of at least 4 amino acids, preferably at least 6 amino acids, more preferably at least 8 to 10 amino acids, and more preferably at least 12, 15, 20, 25, 30, 40, 50, or 100 amino acids of sequences corresponding to the SNPs of APPENDIX I; and b) optionally means useful to detect the complex formed between the ADRBK2, BNDF, GSK3B, GRK3, IMPA1, IMPA2, INPP1, MARCKS, NTRK2 and/or NR1I2 protein or a peptide fragment or a variant thereof and the candidate substance.

In a preferred embodiment of the kit described above, the detection means comprise monoclonal or polyclonal antibodies directed against the ADRBK2, BNDF, GSK3B, GRK3, IMPA1, IMPA2, INPP1, MARCKS, NTRK2 and/or NR1I2 protein or a peptide fragment or a variant thereof.

Various candidate substances or molecules can be assayed for interaction with an ADRBK2, BNDF, GSK3B, GRK3, IMPA1, IMPA2, INPP1, MARCKS, NTRK2 and/or NR1I2 polypeptide. These substances or molecules include, without being limited to, natural or synthetic organic compounds or molecules of biological origin such as polypeptides. When the candidate substance or molecule comprise a polypeptide, this polypeptide may be the resulting expression product of a phage clone belonging to a phage-based random peptide library, or alternatively the polypeptide may be the resulting expression product of a cDNA library cloned in a vector suitable for performing a two-hybrid screening assay.

The invention also pertains to kits useful for performing the hereinbefore described screening method. Preferably, such kits comprise an ADRBK2, BNDF, GSK3B, GRK3, IMPA1, IMPA2, INPP1, MARCKS, NTRK2 and/or NR1I2 polypeptide or a fragment or a variant thereof, and optionally means useful to detect the complex formed between the ADRBK2, BNDF, GSK3B, GRK3, IMPA1, IMPA2, INPP1, MARCKS, NTRK2 and/or NR1I2 polypeptide or its fragment or variant and the candidate substance. In a preferred embodiment the detection means comprise monoclonal or polyclonal antibodies directed against the corresponding ADRBK2, BNDF, GSK3B, GRK3, IMPA1, IMPA2, INPP1, MARCKS, NTRK2 and/or NR1I2 polypeptide or a fragment or a variant thereof.

A. Candidate Ligands Obtained from Random Peptide Libraries

In a particular embodiment of the screening method, the putative ligand is the expression product of a DNA insert contained in a phage vector (Parmley and Smith, 1988). Specifically, random peptide phages libraries are used. The random DNA inserts encode for peptides of 8 to 20 amino acids in length (Oldenburg K. R. et al., 1992; Valadon P., et al., 1996; Lucas A. H., 1994; Westerink M. A. J., 1995; Felici F. et al., 1991). According to this particular embodiment, the recombinant phages expressing a protein that binds to the immobilized ADRBK2, BNDF, GSK3B, GRK3, IMPA1, IMPA2, INPP1, MARCKS, NTRK2 and/or NR1I2 protein is retained and the complex formed between the ADRBK2, BNDF, GSK3B, GRK3, IMPA1, IMPA2, INPP1, MARCKS, NTRK2 and/or NR1I2 protein and the recombinant phage may be subsequently immunoprecipitated by a polyclonal or a monoclonal antibody directed against the ADRBK2, BNDF, GSK3B, GRK3, IMPA1, IMPA2, INPP1, MARCKS, NTRK2 and/or NR1I2 protein.

Once the ligand library in recombinant phages has been constructed, the phage population is brought into contact with the immobilized ADRBK2, BNDF, GSK3B, GRK3, IMPA1, IMPA2, INPP1, MARCKS, NTRK2 and/or NR1I2 protein. Then the preparation of complexes is washed in order to remove the non-specifically bound recombinant phages. The phages that bind specifically to the ADRBK2, BNDF, GSK3B, GRK3, IMPA1, IMPA2, INPP1, MARCKS, NTRK2 and/or NR1I2 protein are then eluted by a buffer (acid pH) or immunoprecipitated by the monoclonal antibody produced by the hybridoma anti-ADRBK2, BNDF, GSK3B, GRK3, IMPA1, IMPA2, INPP1, MARCKS, NTRK2 and/or NR1I2, and this phage population is subsequently amplified by an over-infection of bacteria (for example E. coli). The selection step may be repeated several times, preferably 24 times, in order to select the more specific recombinant phage clones. The last step comprises characterizing the peptide produced by the selected recombinant phage clones either by expression in infected bacteria and isolation, expressing the phage insert in another host-vector system, or sequencing the insert contained in the selected recombinant phages.

Alternatively, Candidate Ligands Can be obtained by Competition Experiments, Affinity Chromatography, Optical Biosensor Methods, Two-Hybrid Screening Assay, all of such well-known to those of ordinary skill in the art.

Method for Screening Substances Interacting with the Regulatory Sequences of an ADRBK2, BNDF, GSK3B, GRK3, IMPA1, IMPA2, INPP1, MARCKS, NTRK2 and/or NR1I2 Gene

The present invention also concerns a method for screening substances or molecules that are able to interact with the regulatory sequences of the ADRBK2, BNDF, GSK3B, GRK3, IMPA1, IMPA2, INPP1, MARCKS, NTRK2 and/or NR1I2 gene, such as for example promoter or enhancer sequences. Nucleic acids encoding proteins which are able to interact with the regulatory sequences of the ADRBK2, BNDF, GSK3B, GRK3, IMPA1, IMPA2, INPP1, MARCKS, NTRK2 and/or NR1I2 gene, more particularly a nucleotide sequence selected from the group consisting of the polynucleotides of the 5′ and 3′ regulatory region or a fragment or variant thereof, and preferably a variant comprising one of the biallelic markers of the invention, may be identified by using a one-hybrid system, such as that described in the booklet enclosed in the Matchmaker One-Hybrid System kit from Clontech (Catalog Ref. n.sup.o K1603-1), the technical teachings of which are herein incorporated by reference. Briefly, the target nucleotide sequence is cloned upstream of a selectable reporter sequence and the resulting DNA construct is integrated in the yeast genome (Saccharomyces cerevisiae). The yeast cells containing the reporter sequence in their genome are then transformed with a library comprising fusion molecules between cDNAs encoding candidate proteins for binding onto the regulatory sequences of the ADRBK2, BNDF, GSK3B, GRK3, IMPA1, IMPA2, INPP1, MARCKS, NTRK2 and/or NR1I2 gene and sequences encoding the activator domain of a yeast transcription factor such as GAL4. The recombinant yeast cells are plated in a culture broth for selecting cells expressing the reporter sequence. The recombinant yeast cells thus selected contain a fusion protein that is able to bind onto the target regulatory sequence of the ADRBK2, BNDF, GSK3B, GRK3, IMPA1, IMPA2, INPP1, MARCKS, NTRK2 and/or NR1I2 gene. Then, the cDNAs encoding the fusion proteins are sequenced and may be cloned into expression or transcription vectors in vitro. The binding of the encoded polypeptides to the target regulatory sequences of the ADRBK2, BNDF, GSK3B, GRK3, IMPA1, IMPA2, INPP1, MARCKS, NTRK2 and/or NR1I2 gene may be confirmed by techniques familiar to the one skilled in the art, such as gel retardation assays such as described by Fried M. and Crothers D. M. (1981) Nucleic Acids Res.9:6505-6525., Garner M. M. and Revzin A. (1981) Nucleic AcidsRes.9:3047-3060. and Dent D. S. and Latchman D. S. (1993) or DNAse protection assays.

Method for Screening Ligands that Modulate the Expression of the ADRBK2, BNDF, GSK3B, GRK3, IMPA1, IMPA2, INPP1, MARCKS, NTRK2 and/or NR1I2 gene.

Another subject of the present invention is a method for screening molecules that modulate the expression of the ADRBK2, BNDF, GSK3B, GRK3, IMPA1, IMPA2, INPP1, MARCKS, NTRK2 and/or NR1I2 protein. Such a screening method comprises the steps of: a) cultivating a prokaryotic or an eukaryotic cell that has been transfected with a nucleotide sequence encoding the ADRBK2, BNDF, GSK3B, GRK3, IMPA1, IMPA2, INPP1, MARCKS, NTRK2 and/or NR1I2 protein or a variant or a fragment thereof, placed under the control of its own promoter; b) bringing into contact the cultivated cell with a molecule to be tested; c) quantifying the expression of the ADRBK2, BNDF, GSK3B, GRK3, IMPA1, IMPA2, INPP1, MARCKS, NTRK2 and/or NR1I2 protein or a variant or a fragment thereof.

In an embodiment, the nucleotide sequence encoding the ADRBK2, BNDF, GSK3B, GRK3, IMPA1, IMPA2, INPP1, MARCKS, NTRK2 and/or NR1I2 protein or a variant or a fragment thereof comprises an allele of at least one ADRBK2, BNDF, GSK3B, GRK3, IMPA1, IMPA2, INPP1, MARCKS, NTRK2 and/or NR1I2 related biallelic marker.

Using DNA recombination techniques well known by the one of ordinary skill in the art, the ADRBK2, BNDF, GSK3B, GRK3, IMPA1, IMPA2, INPP1, MARCKS, NTRK2 and/or NR1I2 protein encoding DNA sequence is inserted into an expression vector, downstream from its promoter sequence. As an illustrative example, the promoter sequence of the ADRBK2, BNDF, GSK3B, GRK3, IMPA1, IMPA2, INPP1, MARCKS, NTRK2 and/or NR1I2 gene is contained in the nucleic acid of the 5′ regulatory region.

The quantification of the expression of the ADRBK2, BNDF, GSK3B, GRK3, IMPA1, IMPA2, INPP1, MARCKS, NTRK2 and/or NR1I2 protein may be realized either at the mRNA level or at the protein level. In the latter case, polyclonal or monoclonal antibodies may be used to quantify the amounts of the ADRBK2, BNDF, GSK3B, GRK3, IMPA1, IMPA2, INPP1, MARCKS, NTRK2 and/or NR1I2 protein that have been produced, for example in an ELISA or a RIA assay.

In a preferred embodiment, the quantification of the ADRBK2, BNDF, GSK3B, GRK3, IMPA1, IMPA2, INPP1, MARCKS, NTRK2 and/or NR1I2 mRNA is realized by a quantitative PCR amplification of the cDNA obtained by a reverse transcription of the total mRNA of the cultivated ADRBK2, BNDF, GSK3B, GRK3, IMPA1, IMPA2, INPP1, MARCKS, NTRK2 and/or NR1I2-transfected host cell, using a pair of primers specific for ADRBK2, BNDF, GSK3B, GRK3, IMPA1, IMPA2, INPP1, MARCKS, NTRK2 and/or NR1I2.

The present invention also concerns a method for screening substances or molecules that are able to increase, or in contrast to decrease, the level of expression of the ADRBK2, BNDF, GSK3B, GRK3, IMPA1, IMPA2, INPP1, MARCKS, NTRK2 and/or NR1I2 gene. Such a method may allow the one skilled in the art to select substances exerting a regulating effect on the expression level of the ADRBK2, BNDF, GSK3B, GRK3, IMPA1, IMPA2, INPP1, MARCKS, NTRK2 and/or NR1I2 gene and which may be useful as active ingredients included in pharmaceutical compositions for treating patients suffering from diseases.

Thus, is also part of the present invention a method for screening of a candidate substance or molecule that modulated the expression of the ADRBK2, BNDF, GSK3B, GRK3, IMPA1, IMPA2, INPP1, MARCKS, NTRK2 and/or NR1I2 gene, this method comprises the following steps: a) providing a recombinant cell host containing a nucleic acid, wherein said nucleic acid comprises a nucleotide sequence of the 5′ regulatory region or a biologically active fragment or variant thereof located upstream a polynucleotide encoding a detectable protein; b) obtaining a candidate substance; and c) determining the ability of the candidate substance to modulate the expression levels of the polynucleotide encoding the detectable protein.

Among the preferred polynucleotides encoding a detectable protein, there may be cited polynucleotides encoding beta galactosidase, green fluorescent protein (GFP) and chloramphenicol acetyl transferase (CAT).

The invention also pertains to kits useful for performing the herein described screening method. Preferably, such kits comprise a recombinant vector that allows the expression of a nucleotide sequence of the 5′ regulatory region or a biologically active fragment or variant thereof located upstream and operably linked to a polynucleotide encoding a detectable protein or the ADRBK2, BNDF, GSK3B, GRK3, IMPA1, IMPA2, INPP1, MARCKS, NTRK2 and/or NR1I2 protein or a fragment or a variant thereof.

In another embodiment of a method for the screening of a candidate substance or molecule that modulates the expression of the ADRBK2, BNDF, GSK3B, GRK3, IMPA1, IMPA2, INPP1, MARCKS, NTRK2 and/or NR1I2 gene, wherein said method comprises the following steps: a) providing a recombinant host cell containing a nucleic acid, wherein said nucleic acid comprises a 5′UTR sequence of an ADRBK2, BNDF, GSK3B, GRK3, IMPA1, IMPA2, INPP1, MARCKS, NTRK2 and/or NR1I2 cDNA, or one of its biologically active fragments or variants, the 5′UTR sequence or its biologically active fragment or variant being operably linked to a polynucleotide encoding a detectable protein; b) obtaining a candidate substance; and c) determining the ability of the candidate substance to modulate the expression levels of the polynucleotide encoding the detectable protein.

In a specific embodiment of the above screening method, the nucleic acid that comprises a nucleotide sequence selected from the group consisting of the 5′UTR sequence of an ADRBK2, BNDF, GSK3B, GRK3, IMPA1, IMPA2, INPP1, MARCKS, NTRK2 and/or NR1I2 cDNA, or one of its biologically active fragments or variants, includes a promoter sequence which is endogenous with respect to the ADRBK2, BNDF, GSK3B, GRK3, IMPA1, IMPA2, INPP1, MARCKS, NTRK2 and/or NR1I2 5′UTR sequence.

In another specific embodiment of the above screening method, the nucleic acid that comprises a nucleotide sequence selected from the group consisting of the 5′UTR sequence of an ADRBK2, BNDF, GSK3B, GRK3, IMPA1, IMPA2, INPP1, MARCKS, NTRK2 and/or NR1I2 cDNA or one of its biologically active fragments or variants, includes a promoter sequence which is exogenous with respect to the ADRBK2, BNDF, GSK3B, GRK3, IMPA1, IMPA2, INPP1, MARCKS, NTRK2 and/or NR1I2 5′UTR sequence defined therein.

In a further preferred embodiment, the nucleic acid comprising the 5′-UTR sequence of an ADRBK2, BNDF, GSK3B, GRK3, IMPA1, IMPA2, INPP1, MARCKS, NTRK2 and/or NR1I2 cDNA or the biologically active fragments thereof includes an ADRBK2, BNDF, GSK3B, GRK3, IMPA1, IMPA2, INPP1, MARCKS, NTRK2 and/or NR1I2-related biallelic marker.

The invention further comprises a kit for the screening of a candidate substance modulating the expression of the ADRBK2, BNDF, GSK3B, GRK3, IMPA1, IMPA2, INPP1, MARCKS, NTRK2 and/or NR1I2 gene, wherein said kit comprises a recombinant vector that comprises a nucleic acid including a 5′UTR sequence of the ADRBK2, BNDF, GSK3B, GRK3, IMPA1, IMPA2, INPP1, MARCKS, NTRK2 and/or NR1I2 cDNA of the nucleotides detailed in APPENDIX I, or one of their biologically active fragments or variants, the 5′UTR sequence or its biologically active fragment or variant being operably linked to a polynucleotide encoding a detectable protein.

Expression levels and patterns of ADRBK2, BNDF, GSK3B, GRK3, IMPA1, IMPA2, INPP1, MARCKS, NTRK2 and/or NR1I2 may be analyzed by solution hybridization with long probes as described in International Patent Application No. WO 97/05277, the entire contents of which are incorporated herein by reference. Briefly, the ADRBK2, BNDF, GSK3B, GRK3, IMPA1, IMPA2, INPP1, MARCKS, NTRK2 and/or NR1I2 cDNA or ADRBK2, BNDF, GSK3B, GRK3, IMPA1, IMPA2, INPP1, MARCKS, NTRK2 and/or NR1I2 genomic DNA described above, or fragments thereof, is inserted at a cloning site immediately downstream of a bacteriophage (T3, T7 or SP6) RNA polymerase promoter to produce antisense RNA. Preferably, ADRBK2, BNDF, GSK3B, GRK3, IMPA1, IMPA2, INPP1, MARCKS, NTRK2 and/or NR1I2 insert comprises at least 100 or more consecutive nucleotides of the genomic DNA sequence or the cDNA sequences. The plasmid is linearized and transcribed in the presence of ribonucleotides comprising modified ribonucleotides (i.e. biotin-UTP and DIG-UTP). An excess of this doubly labeled RNA is hybridized in solution with mRNA isolated from cells or tissues of interest. The hybridization is performed under standard stringent conditions (40-50.degree. C. for 16 hours in an 80% formamide, 0.4 M NaCl buffer, pH 7-8). The unhybridized probe is removed by digestion with ribonucleases specific for single-stranded RNA (i.e. RNases CL3, T1, Phy M, U2 or A). The presence of the biotin-UTP modification enables capture of the hybrid on a microtitration plate coated with streptavidin. The presence of the DIG modification enables the hybrid to be detected and quantified by ELISA using an anti-DIG antibody coupled to alkaline phosphatase.

Quantitative analysis of ADRBK2, BNDF, GSK3B, GRK3, IMPA1, IMPA2, INPP1, MARCKS, NTRK2 and/or NR1I2 gene expression may also be performed using arrays. As used herein, the term array means a one dimensional, two dimensional, or multidimensional arrangement of a plurality of nucleic acids of sufficient length to permit specific detection of expression of mRNAs capable of hybridizing thereto. For example, the arrays may contain a plurality of nucleic acids derived from genes whose expression levels are to be assessed. The arrays may include ADRBK2, BNDF, GSK3B, GRK3, IMPA1, IMPA2, INPP1, MARCKS, NTRK2 and/or NR1I2 genomic DNA, the ADRBK2, BNDF, GSK3B, GRK3, IMPA1, IMPA2, INPP1, MARCKS, NTRK2 and/or NR1I2 cDNA sequences or the sequences complementary thereto or fragments thereof, particularly those comprising at least one of the biallelic markers according the present invention. Preferably, the fragments are at least 15 nucleotides in length. In other embodiments, the fragments are at least 25 nucleotides in length. In some embodiments, the fragments are at least 50 nucleotides in length. More preferably, the fragments are at least 100 nucleotides in length. In another preferred embodiment, the fragments are more than 100 nucleotides in length. In some embodiments the fragments may be more than 500 nucleotides in length. Quantitative analysis may be performed by the method described by Schena et al. (1995)Science. 270:467-470 Lockhart et al. (1996) Nature Biotechnology 14:1675-1680 and Sosnowski R. G. et al. (1997) Proc. Natl. Acad.Sci. U.S.A. 94:1119-1123 or Pietu et al. (1996) Genome Research.6:492-503.

Effective Dosage.

Pharmaceutical compositions suitable for use in the present invention include compositions wherein the active ingredients are contained in an effective amount to achieve their intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

For any compound used in the method of the invention, the therapeutically effective dose can be estimated initially from cell culture assays, and a dose can be formulated in animal models. Such information can be used to more accurately determine useful doses in humans.

A therapeutically effective dose refers to that amount of the compound that results in amelioration of symptoms in a patient. Toxicity and therapeutic efficacy of such compounds can be determined by standard pharmaceutical procedures in cell cultures or experimental animals, e.g., for determining the LD50, (the dose lethal to 50% of the test population) and the ED50 (the dose therapeutically effective in 50% of the population). The dose ratio between toxic and therapeutic effects is the therapeutic index and it can be expressed as the ratio between LD50 and ED50. Compounds which exhibit high therapeutic indices are preferred.

The data obtained from these cell culture assays and animal studies can be used in formulating a range of dosage for use in human. The dosage of such compounds lies preferably within a range of circulating concentrations that include the ED50, with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, in “The Pharmacological Basis of Therapeutics”, Ch. 1).

Therapeutic treatment of Mood disorders:

Suppressing the effects of the mutations through antisense or short interfering (siRNA) technology provides an effective therapy for Mood disorders. Much is known about ‘antisense’ or siRNA therapies targeting messenger RNA (mRNA) or nuclear DNA. Hlen et al., Biochem. Biophys. Acta 1049:99-125 (1990). The diagnostic test of the present invention is useful for determining which of the specific Mood disorders mutations exist in a particular Mood disorders patient; this allows for “custom” treatment of the patient with antisense or siRNA oligonucleotides only for the detected mutations. This patient-specific antisense therapy is also novel, and minimizes the exposure of the patient to any unnecessary antisense or siRNA therapeutic treatment. As used herein, an “antisense” oligonucleotide is one that base pairs with single stranded DNA or RNA by Watson-Crick base pairing and with duplex target DNA via Hoogsteen hydrogen bonds.

RNA interference refers to the process of sequence-specific post-transcriptional gene silencing in animals mediated by short interfering RNAs (siRNAs). The process of post-transcriptional gene silencing is an evolutionarily conserved cellular defense mechanism believed to prevent the expression of foreign genes. Such protection from foreign gene expression may have evolved in response to the production of double-stranded RNAs (dsRNAs) derived from viral infection, or from the random integration of transposon elements into the host genome. The presence of dsRNA in cells triggers the RNAi response through a mechanism that has yet to be fully characterized. The presence of long dsRNAs in cells stimulates the activity of a ribonuclease III enzyme referred to as dicer.

Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also uses an endonuclease complex, commonly referred to as an RNA-induced silencing complex (RISC). RISC mediates cleavage of single-stranded RNA having sequence complementarity to the antisense strand associated with the complex. RNA interference (RNAi) has been harnessed in laboratory cell culture systems and widely applied to identify the function of genes and their respective proteins. Moreover, RNAi holds promise for the development of a brand new class of drugs, capable of turning off disease-causing genes. These drugs could have specificity and potential applications in a number of therapeutic indications. For more detail see U.S. Pat. No. 5,854,038 entitled ‘Localization Of Therapeutic Agent In A Cell In Vitro’.

Another preferred methodology uses DNA directed RNA interference (ddRNAi). ddRNAi relies on RNA polymerase III (Pol III) promoters (e.g. U6 or H1) for the expression of siRNA target sequences that have been transfected in mammalian cells.

Pol III directs the synthesis of small RNA transcripts whose 3′ ends are defined by termination within a stretch of 4-5 thymidines. These characteristics allow for the use of DNA templates to synthesize, in vivo, small RNA duplexes that are structurally equivalent to active siRNAs synthesized in vitro.

siRNA/RISC duplexes form in the cell and lead to the degradation of the target mRNA. siRNA target sequences then can be introduced into the cell by a ddRNAi expression cassette or by being cloned in a siRNA expression vector. For more detail see Gou, D. et al. (2003) Gene Silencing in mammalian cells by PCR-based short hairpin RNA FEBS 548, 113-118.

The destructive effect of the Mood disorders mutations in ADRBK2, BNDF, GSK3B, GRK3, IMPA1, IMPA2, INPP1, MARCKS, NTRK2 and/or NR1I2 gene(s) is preferably reduced or eliminated using antisense or siRNA oligonucleotide agents. Such antisense agents target DNA, by triplex formation with double-stranded DNA, by duplex formation with single-stranded DNA during transcription, or both. In a preferred embodiment, antisense agents target messenger RNA coding for the mutated ADRBK2, BNDF, GSK3B, GRK3, IMPA1, IMPA2, INPP1, MARCKS, NTRK2 and/or NR1I2 gene(s). Since the sequences of both the DNA and the mRNA are the same, it is not necessary to determine accurately the precise target to account for the desired effect. Procedures for inhibiting gene expression in cell culture and in vivo can be found, for example, in C. F. Bennett, et al. J. Liposome Res., 3:85 (1993) and C. Wahlestedt, et al. Nature, 363:260 (1993).

Antisense oligonucleotide therapeutic agents demonstrate a high degree of pharmaceutical specificity. This allows the combination of two or more antisense therapeutics at the same time, without increased cytotoxic effects. Thus, when a patient is diagnosed as having two or more Mood disorders mutations in ADRBK2, BNDF, GSK3B, GRK3, IMPA1, IMPA2, INPP1, MARCKS, NTRK2 and/or NR1I2 gene(s), the therapy is preferably tailored to treat the multiple mutations simultaneously. When combined with the present diagnostic test, this approach to “patient-specific therapy” results in treatment restricted to the specific mutations detected in a patient. This patient-specific therapy circumvents the need for ‘broad spectrum’ antisense treatment using all possible mutations. The end result is less costly treatment, with less chance for toxic side effects.

One method to inhibit the synthesis of proteins is through the use of antisense or triplex oligonucleotides, analogues or expression constructs. These methods entail introducing into the cell a nucleic acid sufficiently complementary in sequence so as to specifically hybridize to the target gene or to mRNA. In the event that the gene is targeted, these methods can be extremely efficient since only a few copies per cell are required to achieve complete inhibition. Antisense methodology inhibits the normal processing, translation or half-life of the target message. Such methods are well known to one skilled in the art.

Antisense and triplex methods generally involve the treatment of cells or tissues with a relatively short oligonucleotide, although longer sequences can be used to achieve inhibition. The oligonucleotide can be either deoxyribo- or ribonucleic acid and must be of sufficient length to form a stable duplex or triplex with the target RNA or DNA at physiological temperatures and salt concentrations. It should also be sufficiently complementary or sequence specific to specifically hybridize to the target nucleic acid. Oligonucleotide lengths sufficient to achieve this specificity are preferably about 10 to 60 nucleotides long, more preferably about 10 to 20 nucleotides long. However, hybridization specificity is not only influenced by length and physiological conditions but may also be influenced by such factors as GC content and the primary sequence of the oligonucleotide. Such principles are well known in the art and can be routinely determined by one who is skilled in the art.

As an example, many of the oligonucleotide sequences used in connection with probes can also be used as antisense agents, directed to either the DNA or resultant messenger RNA.

A great range of antisense sequences can be designed for a given mutation. Oligonucleotide sequences can be easily designed by one of ordinary skill in the art to function as RNA and DNA antisense sequences for the mutant genes ADRBK2, BNDF, GSK3B, GRK3, IMPA1, IMPA2, INPP1, MARCKS, NTRK2 and/or NR1I2 gene(s).

As can be seen, permutations can be generated for a selected mutant antigene by truncating the 5′ end, truncating the 3′ end, extending the 5′ end, or extending the 3′ end. Both light chain and heavy chain mtDNA can be targeted. Other variations such as truncating the 5′ end and truncating the 3′ end, extending the 5′ end and extending the 3′ end, and truncating the 5′ end and extending the 3′ end, extending the 5′ end and truncating the 3′ end, and so forth are possible.

The composition of the antisense or triplex oligonucleotides can also influence the efficiency of inhibition. For example, it is preferable to use oligonucleotides that are resistant to degradation by the action of endogenous nucleases. Nuclease resistance will confer a longer in vivo half-life to the oligonucleotide thus increasing its efficacy and reducing the required dose. Greater efficacy may also be obtained by modifying the oligonucleotide so that it is more permeable to cell membranes. Such modifications are well known in the art and include the alteration of the negatively charged phosphate backbone bases, or modification of the sequences at the 5′ or 3′ terminus with agents such as intercalators and crosslinking molecules. Specific examples of such modifications include oligonucleotide analogs that contain methylphosphonate (Miller, P. S., Biotechnology, 2:358-362 (1991)), phosphorothioate (Stein, Science 261:1004-1011 (1993)) and phosphorodithioate linkages (Brill, W. K-D., J. Am. Chem. Soc., 111:2322 (1989)). Other types of linkages and modifications exist as well, such as a polyamide backbone in peptide nucleic acids (Nielson et al., Science 254:1497 (1991)), formacetal (Matteucci, M., Tetrahedron Lett. 31:2385-2388 (1990)) carbamate and morpholine linkages as well as others known to those skilled in the art. In addition to the specificity afforded by the antisense agents, the target RNA or genes can be irreversibly modified by incorporating reactive functional groups in these molecules which covalently link the target sequences e.g. by alkylation.

Recombinant methods known in the art can also be used to achieve the antisense or triplex inhibition of a target nucleic acid. For example, vectors containing antisense nucleic acids can be employed to express protein or antisense message to reduce the expression of the target nucleic acid and therefore its activity. Such vectors are known or can be constructed by those skilled in the art and should contain all expression elements necessary to achieve the desired transcription of the antisense or triplex sequences. Other beneficial characteristics can also be contained within the vectors such as mechanisms for recovery of the nucleic acids in a different form. Phagemids are a specific example of such beneficial vectors because they can be used either as plasmids or as bacteriophage vectors. Examples of other vectors include viruses, such as bacteriophages, baculoviruses and retroviruses, cosmids, plasmids, liposomes and other recombination vectors. The vectors can also contain elements for use in either procaryotic or eukaryotic host systems. One of ordinary skill in the art will know which host systems are compatible with a particular vector.

The vectors can be introduced into cells or tissues by any one of a variety of known methods within the art. Such methods are described for example in Sambrook et al., Molecular Cloning: A Laboratory Manual, Cold Spring Harbor Laboratory, New York (1992), which is hereby incorporated by reference, and in Ausubel et al., Current Protocols in Molecular Biology, John Wiley and Sons, Baltimore, Md. (1989), which is also hereby incorporated by reference. The methods include, for example, stable or transient transfection, lipofection, electroporation and infection with recombinant viral vectors. Introduction of nucleic acids by infection offers several advantages over the other listed methods which includes their use in both in vitro and in vivo settings. Higher efficiency can also be obtained due to their infectious nature. Moreover, viruses are very specialized and typically infect and propagate in specific cell types. Thus, their natural specificity can be used to target the antisense vectors to specific cell types in vivo or within a tissue or mixed culture of cells. Viral vectors can also be modified with specific receptors or ligands to alter target specificity through receptor mediated events.

A specific example of a viral vector for introducing and expressing antisense nucleic acids is the adenovirus derived vector Adenop53TX. This vector expresses a herpes virus thymidine kinase (TX) gene for either positive or negative selection and an expression cassette for desired recombinant sequences such as antisense sequences. This vector can be used to infect cells including most cancers of epithelial origin, glial cells and other cell types. This vector as well as others that exhibit similar desired functions can be used to treat a mixed population of cells to selectively express the antisense sequence of interest. A mixed population of cells can include, for example, in vitro or ex vivo culture of cells, a tissue or a human subject.

Additional features may be added to the vector to ensure its safety and/or enhance its therapeutic efficacy. Such features include, for example, markers that can be used to negatively select against cells infected with the recombinant virus. An example of such a negative selection marker is the TK gene described above that confers sensitivity to the antibiotic gancyclovir. Negative selection is therefore a means by which infection can be controlled because it provides inducible suicide through the addition of antibiotics. Such protection ensures that if, for example, mutations arise that produce mutant forms of the viral vector or antisense sequence, cellular transformation will not occur. Moreover, features that limit expression to particular cell types can also be included. Such features include, for example, promoter and expression elements that are specific for the desired cell type.

Methodology of Marker Selection, Analysis, and Classification

Non-linear techniques for data analysis and information extraction are important for identifying complex interactions between markers that contribute to overall presentation of the clinical outcome. However, due to the many features involved in association studies such as the one proposed, the construction of these in-silico predictors is a complex process. Often one must consider more markers to test than samples, missing values, poor generalization of results, selection of free parameters in predictor models, confidence in finding a sub-optimal solution and others. Thus, the process for building a predictor is as important as designing the protocol for the association studies. Errors at each step can propagate downstream, affecting the generalizability of the final result.

We now provide an overview of our process of model development, describing the five main steps and some techniques that the instant invention will use to build an optimal biomarker panel of response for each clinical outcome. One of ordinary skill in the art will know that it is best to use a ‘toolbox’ approach to the various steps, trying several different algorithms at each step, and even combining several as in Step Five. Since one does not know a priori the distribution of the true solution space, trying several methods allows a thorough search of the solution space of the observed data in order to find the most optimal solutions (i.e. those best able to generalize to unseen data). One also can give more confidence to predictions if several independent techniques converge to a similar solution.

Data Pre-processing

After assaying the patients for various markers, it is necessary to perform some basic data ‘inspection’, such as identification of outliers, before starting a program of outcome prediction. Another task is performing data dimensional shifting in the case of discrete data sets such as SNP analysis. For instance, one can describe a three-state SNP vector either three-dimensionally (1,0,0);(0,1,0);(0,0,1) or two-dimensionally (0,0);(1,0);(0,1). For some algorithms, the latter description may have a direct effect on computational cost and classifier accuracy: one can, in effect, collapse several values to a single parameter. The advantage of single parameter is that one can reduce dimensionality with little or no effect on the selection of the optimal feature set. Following pre-processing, one can then perform univariate and multivariate statistical modeling to identify strongly correlative outcome variables and determine a baseline outcome analysis.

Missing Value Estimation

While the call rate and accuracy of high throughput methods are improving, genotype and proteomic data sets usually contain missing values. Missing values arise from missed genotype calls or from the combination of data collected under different protocols. If subsequent analysis requires complete data sets, repeating the experiment can be expensive and removing rows or columns containing missing values in the data set may be wasteful.

Missing values can be replaced with the most likely genotype based on frequency estimates for an individual marker. This row counting method may be sufficient when few markers are genotyped, but it is not optimal for genome wide scans since it does not consider correlation in the data. Other statistical approaches to estimating missing values apply genetic models of inheritance. In large-scale association studies of unrelated participants, lineage information is unavailable. For the dataset gathered in the instant invention, we will apply techniques that do not use complex models and take into account the possibly discrete nature of marker data when models are used. These methods fall into two categories: KNN-based and Bayesian-based methods.

KNN estimates the value of the missing data as the most prevalent genotype among the K Nearest Neighbors. For a data set consisting of M patients and N SNPs, the data is stored in an M by N matrix. For each row with a missing value in a single column, the algorithm locates the K nearest neighbors in the N-1 dimensional subspace. The K nearest neighbors then votes to replace the missing value under majority rule. Ties are broken by random draw. If there are n missing values present in a row, we find the nearest neighbors in the N-n subspace.

The only other consideration is what distance function to use to determine the K nearest neighbors. Typically, the Euclidean distance is well suited for continuous data and the Hamming distance for nominal data. The Hamming distance counts the number of different marker genotypes in the N-n subspace and does not impose an artificial ordinality as does the Euclidean distance. There are other options such as the Manhattan distance, the correlation coefficient, and others that may be used depending on the data set distribution.

In contrast, Bayesian imputation uses probabilities instead of distances to infer missing values. The objective is to draw an inference about a missing value for a matrix entry in the data set from the posterior probability of the missing value given the observed data, □(Yₘᵢₛₛ|Y_{obs}), where Y_{obs} is the set of N-n observed marker values and Yₘᵢₛₛ is the missing value. By Bayes's theorem, □(Yₘᵢₛₛ|Y_{obs}) can be expressed as follows:

$$π ( {Y}_{miss} \vert {Y}_{obs} ) = \frac{π ( {Y}_{obs} \vert {Y}_{mis} ) π ( {Y}_{miss} )}{{\sum }_{k = 1}^{m} π ( {Y}_{obs} \vert {Y}_{mis} ) π ( {Y}_{miss} )} \qquad ( 1 )$$

where π(Yₘᵢₛₛ) is the probability that a randomly selected missing entry will have the value Yₘᵢₛₛ, π(Y_{obs}|Yₘᵢₛₛ) is the probability of observing the N-n genotypes given Yₘᵢₛₛ, and the sum is over the m possible values for Yₘᵢₛₛ.

The likelihood model assumes that the probabilities π(Y_{obs}|Yₘᵢₛₛ) can be expressed as functions of unknown parameters of the genotypes Yₘᵢₛₛ:

$$π ( {Y}_{obs = g} \vert {Y}_{miss = k} ) = π ( {y}_{g1} \vert {θ}_{1 k} ) π ( {y}_{g2} \vert {θ}_{2 k} ) K π ( {y}_{gn} \vert {θ}_{nk} ) \\ = {\prod }_{i = 1}^{N - n} π ( {y}_{gi} \vert {θ}_{ik} ) \qquad ( 2 )$$

where θᵢₖ are unknown parameters of Yₘᵢₛₛ for the N-n observed markers, y_{gi} is the i th marker in the set of Y_{obs} markers, and θ(y_{gi}|θᵢₖ) is the probability of observing y_{gi} given the parameter θᵢₖ of the marker value Yₘᵢₛₛ for variable i. The model is based on the assumption that the probability of observing y_{gi} is independent of the probability of observing y_{gi} for each marker value Yₘᵢₛₛ with i≠j.

Missing values are imputed as follows. For each marker for which there is a missing value, the probabilities θ(y_{gi}|θᵢₖ) are estimated based on the observed markers. Using Bayes' theorem, the posterior probability θ(Yₘᵢₛₛ|Y_{obs}) is calculated. We then sample Yₘᵢₛₛ from the posterior. This approach treats the missing value problem as a supervised learning problem in which posterior probability is learned from the pattern of observed markers.

Feature Selection

Following missing value replacement, the third step in the predictive panel building process is to perform feature selection on the dataset; this is perhaps the most important step in the predictor development process. Feature selection serves two purposes: (1) to reduce dimensionality of the data and improve classification accuracy, and (2) to identify biomarkers that are relevant to the cause and consequences of disease and drug response.

A feature selection algorithm (FSA) is a computational solution that given a set of candidate features selects a subset of relevant features with the best commitment among its size and the value of its evaluation measure. However, the relevance of a feature, as seen from the classification perspective, may have several definitions depending on the objective desired. An irrelevant feature is not useful for classification, but not all relevant features are necessarily useful for classification.

Another problem from which many classification methods suffer is the curse of dimensionality. That is, as the number of features in a classification task increases, the time requirements for an algorithm grow dramatically, sometimes exponentially. Therefore, when the set of features in the data is sufficiently large, many classification algorithms are simply intractable. This problem is further exacerbated by the fact that many features in a learning task may either be irrelevant or redundant to other features with respect to predicting the class of an instance. In this context, such features serve no purpose except to increase classification time.

FSAs can be divided into two categories based on whether or not feature selection is done independently of the learning algorithm used to construct the classifier. If the feature selection is independent of the learning algorithm, the technique is said to follow a filter approach. Otherwise, it is said to follow a wrapper approach. While the filter approach is generally computationally more efficient than the wrapper approach, a drawback is that an optimal selection of features may not be independent of the inductive and representational biases of the learning algorithm to be used to construct the classifier.

SFS/SBS

A sequential forward search (SFS), or backward (SBS), is a process that uses an iterative technique for feature selection. In this wrapper technique, one feature at a time is added (SFS) or deleted (SBS) to a set of pre-selected features, and iterated according to a performance metric until the ‘optimal’ set of features are obtained. For example, SFS is a technique that starts with all possible two-variable input combinations from the entire data set and then builds, one variable at a time, until an optimally performing combination of variables is identified. For instance, with 9 input variables labeled 1-9 (each with a binary descriptor), the two-variable combinations would comprise 1|2, 1|3, 1|4, 1|5, 1|6, 1|7, 1|8, 1|9, 2|3, 2|4, 2|5, 2|6 . . . 8|9. These input combinations are each used in training a classifier using the collected data. The combinations that perform the best (evaluated using leave-one-out cross validation; top 10%, for example) are selected for continued addition of variables. Let us say that 2|3 is selected as one of the top performers, it would then be coupled to each of the other variables, not including those variables that are already included in the combination. This would result in 2|3|1, 2|3|4, 2|3|5, 2|3|6, 2|3|7, 2|3|8 and 2|3|9. This coupling is performed for all of the top two-variable performers. The resultant three-variable input combinations are used to train a classifier using the collected data and then evaluated. The top performers are selected and then coupled again with all variables in the group, again used to train a classifier. This is repeated until a maximal predictive accuracy is achieved. In our experience we have noticed a well defined ‘hump’ at the point where the addition of variables into the system results begins to contribute to degradation of system performance.

SBS starts with the full set of features and eliminates those based upon a performance metric. Although in theory, going backward from the full set of features may capture interacting features more easily, the drawback of this method is that it is computationally expensive.

An example of this is described in U.S. patent application Ser. No. 09/611,220, incorporated in entirety with all figures by reference, which uses a variation on the SBS technique. In this method, a Genetic Algorithm (please see section on classifiers) is used in combination with a neural network to create and select child features based upon a fitness ranking that takes into effect multiple performance measures such as sensitivity and specificity. Only top-ranked child features are used in iterating the algorithm forward.

SFFS

The SFS algorithm suffers from a so-called nesting effect. That is, once a feature has been chosen, there is no way for it to be discarded. To overcome this problem, the sequential forward floating algorithm (SFFS) was proposed. SFFS is an exponential cost algorithm that operates in a sequential manner. In each selection step SFFS performs a forward step followed by a variable number of backward ones. In essence, a feature is first unconditionally added and then features are removed as long as the generated subsets are the best among their respective size. The algorithm is so-called because it has the characteristic of floating around a potentially good solution of the specified size.

E-RFE

The Recursive Feature Elimination (RFE) is a well-known feature selection method for support vector machines (SVMs, please see section on classifiers). As a brief overview, a SVM realizes a classification function

$$f ( x ) = {\sum }_{i = 1}^{N} {α}_{i} {γ}_{i} K ( {x}_{i} , x ) + b ,$$

where the coefficients α=(αᵢ)and b are obtained by training over a set of examples S={(xᵢ, yᵢ} I I=1, . . . ,N, xᵢ ε Rⁿ, yᵢ ε {−1, 1} and) K(xᵢx) is the chosen kernel. In the linear case, the SVM expansion defines the hyperplane
f(x)=<w,x>+b, with

$$w = {\sum }_{i = 1}^{N} {α}_{i} {γ}_{i} {x}_{i} .$$

The idea is to define the importance of a feature for a SVM in terms of its contribution to a cost function J (α). At each step of the RFE procedure, a SVM is trained on the given data set, J is computed and the feature less contributing to J is discarded. In the case of linear SVM, the variation due to the elimination of the i-th feature is δJ(i)=wᵢ²; in the non linear case, δJ(i)=½α^{t}Z [symbol] _{i,} ½α^{t}Z(−i) [symbol] where Zᵢⱼ=yᵢyⱼ K (xᵢ, xⱼ). The heavy computational cost of RFE is a function of the number of variables, as another SVM must be trained each time a variable is removed. In the standard RFE algorithm we would eliminate just one of the many features corresponding to a minimum weight, while it would be convenient to remove all of them at once. We will go further in the instant invention by developing an ad hoc strategy for an elimination process based on the structure of the weight distribution. This strategy was first described by Furlanello (24). We introduce an entropy function H as a measure of the weight distribution. To compute the entropy, we split the range of the weights, normalized in the unit interval, into nᵢₙₜ intervals (with nᵢₙₜ=\sqrt{#R}), and we compute for each interval the relative frequencies

$${p}_{i} = \frac{\# δ J ( i )}{\# R} , i = 1 , … , {n}_{int}$$

Entropy is then defined as the following function:

$$H = - {\sum }_{i = 1}^{{n}_{int}} {p}_{i} {log}_{2} {p}_{i}$$

The following inequality immediately descends from the definition of entropy: 0≦H≦log₂nᵢₙₜ, the two bounds corresponding to the situations:
H=0; or all the weights lie in one interval;
H=log₂nᵢₙₜ; or all the intervals contain the same number of weights.
   The new entropy-based RFE (E-RFE) algorithm eliminates chunks of features at every loop, with two different procedures applied for lower or higher values of H. The distinction is needed to remove many features that have a similar (low) weight while preserving the residual distribution structure, and also allowing for differences between classification problems. E-RFE has been shown to speed up RFE by a factor of 100.

URG

One filter method especially suited for ordinal data has been developed recently by the authors of the instant invention, and offers clearly interpretable results on such data. The feature selection aspect, tentatively named URG, or Universal Regressor Gauge, is a general method for scoring and ranking the predictive sensitivity of input variables by fitting the gauge, or the scaling, on each of the input variables subject to both predictive accuracy of a nonparametric regression, and a penalty on the LI norm of the vector of scaling parameters. The result is a sampled-gradient local minimum solution that does not require assumptions of linearity or exhaustive power-set sampling of subsets of variables. The approach penalizes the gauge θ, or the set of scaling parameters (θ₁, θ₂, . . . θₙ), applied to each of the input variables. The authors of the instant invention generalized this method to potentially nonlinear, nonparametric models of arbitrary complexity using a kernel-based nonparametric regressor. The penalty on the gauge is regularized by a coefficient λ that is scanned across a range of values to put progressively more downward pressure on the scaling parameters, forcing the scale (and the resulting significance in distance-based regression) downward first on those variables that can be most easily eliminated without sacrificing accuracy. Because this process is analog in the state-space of the gauge, nonlinear interactions between subsets can be investigated in a continuous manner, even if the variables themselves are discrete-valued.

Other FSAs complentated, but not limited to, to be used in the instant invention include HITON Markov Blankets and Bayesian filters.

Classification

The fourth step in the predictor-building process is classification. In the supervised learning task, one is given a training set of labeled fixed-length feature vectors, from which to induce a classification model. This model, in turn, is used to predict the class label for a set of previously unseen instances. Thus, in building a classification model, the information about the class that is inherent in the features is of utmost importance. The dataset that the classifier is trained upon is broken up generally into three different sets: Training, Testing, and Evaluation. This is required since when using any classifier, the use of distinct subsets of the available data for training and testing is required to ensure generalizability. The parameters of the classifier are set with respect to the training data set, and judged versus competitors on the testing data set, and validated on the evaluation data set. To avoid over-training (i.e., memorization of features in a specific data set that are not applicable in a general manner) this succession of training steps is discontinued when the error on the validation set begins to increase significantly. We use the error on the evaluation data set as an estimate of how well we can expect our classifier to perform on new testing data as it becomes available. This estimate can be measured by 10× leave-one-out-cross-validation on the evaluation set (100× in cases of low sample number), or batch evaluation on larger data sets.

Classifiers complimentated for the instant invention include, but are not limited to, neural networks, support vector machines, genetic algorithms, kernel-based methods, and tree-based methods.

Neural Networks

One tool to use construct classifiers is that of a mapping neural network. The flexibility of neural nets to generically model data is derived through a technique of “learning”. Given a list of examples of correct input/output pairs, a neural net is trained by systematically varying its free parameters (weights) to minimize its chi-squared error in modeling the training data set. Once these optimal weights have been determined, the trained net can be used as a model of the training data set. If inputs from the training data are fed to the neural net, the net output will be roughly the correct output contained in the training data. The nonlinear interpolatory ability manifests itself when one feeds the net sets of inputs for which no examples appeared in the training data. A neural net “learns” enough features of the training data set to completely reproduce it (up to a variance inherent to the training data); the trained form of the net acts as a black box that produces outputs based on the training data.

Neural networks typically have a number of ad hoc parameters, such as selection of the number of hidden layers, the number of hidden-layer neurons, parameters associated with the learning or optimization technique used, and in many cases they require a validation set for a stopping criterion. In addition, neural network weights are trained iteratively, producing problems with convergence to local minima. We have developed several types of neural networks that solve these problems. Our solutions involve nonlinearly transforming the input pattern fed into the neural network. This transformation is equivalent to feature selection (though one still needs as many inputs into the classifier) and can be quite powerful when combined with the independent feature selection techniques previously described. For use of neural networks in medical decision support, see Barnhill et al., U.S. Pat. No. 5,769,074, filed May 3, 1996, and Astion, M. L., et al., “Application of Neural Networks to the Interpretation of Laboratory Data in Cancer Diagnosis”, Clin. Chem., vol. 38, No. 1, p. 34-38 (1992), both incorporated by reference with all tables and figures.

Genetic Algorithms

Genetic algorithms (GAs) typically maintain a constant sized population of individual solutions that represent samples of the space to be searched. Each individual is evaluated on the basis of its overall “fitness” with respect to the given application domain. New individuals (samples of the search space) are produced by selecting high performing individuals to produce “offspring” that retain features of their “parents”. This eventually leads to a population that has improved fitness with respect to the given goal.

New individuals (offspring) for the next generation are formed by using two main genetic operators: crossover and mutation. Crossover operates by randomly selecting a point in the two selected parents gene structures and exchanging the remaining segments of the parents to create new offspring. Therefore, crossover combines the features of two individuals to create two similar offspring. Mutation operates by randomly changing one or more components of a selected individual. It acts as a population perturbation operator and is a means for inserting new information into the population. This operator prevents any stagnation that might occur during the search process.

GAs have demonstrated substantial improvement over a variety of random and local search methods. This is accomplished by their ability to exploit accumulating information about an initially unknown search space in order to bias subsequent search into promising subspaces. Since GAs are basically a domain independent search technique, they are ideal for applications where domain knowledge and theory is difficult or impossible to provide. See Arouh et al., U.S. patent application Ser. No. 10/611,220, incorporated by reference, for further useage of GAs for classification.

SVMs

The key idea behind support vector machines (SVMs, Vapnik, 1995) is to map input vectors (i.e., patient-specific data) into a high dimensional space, and to construct in that space hyperplanes with a large margin. These hyperplanes can be thought of as boundaries separating the categories of the dataset, in this case response and non-response. The support vector machine solution proposes to find the hyperplane separating the classes. This plane is determined by the parameters of a decision function, which is used for classification. The SVM is based on the fact that there is a unique separating hyperplane that maximizes the margin between the classes.

The task of finding the hyperplane is reduced to minimizing the Lagrangian, a function of the margin and constraints associated with each input vector. The constraints depend only on the dot product of an input element and the solution vector. In order to minimize the Langrangian, the Lagrange multipliers must either satisfy those constraints or be exactly zero. Elements of the training set for which the constraints are satisfied are the so-called support vectors. The support vectors parameterize the decision function and lie on the boundaries of the margin separating the classes.

In many cases, SVMs are typically more accurate, give greater data understanding, and are more robust than other machine learning methods. Data understanding comes about because SVMs extract support vectors, which as described above are the borderline cases. Exhibiting such borderline cases allow us to identify outliers, to perform data cleaning, and to detect confounding factors. In addition, the margins of the training examples (how far they are from the decision boundary) provide useful information about the relevance of input variables, and allow the selection of the most predictive variable. SVMs are often successful even with sparse data (few examples), biased data (more examples of one category), redundant data (many similar examples), and heterogeneous data (examples coming from different sources). However, they are known to work poorly on discrete data.

In another preferred embodiment of the present invention, regression techniques are used to deliver a diagnostic or prognostic prediction using the markers declared previously. These are well-known by those of ordinary skill in the art, however a short discussion follows. For more detail, one is referred to Kleinbaum et al., Applied Regression Analysis and Multivariable Methods, Third Edition, Duxbury Press, 1998.

In the discussion of weighted least squares a need was found for a method to fit Y to more than one X. Further, it is common that the response variable Y is related to more than one regressor variable simultaneously. If a valid description of the relationship between Y and any of these response variables is to be obtained, all must be considered. Also, exclusion of any important regressor variables will adversely affect predictions of Y. In general, the equation to be considered becomes
Y=b 0+b 1X1+b 2X2+ . . . +KXK
The Xs may be any relevant regressor variables. Often one X is a (nonlinear) transformation of another. For example, X 2=1n (X 1).

When dealing with multiple linear regression, fits to data are no longer lines. For example, with K=2, the resulting fit would describe a plane in three dimensional space with “slopes” bhat 1 and bhat 2intersecting the Y axis at bhat 0. Beyond K=2 the resulting fit becomes difficult to visualize. The terminology regression surface is often used to describe a multiple linear regression fit.

Assumptions required for application of least squares methodology to multiple linear regression equations are similar to those cited for the simple linear case. For example, the true relationship between Y and the various Xs must be as given by the linear equation and the spread of the errors must be constant across values of all Xs. Also, a limit exists to the number of Xs that can be considered. Specifically, K+1 must be less than or equal to the sample size n for a unique set of bhats to be found.

In theory, least squares estimates of b 0, . . . , b K are found just as in the simple linear case. The estimates bhat 0, . . . , bhat K are the solution from minimizing sum (Yi−b0−b1X1i− . . . −bkXki)sup2.

The description of the resulting equations and associated summary statistics is best made using matrix algebra. The computations are best carried out using a computer.

The relationship between Y and X or Y and several Xs is not always linear in form despite transformations that can be applied to resulted in a linear relationship. In some instances such a transformation may not exist and in others theoretical concerns may require analysis to be carried out with the untransformed equation.

Least squares methodology can be used to solve nonlinear regression problems. For the above equation the least squares estimates of the parameters would be the solution of the minimization of sum(W−A(1−e sup Bt)sup C)sup 2

Application of calculus leads to three equations whose solution requires an iterative technique. For all but the simplest of cases, solving nonlinear least squares problems involves use of computer-based algorithms. A multitude of such algorithms exist emphasizing the number of problems whose valid solution requires the nonlinear least squares technique.

Several variations of nonlinear regression exist, which one of ordinary skill in the art will be aware. One preferred case in the present invention is the use of deterministic greedy algorithms for building sparse nonlinear regression models from observational data. In this embodiment, the objective is to develop efficient numerical schemes for reducing the training and runtime complexities of nonlinear regression techniques applied to massive datasets. In the spirit of Natarajan's greedy algorithm (Natarajan, 1995), the procedure is to iteratively minimize a loss function subject to a specified constraint on the degree of sparsity required of the final model or an upper bound on the empirical error. There exist various greedy criteria for basis selection and numerical schemes for improving the robustness and computational efficiency of these algorithms.

In another preferred embodiment of the present invention, a kernel-based method is trained to deliver a diagnostic or prognostic prediction using the markers declared previously. One such method is Kernel Fisher's Discriminant (KFD). Fisher's discriminant (Fisher, 1936) is a technique to find linear functions that are able to discriminate between two or more classes. Fisher's idea was to look for a direction w that separates the class means values well (when projected onto the found direction) while achieving a small variance around these means. The hope is that it is easy to differentiate between either of the two classes from this projection with a small error. The quantity measuring the difference between the means is called between class variance and the quantity measuring the variance around these class means is called within class variance, respectively. The goal is to find a direction that maximizes the between class variance while minimizing the within class variance at the same time. As this technique has been around for almost 70 years it is well known and widely used to build classifiers.

Unfortunately, as previously discussed, many biological datasets are not solvable using linear techniques. Therefore, one of the classifiers we use is a non-linear variant of Fisher's discriminant. This non-linearization is made possible through the use of kernel functions, a “trick” that is borrowed from support vector machines (Boser et al., 1992). Kernel functions represent a very principled and elegant way of formulating non-linear algorithms, and the findings that are derived from using them have clear and intuitive interpretations.

In the KFD technique (Mika, 1999), one first maps the data into some feature space F through some non-linear mapping Φ. One then computes Fisher's linear discriminant in this feature space, thus implicitly yielding a non-linear discriminant in input space. In a methodology similar to SVMs, this mapping is defined in terms of a kernel function k(x,y)=(Φ(x)·Φ(y)). The training examples (i.e. the data vector containing all marker values for each patient) can in turn be expanded in terms of this kernel function as well. From this relationship one can write a formulation of the between and within class variance in terms of dot products of the kernel function and training patterns and thus find Fisher's linear discriminant in F by maximizing the ratio of these two quantities.

In another preferred embodiment of the present invention, an algorithm using Bayesian learning is trained to deliver a diagnostic or prognostic prediction using the markers declared previously. See Pearl, J. (1988). Probabilistic Reasoning in Intelligent Systems: networks of plausible inference, Morgan Kaufinann, for an overview of Bayesian learning.

While Bayesian networks (BNs) are powerful tools for knowledge representation and inference under conditions of uncertainty, they were not considered as classifiers until the discovery that Narve-Bayes, a very simple kind of BNs that assumes the attributes are independent given the class node, are surprisingly effective. See Langley, P., Iba, W. and Thompson, K. (1992). An analysis of Bayesian classifiers. In Proceedings of AAAI-92 pp. 223-228.

A Bayesian network B is a directed acyclic graph (DAG), where each node N represents a domain variable (i.e., a dataset attribute), and each arc between nodes represents a probabilistic dependency, quantified using a conditional probability distribution (CP table) for each node n.sub.i . A BN can be used to compute the conditional probability of one node, given values assigned to the other nodes; hence, a BN can be used as a classifier that gives the posterior probability distribution of the class node given the values of other attributes. A major advantage of BNs over many other types of predictive models, such as neural networks, is that the Bayesian network structure represents the inter-relationships among the dataset attributes. One of ordinary skill in the art can easily understand the network structures and if necessary modify them to obtain better predictive models. By adding decision nodes and utility nodes, BN models can also be extended to decision networks for decision analysis. See Neapolitan, R. E. (1990), Probabilistic reasoning in expert systems: theory and algorithms, John Wiley& Sons.

Applying Bayesian network techniques to classification involves two sub-tasks: BN learning (training) to get a model and BN inference to classify instances. Learning BN models can be very efficient. As for Bayesian network inference, although it is NP-hard in general (See for instance Cooper, G. F. (1990) Computational complexity of probabilistic inference using Bayesian belief networks, In Artificial Intelligence, 42 (pp. 393-405).), it reduces to simple multiplication in a classification context, when all the values of the dataset attributes are known.

There are two ways to view a BN, each suggesting a particular approach to learning. First, a BN is a structure that encodes the joint distribution of the attributes. This suggests that the best BN is the one that best fits the data, and leads to the scoring based learning algorithms, that seek a structure that maximizes the Bayesian, MDL or Kullback-Leibler (KL) entropy scoring function. See for instance Cooper, G. F. and Herskovits, E. (1992). A Bayesian Method for the induction of probabilistic networks from data. Machine Learning, 9 (pp. 309-347). Second, the BN structure encodes a group of conditional independence relationships among the nodes, according to the concept of d-separation. See for instance Pearl, J. (1988). Probabilistic Reasoning in Intelligent Systems: networks of plausible inference, Morgan Kaufmann. This suggests learning the BN structure by identifying the conditional independence relationships among the nodes. These algorithms are referred as CI-based algorithms or constraint-based algorithms. See for instance Cheng, J., Bell, D. A. and Liu, W. (1997a). An algorithm for Bayesian belief network construction from data. In Proceedings of AI &STAT'97 (pp.83-90), Florida.

Friedman et al. (1997) show theoretically that the general scoring-based methods may result in poor classifiers since a good classifier maximizes a different function -viz., classification accuracy. Greiner et al. (1997) reach the same conclusion, albeit via a different analysis. Moreover, the scoring-based methods are often less efficient in practice. The preferred embodiment is Cl-based learning algorithms to effectively learn BN classifiers.

The present invention envisions using, but is not limited to, the following five classes of BN classifiers: Naëve-Bayes, Tree augmented Naïve-Bayes (TANs), Bayesian network augmented Naïve-Bayes (BANs), Bayesian multi-nets and general Bayesian networks (GBNs). By use of this methodology it is possible to build a predictive model of the data.

These models can be put on firm theoretical foundations of statistics and probability theory, i.e. in a Bayesian setting. The computation required for inference in these models include optimization or marginalisation over all free parameters in order to make predictions and evaluations of the model. Inference in all but the very simplest models is not analytically tractable, so approximate techniques such as variational approximations and Markov Chain Monte Carlo may be needed. Models include probabilistic kernel based models, such as Gaussian Processes and mixture models based on the Dirichlet Process.

Ensemble Networks

The final step in predictor development, assembly of committee, or ensemble, networks.It is common practice to train many different candidate networks and then to select the best, on the basis of performance on an independent validation set, for instance, and to keep this network, discarding the rest. There are two disadvantages to this approach. First, the effort involved in training the remaining networks is wasted. Second, the generalization performance on the validation set has a random component due to noise on the data, and so the network that had the best performance on the validation set might not be the one with the best performance on the new test set.

These drawbacks can be overcome by combining the networks together to form a committee. This can lead to significant improvements in the predictions on new data while involving little additional computational effort. In fact, the performance of a committee can be better than the performance of the best single network in isolation. The error due to the committee can be shown to be:
E_{COM}−1/L E_{AV}
Where L is the number of committee members and EAV the average error contributed to the prediction by a single member of the committee. Typically, some useful reduction in error is obtained, and the method is trivial to implement.

The challenging problem of integration is to decide which one(s) of the classifiers to rely on or how to combine the results produced by the base classifiers. One of the most popular and simplest techniques used is called majority voting. In the voting technique, each base classifier is considered as an equally weighted vote for that particular prediction. The classification that receives the largest number of votes is selected as the final classification (ties are solved arbitrarily). Often, weighted voting is used: each vote receives a weight, which is usually proportional to the estimated generalization performance of the corresponding classifier. Weighted Voting (WV) works usually much better than simple majority voting.

Boosting Networks

Boosting has been found to be a powerful classification technique with remarkable success on a wide variety of problems, especially in higher dimensions. It aims at producing an accurate combined classifier from a sequence of weak (or base) classifiers, which are fitted to iteratively reweighted versions of the data.

In each boosting iteration, m, the observations that have been misclassified at the previous step have their weights increased, whereas the weights are decreased for those that were classified correctly. The m^{th} weak classifier f(m) is thus forced to focus more on individuals that have been difficult to classify correctly at earlier iterations. In other words, the data is re-sampled adaptively so that the weights in the re-sampling are increased for those cases most often misclassified. The combined classifier is equivalent to a weighted majority vote of the weak classifiers.

Entropy-Based

One efficient way to construct an ensemble of diverse classifiers is to use different feature subsets. To be effective, an ensemble should consist of high-accuracy classifiers that disagree on their predictions. To measure the disagreement of a base classifier and the whole ensemble, we calculate the diversity of the base classifier over the instances of the validation set as an average difference in classifications of all possible pairs of classifiers including the given one. A measure of this is based on the concept of entropy:

$$div\_ent = \frac{1}{N} {\sum }_{l = 1}^{N} {\sum }_{k = 1}^{l} - \frac{{N}_{k}^{l}}{S} \cdot log ( \frac{{N}_{k}^{l}}{S} )$$

where N is the number of instances in the data set, S is the number of base classifiers, 1 is the number of classes, and Nₖ^{l} is the number of base classifiers that assign instance i to class k.

The foregoing and following description of the invention and the various embodiments is not intended to be limiting of the invention but rather is illustrative thereof. Those skilled in the art of molecular genetics and bioinformatics can formulate further embodiments encompassed within the scope of the present invention.

### EXAMPLES

### Example I

Bipolar Disorder Response Modeling: Lithium

Summary of Clinical Indication Dependent Genetic Lithium Response Models

Subjects:

A retrospective clinical study was completed with 184 subjects with a family history of bipolar affective disorder and a diagnosis of bipolar affective disorder. No formal cognitive, behavioral, or other psychotherapy was administered. Informed consent was obtained from all subjects after the procedure had been fully explained; subjects were unrelated and of Caucasian descent (Table 1). Eight additional co-diagnoses were also assessed: Dysphoric Mania/Mixed States, Bipolar stage, Rapid Cycling, History of Suicide, Post Traumatic Stress Disorder, Panic Attack, Panic Disorder, and Alchoholism/drug abuse.

The outcome measure for the lithium response study was a score of either strong, partial, or non-response. The measure was assessed from longitudinal patient observation for manic episodes over a period of at least 5 years. The subjects were genotyped for 86 polymorphisms, including SNPs and insertion-deletion mutations.

Methods of Analysis:

For each of the different clinical co-diagnoses, subpopulations were defined from the patients with and without the particular diagnosis. Modeling for lithium response for the treatment of patients with bipolar affective disorder was performed independently for each of the different subpopulations. The models were built and tested using 50-fold cross-validation in conjunction with machine learning algorithms: probabilistic neural networks (PNN) and decision trees. For the decision trees, the terminal node labels were reassigned during training as a discrete probability of response bin, reflecting training cross-validation response purity in a set of equal bins spanning 0 to 1. Selection of features for predicting lithium response was performed with sequential floating forward feature selection (SFFS). During SFFS, feature significance was evaluated with 5-fold cross-validation over the training set. The cost criteria used was diagnostic odds ratio for PNN and quadratic misclassification cost based on the four probability of response bins for the decision trees. For model training, each of the training set folds was re-sampled to approximate an equal responder and non-responder ratio. For the PNN, the spread parameter was optimized on each training set fold. Data analysis was performed with Matlab. When evaluating odds ratios, a “pseudo-count” of 0.5 was added to any field that was 0 to make the calculation feasible.

Independent of the machine learning, the relative risks of SNPs for lithium response as well as a trend in the probability of lithium response across allelic forms were assessed with chi squared analysis, and the statistical significance was adjusted due to multiple test comparisons by controlling for false discovery rate.

SNPs were coded as numeric values assigned according to control population frequencies in the present study: 1—homozygous minor allele; 2—heterozygous, 3—homozygous major allele; —9 ambiguous. With this coding structure, it was possible to test for trends in allelic forms. For the employed machine learning models, the SNPs are considered to be nominal data and the coding is unimportant. Ambiguous SNP values refer to data points with a poor signal to noise ratio in the genotyping assays, and are treated as missing values. Only complete data subsets consisting only of patients with no missing measurements present in the SNPs being evaluated for including in them model were used for model training and feature selection.

### Example II

Clinical Indication: No History of Suicidal Ideation—Multivariate Model

For the patient population with no clinical co-diagnosis of a history of suicidal ideation, two SNPs (Table 1) were selected by the decision tree method for inclusion in a model predicting lithium treatment response for bipolar affective disorder. One of the two SNPS selected by machine learning, rs1619120, was statistically significant when assessed by univariate chi-squared analysis for a trend in proportions (q_{FDR}=0.04). The other SNP, rs1565445, while not significant in the univariate analysis was statistically significant when the SNP rs1619120 was 2 (Table 1).

The selected SNPs were in gene NTRK2, also known as TRKB, which is the receptor for brain-derived neurotrophic factor (BDNF; 113505). Together NTRK2 and BDNF regulate both short-term synaptic functions and long-term potentiation of brain synapses. The findings of recent studies indicate that suggest that the BDNF/TrkB pathway plays an essential role in mediating the neuroprotective effect of lithium. [Hashimoto R, Takei N, Shimazu K, Christ L, Lu B, Chuang D M. Lithium induces brain-derived neurotrophic factor and activates TrkB in rodent cortical neurons: an essential step for neuroprotection against glutamate excitotoxicity. Neuropharmacology. December 2002;43(7): 1173-9.]

The model based on these two SNPs was able to provide a useful prediction of the probability of positive treatment outcome in the training data, particularly between bins 1 and 4 (Table 2). This performance was maintained within expected levels for the cross-validated results (Table 3), with an overall statistically significant result (p<0.001) for the cross-validated model performance. When comparing bin 1 to bin 4 of the predicted model response, the model achieved a sensitivity of 0.82 and a specificity of 0.93 for the patient population with no history of suicidal ideation, which had a prior probability of positive treatment response of 0.50 (Table 4). The positive and negative likelihood ratios of 11 and 0.19, respectively, were statistically significant. This test can be expected to provide useful diagnostic indication of lithium treatment outcome for those patients who do not have a history of suicidal ideation, as characterized by the overall diagnostic outcome ratio of 57 and a lower confidence bound of 10.

As expected however, when the model was applied to patients with a history of suicidal ideation, it was not able to predict treatment outcome, as summarized by the overall diagnostic outcome not different than 1 for this subpopulation (Table 4). The complete lack of diagnostic utility of the model on the non-indicated population is the lack of discrimination of the overall model output (p=0.57) (Table 5). This modeling performance agrees with the univariate analysis that found none of the screened SNPs to be statistically significant for lithium response when there was a history of suicidal ideation.

The univariate analysis also indicated three other SNPs had statistically significant associations with lithium response on an independent basis (Table 6). These SNPs were also all in the NTRK2 gene. Two of these SNPS were introns and SNP rs1378923 was in the 3′ UTR region. This latter SNP showed the best overall significant univariate relationship with lithium response. SNP 1378923 also had the highest univariate DOR for this subpopulation, and was selected by machine learning as the basis of a model for lithium response.

Several of the SNPs were in linkage disequilibrium with each other (Table 7): rs1619210, rs1187352, and rs1187356 as well as rs1565445 and 1387923. This indicates from a modeling perspective that the two selected SNPS for the present model rs1619120 and rs1565445 were not in linkage disequilibrium with each other, but that SNPs rs1187352 and rs1187356 could be substituted for SNP rs1619120 and 1387923 could be substituted for rs1565445 in a model of lithium response.

### Example III

Clinical Indication: No History of Suicidal Ideation—Alternate Univariate Model

For the patient population with no clinical co-diagnosis of a history of suicidal ideation, a second model independent of the first was suggested (Table 1) by the decision tree method for inclusion in a model predicting lithium treatment response for bipolar affective disorder. The one SNP selected by machine learning, rs1387923 (Table 8), was statistically significant when assessed by univariate chi-squared analysis for a trend in proportions (q_{FDR}=0.02).

The simple discriminate model based on this single SNP was able to provide a useful prediction of the probability of positive treatment outcome in the training set (Table 9). This performance was maintained within expected levels for the cross-validated results (Table 10), with an overall statistically significant result (p<0.001) for the cross-validated model performance. When comparing bin 1 to bin 2 of the predicted model response, the model achieved a sensitivity for positive lithium response of 0.89 but a specificity of only 0.44 for the patient population with no history of suicidal ideation (Table 11). This result indicates that the test would be most useful as a means to rule out lithium treatment. The positive and negative likelihood ratios of 1.61 and 0.24, respectively, were statistically significant. The negative likelihood ratio indicates that the test can be expected to provide some indication of poor lithium treatment outcome for those patients who do not have a history of suicidal ideation. However, this single SNP is not a highly useful standalone test for lithium response, as characterized by the overall diagnostic outcome ratio of 7 with a lower confidence interval bound of 2.5.

As expected however, when the model was applied to patients with a history of suicidal ideation, it was not able to predict treatment outcome, as summarized by the overall diagnostic outcome not different than 1 for this subpopulation (Table 11). The complete lack of diagnostic utility of the model on the non-indicated population is the lack of discrimination of the overall model output (p=0.50) (Table 12). This modeling performance agrees with the univariate analysis that found none of the screened SNPs to be statistically significant for lithium response when there was a history of suicidal ideation.

### Example IV

Clinical Indication: Co-Diagnosis of Panic Disorder

For the patient population with a co-diagnosis of Panic Disorder, a single SNP model was posed by the decision tree method for predicting lithium treatment response for bipolar affective disorder. The one SNP selected by machine learning, rs971362 (Table 13), had an unadjusted p value of 0.0075, however it was not statistically significant when adjusted for multiple comparisons by FDR (q_{FDR}=0.47).

The selected SNP was in the promoter of gene IMPA2. IMPA2 is one of the two genes encoding human IMPases, which lithium has been demonstrated to inhibit [Hallcher, 1980#142]. The consequence of IMPase inhibition is inositol depletion. The assocation of the SNP in the promoter of IMPA2 agrees with the proposed mechanism that lithium inhibits IMPases, suppressing the inositol signal transduction pathway through depletion of intracellular inositol [Search for a common mechanism of mood stabilizers. Harwood A J, Agam G. Biochem Pharmacol. 2003 Jul. 15;66(2):179-89]. Therefore, variations in the IMPase product may result in variations in the lithium response phenotype.

The simple discriminate model based on this single SNP was able to provide a useful prediction of the probability of positive treatment outcome in the training set (Table 14). This performance was maintained within expected levels for the cross-validated results (Table 15), with an overall statistically significant result (p=0.005) for the cross-validated model performance. When comparing bin 1 to bin 2 of the predicted model response, the model only achieved a sensitivity for positive lithium response of 0.60 but a specificity of 0.82 for the patient population with a co-diagnosis of Panic Disorder (Table 16). This result indicates that the test would be most useful as a means to identify likely candidates for lithium treatment, but would not be suitable conclude that the patient would not respond favorably to lithium. The positive and negative likelihood ratios of 3.3 and 0.49, respectively, were statistically significant. The positive likelihood ratio indicates that the test can be expected to provide some indication of positive lithium treatment outcome for those patients who have a co-diagnosis of Panic Disorder. However, this single SNP is not a highly useful standalone test for lithium response, as characterized by the overall diagnostic outcome ratio of 7 with a lower confidence interval bound of 1.7.

As expected, when the model was applied to patients without a co-diagnosis of Panic-Disorder, it was not able to predict treatment outcome, as summarized by the overall diagnostic outcome not different than 1 for this subpopulation (Table 16). The complete lack of diagnostic utility of the model on the non-indicated population is the lack of discrimination of the overall model output (p=0.95) (Table 17). This modeling performance agrees with the univariate analysis that found the selected SNP rs971362 unassociated for lithium response when there was not a co-diagnosis of Panic Disorder p=0.9.

### Example V

Clinical Indication: No history of Rapid Cycling

For the patient population without Rapid Cycling, a single SNP model was posed by the decision tree method for predicting lithium treatment response for bipolar affective disorder. The one SNP selected by machine learning, rs1387923 (table 18), had an unadjusted p value of 0.0075, however it was not statistically significant when adjusted for multiple comparisons by FDR (q_{FDR}=0.47).

The selected SNP was in gene NTRK2, also known as TRKB, which is the receptor for brain-derived neurotrophic factor (BDNF; 113505). Together NTRK2 and BDNF regulate both short-term synaptic functions and long-term potentiation of brain synapses. The findings of recent studies indicate that suggest that the BDNF/TrkB pathway plays an essential role in mediating the neuroprotective effect of lithium. [Hashimoto R, Takei N, Shimazu K, Christ L, Lu B, Chuang D M. Lithium induces brain-derived neurotrophic factor and activates TrkB in rodent cortical neurons: an essential step for neuroprotection against glutamate excitotoxicity. Neuropharmacology. December 2002;43(7): 1173-9.]

The simple discriminate model based on this single SNP was able to provide a useful prediction of the probability of positive treatment outcome in the training set (Table 19). This performance was maintained within expected levels for the cross-validated results (Table 20), with an overall statistically significant result (p=0.0003) for the cross-validated model performance. When comparing bin 1 to bin 2 of the predicted model response, the model only achieved a sensitivity for positive lithium response of 0.88 but a specificity of 0.43 for the patient population without Rapid Cycling (Table 21). This result indicates that the test would be most useful as a means to identify likely candidates with an expected poor response to lithium treatment, but it would not be suitable conclude that the patient would respond favorably to lithium. The positive and negative likelihood ratios of 1.6 and 0.27, respectively, were statistically significant. The negative likelihood ratio indicates that the test can be expected to provide some indication of poor lithium treatment outcome for those patients who do not have Rapid Cycling. However, this single SNP is not a highly useful standalone test for lithium response, as characterized by the overall diagnostic outcome ratio of 6 with a lower confidence interval bound of 2.1.

As expected, when the model was applied to patients with Rapid Cycling, it was not able to predict treatment outcome, as summarized by the overall diagnostic outcome not different than 1 for this subpopulation (Table 21). The complete lack of diagnostic utility of the model on the non-indicated population is the lack of discrimination of the overall model output (p=0.75) (Table 22). This modeling performance agrees with the univariate analysis that found a quite dissimilar association of the SNP's allelic forms with lithium response in the cases of the presence and absence of rapid cycling (Table 23).

### Example VI

Clinical Indication: No history of Dysphoric Mania/Mixed States

For the patient population without Dysphoric Mania/Mixed States, a single SNP model was posed by the decision tree method for predicting lithium treatment response for bipolar affective disorder. The one SNP selected by machine learning, rs1387923 (Table 24), had an unadjusted p value of 0.0003 and was statistically significant when assessed by univariate chi-squared analysis for a trend in proportions (q_{FDR}=0.02).

The selected SNP was in gene NTRK2, also known as TRKB, which is the receptor for brain-derived neurotrophic factor (BDNF; 113505). Together NTRK2 and BDNF regulate both short-term synaptic functions and long-term potentiation of brain synapses. The findings of recent studies indicate that suggest that the BDNF/TrkB pathway plays an essential role in mediating the neuroprotective effect of lithium. [Hashimoto R, Takei N, Shimazu K, Christ L, Lu B, Chuang D M. Lithium induces brain-derived neurotrophic factor and activates TrkB in rodent cortical neurons: an essential step for neuroprotection against glutamate excitotoxicity. Neuropharmacology. December 2002;43(7):1173-9.]

The discriminate model based on this single SNP was able to provide a useful prediction of the probability of positive treatment outcome in the training set (Table 25). This performance was maintained within expected levels for the cross-validated results (Table 26), with an overall statistically significant result (p=0.0013) for the cross-validated model performance. When comparing bin 1 to bin 2 of the predicted model response, the model only achieved a sensitivity for positive lithium response of 0.85 but a specificity of 0.41 for the patient population without Dys Mania/Mixed States (Table 27). This result indicates that the test would be most useful as a means to identify likely candidates with an expected poor response to lithium treatment, but it would not be suitable conclude that the patient would respond favorably to lithium. The positive and negative likelihood ratios of 1.4 and 0.37, respectively, were statistically significant. The negative likelihood ratio indicates that the test can be expected to provide some indication of poor lithium treatment outcome for those patients who do not have Dys Mania/Mixed States. However, this single SNP is not a highly useful standalone test for lithium response, as characterized by the overall diagnostic outcome ratio of 4 with a lower confidence interval bound of 1.7.

As expected, when the model was applied to patients with Dys Mania/Mixed States, it was not able to predict treatment outcome, as summarized by the overall diagnostic outcome not different than 1 for this subpopulation (Table 27). The complete lack of diagnostic utility of the model on the non-indicated population is the lack of discrimination of the overall model output (p=0.60) (Table 28). This modeling performance agrees with the univariate analysis that found no statistically significant association of the surveyed SNPs with lithium response in the cases of the presence of Dys Mania/Mixed States.

### Example VII

Clinical Indication: Panic Disorder (Negative Co-diagnosis)

For the patient population without a co-diagnosis of Panic Disorder, a single SNP model was posed by the decision tree method for predicting lithium treatment response for bipolar affective disorder. The one SNP selected by machine learning, rs971363 (Table 29), had an unadjusted p value of 0.0077, however it was not statistically significant when adjusted for multiple comparisons by FDR (q_{FDR}=0.19).

The selected SNP was in the promoter of gene IMPA2. IMPA2 is one of the two genes encoding human IMPases, which lithium has been demonstrated to inhibit [Hallcher, 1980#142]. The consequence of IMPase inhibition is inositol depletion. The assocation of the SNP in the promoter of IMPA2 agrees with the proposed mechanism that lithium inhibits IMPases, suppressing the inositol signal transduction pathway through depletion of intracellular inositol [Search for a common mechanism of mood stabilizers. Harwood A J, Agam G. Biochem Pharmacol. 2003 Jul. 15;66(2):179-89]. Therefore, variations in the IMPase product may result in variations in the lithium response phenotype.

The simple discriminate model based on this single SNP was able to provide a useful prediction of the probability of positive treatment outcome in the training set (Table 30). This performance was maintained within expected levels for the cross-validated results (Table 31), with an overall statistically significant result (p=0.008) for the cross-validated model performance. When comparing bin 1 to bin 2 of the predicted model response, the model only achieved a sensitivity for positive lithium response of only 0.29 but a specificity of 0.90 for the patient population with a co-diagnosis of Panic Disorder (Table 32). This result indicates that the test would be most useful as a means to identify likely candidates for lithium treatment, but would not be suitable conclude that the patient would not respond favorably to lithium. The positive likelihood ratio of 2.7 was statistically significant. The positive likelihood ratio indicates that the test can be expected to provide some indication of positive lithium treatment outcome for those patients who do not have a co-diagnosis of Panic Disorder. However, this single SNP is not a highly useful standalone test for lithium response, as characterized by the overall diagnostic outcome ratio of 3 with a lower confidence interval bound of 1.3.

As expected, when the model was applied to patients with a co-diagnosis of Panic-Disorder, it was not able to predict treatment outcome, as summarized by the overall diagnostic outcome not different than 1 for this subpopulation (Table 32). The complete lack of diagnostic utility of the model on the non-indicated population is the lack of discrimination of the overall model output (p=0.81) (Table 33). This modeling performance agrees with the univariate analysis that found the selected SNP rs971363 unassociated for lithium response when there was a co-diagnosis of Panic Disorder.

### Example VIII

Clinical Indication: Panic Disorder (Composite Model Negative and Positive Co-diagnosis)

For the patient population with and without a co-diagnosis of Panic Disorder, a multi-SNP model was posed by the decision tree method for predicting lithium treatment response for bipolar affective disorder when the co-diagnosis was included as a modeling factor. Of the four SNPs (Table 34) selected by machine learning, the two primary SNPs were rs971362 and rs971363, as previously discussed in examples 3 and 6, respectively.

An additional selected SNP rs972691 was an intron of gene INPP1. INPP1 is a gene encoding human IPPase, which lithium has been demonstrated to inhibit [Inhorn, 1988#143] much like the case of the other IMPases, the consequence of IPPase inhibition is inositol depletion. The assocation of the SNP in the intron of INPP1 in conjunction with the others in IMPA2 agrees with the proposed mechanism that lithium inhibits IMPases and IPPase, suppressing the inositol signal transduction pathway through depletion of intracellular inositol [Search for a common mechanism of mood stabilizers. Harwood A J, Agam G. Biochem Pharmacol. 2003 Jul. 15;66(2):179-89]. Therefore, variations in the both the IMPase and IPPase product may result in variations in the lithium response phenotype.

The other selected SNP was rs2049045 that is in gene BDNF, the receptor for brain-derived neurotrophic factor (BDNF; 113505). Together NTRK2 and BDNF regulate both short-term synaptic functions and long-term potentiation of brain synapses. The findings of recent studies indicate that suggest that the BDNF/TrkB pathway plays an essential role in mediating the neuroprotective effect of lithium. [Hashimoto R, Takei N, Shimazu K, Christ L, Lu B, Chuang D M. Lithium induces brain-derived neurotrophic factor and activates TrkB in rodent cortical neurons: an essential step for neuroprotection against glutamate excitotoxicity. Neuropharmacology. December 2002;43(7):1173-9.] This is a complimentary pathway to the IMPase/IPPase pathway previously implicated in relation to patients with and without a co-diagnosis of Panic Disorder.

The constructed discriminate model based on this set of SNPs and the clinical co-diagnosis was able to provide a useful prediction of the probability of positive treatment outcome in the training set (Table 35). This performance was maintained within expected levels for the cross-validated results (Table 36), with an overall statistically significant result (p=<1e−6) for the cross-validated model performance. When comparing bin 1 to bin 4 of the predicted model response, the model only achieved a sensitivity for positive lithium response of only 0.48 but a specificity of 0.98 for the patient population with a co-diagnosis of Panic Disorder (Table 37). The positive and negative likelihood ratios of 21 and 0.53, respectively, were statistically significant. The positive likelihood ratio indicates that the test can be expected to provide a good indication of positive lithium treatment outcome, but the relatively high negative likelihood ratio indicates that the test is not sufficient to rule out non-responders. Overall, this single SNP is moderately useful standalone test for lithium response, as characterized by the overall diagnostic outcome ratio of 39 with a lower confidence interval bound of 4.5.

### Example IX

Clinical Indication: PTSD (Negative Co-diagnosis)

For the patient population without a co-diagnosis of PTSD, a multi-SNP model was posed by the decision tree method for predicting lithium treatment response for bipolar affective disorder. The three SNPs selected by machine learning, were not independently statistically significant when adjusted for multiple comparisons by FDR (Table 38).

The three SNPs selected were two introns in NTRK2 and one intron in INPP1. INPP1 is a gene encoding human IPPase, which lithium has been demonstrated to inhibit [Inhorn, 1988#143] much like the case of the other IMPases, the consequence of IPPase inhibition is inositol depletion. The other selected SNP was an intron in the gene NTRK2, also known as TRKB, which is the receptor for brain-derived neurotrophic factor (BDNF; 113505). Together NTRK2 and BDNF regulate both short-term synaptic functions and long-term potentiation of brain synapses. The findings of recent studies indicate that suggest that the BDNF/TrkB pathway plays an essential role in mediating the neuroprotective effect of lithium. [Hashimoto R, Takei N, Shimazu K, Christ L, Lu B, Chuang D M.

The discriminate model based on this SNP set and no PTSD co-diagnosis was able to provide a useful prediction of the probability of positive treatment outcome in the training set (Table 39). This performance was maintained within expected levels for the cross-validated results (Table 40), with an overall statistically significant result (p=0.008) for the cross-validated model performance. When comparing bin 1 to bin 3 of the predicted model response, the model only achieved a sensitivity for positive lithium response of only 0.47 but a specificity of 0.89 for the patient population without a co-diagnosis of PTSD (Table 41). The positive likelihood ratio of 4.1 was statistically significant. This result indicates that the test would be most useful as a means to identify likely candidates for lithium treatment, but would not be suitable conclude that the patient would not respond favorably to lithium. However, this single SNP is not a highly useful standalone test for lithium response, as characterized by the overall diagnostic outcome ratio of 7 with a lower confidence interval bound of 2.2.

As expected, when the model was applied to patients with a co-diagnosis of PTSD, it was not able to predict treatment outcome, as summarized by the overall diagnostic outcome not different than 1 for this subpopulation (Table 41), (Table 42).

### Example X

Clinical Indication: Hierarchical Model for patients with assessed diagnosis for Panic Disorder and with No History of Suicidal Ideation

For the patient population with an assessed co-diagnosis of Panic Disorder, and PTSD and Suicidal Ideation, a conditional model was constructed from the 50× cross validation results of two decision tree models for predicting lithium treatment response for bipolar affective disorder. The first of the two decision tree models was obtained for the subpopulation of patients with no history of suicidial ideation and no co-diagnosis of PTSD (submodel 1). The second was obtained for those with an assessed diagnosis of panic disorder, either positive or negative (submodel 2). The decision tree model for the patients with an assessed co-diagnosis of panic disorder is discussed in EXAMPLE VIII. The decision tree model for the patients with no history of suicidial ideation and a negative co-diagnosis of panic disorder is formed from the same SNPs as the model for a negative co-diagnosis of panic disorder in EXAMPLE IX, but the population is different.

These two models were combined by using a simple score matrix that weighted the two models predictions in a symmetric fashion. This score matrix (table 43) also penalizes unconfirmed predictions in the case that either of the two submodels is unable to produce a prediction.

The model based on this scored multi-SNP and co-diagnosis model was able to provide a useful prediction of the probability of positive treatment outcome in the training set (Table 44).

This performance was maintained within expected levels for the cross-validated results (Table 45), with an overall statistically significant result (p<1e−4) for the cross-validated model performance. When comparing bin 1-2 to bin 4 of the predicted model response, the model achieved a sensitivity for positive lithium response of only 0.86 with a specificity of 0.78 (Table 46). The corresponding, positive and negative likelihood ratios of 4.0 and 0.18 were statistically significant. The positive likelihood ratio indicates that the test can be expected to provide an indication of positive lithium treatment outcome for patients with a model response of either 1 or 2, while the negative likelihood ratio indicates that the test can be expected to provide an indication of poor responders to lithium for patients with a model response of 4. This mutli-SNP model is useful as a standalone test for lithium response with model scores of 1,2 or 4, as characterized by the overall diagnostic outcome ratio of 22 with a lower confidence interval bound of 7.

Unfortunately, bin 3 of the model contains nearly twice the number of each of the other bins and provides a small odds ratio compared to the average response rate. When combining bin 3 with bin 4, the resulting model prediction bins 1-2 vs 3-4 yields a sensitivity of 0.51 and a specificity of 0.91. The corresponding positive and negative likelihood ratios of 5.6 and 0.54 indicate that when the test indicates a bin 1-2 outcome, that the patient is a good lithium candidate, but that the test is unable to identify which patients will respond poorly based on a bin 3-4 outcome.

Table 48 details the SNPs and genes that were included in the composite model.

### Example XI

Clinical Indication: General Hierarchical Model

For the patient population with an assessed co-diagnosis of Panic Disorder, and PTSD and Suicidal Ideation, as well as Rapid Cycling and Dys Mania/Mixed States, a decision tree model was constructed from the 50× cross validation results of four decision tree models for predicting lithium treatment response for bipolar affective disorder. The first of the two decision tree models was obtained for the subpopulation of patients with no history of suicidial ideation and no co-diagnosis of PTSD (submodel 1). The second was obtained for those with an assessed diagnosis of panic disorder, either positive or negative (submodel 2). The decision tree model for the patients with an assessed co-diagnosis of panic disorder is discussed in EXAMPLE VIII. The decision tree model for the patients with no history of suicidial ideation and a negative co-diagnosis of panic disorder is formed from the same SNPs as the model for a negative co-diagnosis of panic disorder in EXAMPLE IX, but the population is different. The third decision tree submodel was based on patients with no co-diagnosis of Rapid Cycling, as discussed in EXAMPLE V, and the fourth decision tree submodel was based on patients with no co-diagnosis of Dysphoric Mania/Mixed States, as discussed in EXAMPLE VI.

These four models were combined using a decision tree model based on their individual results by building a decision tree for the ambiguous case of bin 3 achieved when combining submodels 1 and 2. The decision tree model was built using 50× crossvalidation as before to form a hierarchical model of the other SNP and co-diagnosis based models using submodels 3 and 4. The 50× crossvalidation results are shown in Table 49.

This performance was maintained within expected levels for the cross-validated results (Table 3), with an overall statistically significant result (p<1e−4) for the cross-validated model performance. When comparing bin 1-2 to bins 3.4 and 4 of the predicted model response, the model achieved a sensitivity for positive lithium response of only 0.59 with a specificity of 0.90 (Table 50). The corresponding, positive and negative likelihood ratios of 5.6 and 0.46 were statistically significant. The positive likelihood ratio indicates that the test can be expected to provide an indication of positive lithium treatment outcome for patients with a model response of either 1 or 2, while the negative likelihood ratio indicates that the test can be expected to provide a moderate indication of poor responders to lithium for patients with a model response of 3.5 or 4. This mutli-SNP model is useful as a standalone test for lithium response with model scores of 1,2 or 3.5-4, as characterized by the overall diagnostic outcome ratio of 12.5 with a lower confidence interval bound of 5.

Unfortunately, bin 2.5 and 3.5 still provide only a relatively small odds ratio compared to the average response rate, but the overall result is improved over not including the additional diagnoses of Rapid Cycling and Dysphoric Mania/Mixed States and the one additional SNP.

Thus the overall composite model provides a probability of positive lithium response from 19% to >90% in 5 bins (Table 49). The power of the model is most demonstrable when comparing bins 1-2 vs 4, as shown in Table 51, and discussed in EXAMPLE IX. The composite model contains the following genes and SNPs (Table 52).

Further illustrative examples may be found in Ser. No. 10/951,085.

While the invention has been described and exemplified in sufficient detail for those skilled in this art to make and use it, various alternatives, modifications, and improvements should be apparent without departing from the spirit and scope of the invention.

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

APPENDIX I
SNPs OF GENES SELECTED FOR ALGORITHM DEVELOPMENT
IN DETERMINATION OF RESPONSE TO LITHIUM

| | | | Distance from | | |
|---|---|---|---|---|---|
| SNP rs# | SNP position | Band | previous SNP | Alleles | Gene(s) |
| rs1988253 | chr22:24286601 | 22q11.23 | 12263568 | A/T | ADRBK2 |
| rs1158615 | chr11:27667984 | 11p14.1 | 17167 | A/G | BDNF |
| rs1491850 | chr11:27706301 | 11p14.1 | 15247 | C/T | BDNF |
| rs1491851 | chr11:27709339 | 11p14.1 | 2314 | C/T | BDNF |
| rs1967554 | chr11:27676135 | 11p14.1 | 8151 | A/C | BDNF |
| rs2030323 | chr11:27685115 | 11p14.1 | 1624 | G/T | BDNF |
| rs2030324 | chr11:27683491 | 11p14.1 | 7356 | C/T | BDNF |
| rs2049045 | chr11:27650817 | 11p14.1 | 14325 | C/G | BDNF |
| rs2203877 | chr11:27627486 | 11p14.1 | 3340885 | C/T | BDNF |
| rs6265 | chr11:27636492 | 11p14.1 | 9006 | A/G | BDNF |
| rs727155 | chr11:27707025 | 11p14.1 | 724 | C/T | BDNF |
| rs962368 | chr11:27691054 | 11p14.1 | 5939 | A/G | BDNF |
| rs2199503 | chr3:121261179 | 3q13.33 | 241908 | A/G | GSK3B |
| rs334559 | chr3:121292295 | 3q13.33 | 31116 | A/G | GSK3B |
| rs1967328 | chr8:82746326 | 8q21.13 | 13381 | A/C | IMPA1 |
| rs204782 | chr8:82750696 | 8q21.13 | 773 | A/G | IMPA1 |
| rs2268431 | chr8:82749923 | 8q21.13 | 591 | A/G | IMPA1 |
| rs2268432 | chr8:82749332 | 8q21.13 | 3006 | A/C | IMPA1 |
| rs2955008 | chr8:82764401 | 8q21.13 | 13705 | G/T | IMPA1 |
| rs915 | chr8:82732945 | 8q21.13 | 55023606 | C/T | IMPA1 |
| rs1962913 | chr18:12007263 | 18p11.21 | 2642 | A/G | IMPA2 |
| rs2075824 | chr18:11971208 | 18p11.21 | 590 | C/T | IMPA2 |
| rs2075826 | chr18:12018249 | 18p11.21 | 10986 | A/G | IMPA2 |
| rs3786285 | chr18:11998847 | 18p11.21 | 2530 | C/T | IMPA2 |
| rs594235 | chr18:12023033 | 18p11.21 | 49 | C/T | IMPA2 |
| rs613993 | chr18:12018580 | 18p11.21 | 331 | A/G | IMPA2 |
| rs640088 | chr18:12022984 | 18p11.21 | 4404 | C/T | IMPA2 |
| rs644004 | chr18:12004621 | 18p11.21 | 5774 | A/G | IMPA2 |
| rs644710 | chr18:11973680 | 18p11.21 | 2472 | A/G | IMPA2 |
| rs650727 | chr18:11979969 | 18p11.21 | 6289 | C/T | IMPA2 |
| rs671470 | chr18:11991419 | 18p11.21 | 11450 | A/G | IMPA2 |
| rs873086 | chr18:11996317 | 18p11.21 | 4898 | A/C | IMPA2 |
| rs971362 | chr18:11970618 | 18p11.21 | 158 | A/C | IMPA2 |
| rs971363 | chr18:11970460 | 18p11.21 | | A/C | IMPA2 |
| rs1108939 | chr2:191059696 | 2q32.2 | 6435 | G/T | INPP1 |
| rs2016037 | chr2:191042763 | 2q32.2 | 7539 | A/G | INPP1 |
| rs2067419 | chr2:191052579 | 2q32.2 | 7459 | A/G | INPP1 |
| rs2067421 | chr2:191052741 | 2q32.2 | 162 | G/T | INPP1 |
| rs3791809 | chr2:191035224 | 2q32.2 | 69742929 | C/T | INPP1 |
| rs3791815 | chr2:191045120 | 2q32.2 | 2357 | A/G | INPP1 |
| rs972691 | chr2:191053261 | 2q32.2 | 520 | A/G | INPP1 |
| rs12223 | chr6:114290577 | 6q21 | 3213 | A/G | MARCKS |
| rs2169507 | chr6:114293269 | 6q21 | 1494 | C/T | MARCKS |
| rs352082 | chr6:114287339 | 6q21 | 1273 | C/G | MARCKS |
| rs352090 | chr6:114286066 | 6q21 | 9921 | G/T | MARCKS |
| rs3734457 | chr6:114287364 | 6q21 | 25 | C/T | MARCKS |
| rs476354 | chr6:114291775 | 6q21 | 1198 | C/T | MARCKS |
| rs548031 | chr6:114276145 | 6q21 | 29405955 | A/G | MARCKS |
| rs3732359 | chr3:121019119 | 3q13.33 | 6725850 | A/G | NR1I2 |
| rs3732360 | chr3:121019271 | 3q13.33 | 152 | C/T | NR1I2 |
| rs1036914 | chr9:84667713 | 9q21.33 | 8869 | C/T | NTRK2 |
| rs1147198 | chr9:84504902 | 9q21.33 | 1740501 | A/C | NTRK2 |
| rs1187287 | chr9:84644348 | 9q21.33 | 5483 | C/T | NTRK2 |
| rs1187350 | chr9:84524791 | 9q21.33 | 1780 | C/T | NTRK2 |
| rs1187352 | chr9:84523011 | 9q21.33 | 204 | A/G | NTRK2 |
| rs1187353 | chr9:84522807 | 9q21.33 | 5532 | C/T | NTRK2 |
| rs1187356 | chr9:84534811 | 9q21.33 | 3061 | A/T | NTRK2 |
| rs1211166 | chr9:84515546 | 9q21.33 | 3465 | A/G | NTRK2 |
| rs1212171 | chr9:84512081 | 9q21.33 | 7179 | C/T | NTRK2 |
| rs1387923 | chr9:84870190 | 9q21.33 | 2130 | C/T | NTRK2 |
| rs1443444 | chr9:84638865 | 9q21.33 | 21689 | C/T | NTRK2 |
| rs1490403 | chr9:84868060 | 9q21.33 | 928 | A/T | NTRK2 |
| rs1490404 | chr9:84852084 | 9q21.33 | 5459 | C/T | NTRK2 |
| rs1565445 | chr9:84846625 | 9q21.33 | 58777 | C/T | NTRK2 |
| rs1573219 | chr9:84617176 | 9q21.33 | 53880 | C/T | NTRK2 |
| rs1619120 | chr9:84531750 | 9q21.33 | 6959 | C/T | NTRK2 |
| rs1624327 | chr9:84658844 | 9q21.33 | 7659 | C/T | NTRK2 |
| rs1778934 | chr9:84554176 | 9q21.33 | 19365 | A/G | NTRK2 |
| rs2489162 | chr9:84563296 | 9q21.33 | 9120 | C/T | NTRK2 |
| rs2808707 | chr9:84787848 | 9q21.33 | 18630 | G/T | NTRK2 |
| rs3739570 | chr9:84867132 | 9q21.33 | 15048 | C/T | NTRK2 |
| rs3739804 | chr9:84651185 | 9q21.33 | 6837 | A/G | NTRK2 |
| rs763623 | chr9:84769218 | 9q21.33 | 1328 | A/G | NTRK2 |
| rs920776 | chr9:84767890 | 9q21.33 | 100177 | C/T | NTRK2 |
| rs993315 | chr9:84517275 | 9q21.33 | 1729 | C/T | NTRK2 |

1. A method of determining a probability of response of a subject of known clinical history to a pharmaceutical agent for a mood disorder, the method comprising:
correlating (i) a mutational burden at one or more nucleotide positions in genes drawn from the group consisting essentially of ADRBK2, BNDF, GSK3B, GRK3, IMPA1, IMPA2, INPP1, MARCKS, NTRK2 and/or NR1I2 within a sample taken from the subject of known clinical history with (ii) a mutational burden at one or more corresponding nucleotide positions in a control sample having known clinical history and response outcomes to the pharmaceutical agent; and
determining from the correlating the probability of response of the subject to the pharmaceutical agent.

2. The method according to claim 1 wherein the mutational burden consists essentially of a mutation in
the BNDF gene at nucleotide position given by the RS# and genetic position 2049045 and chr11:27650817,
the IMPA2 gene at nucleotide position given by the RS# and genetic position 971362 and chr18:11970618,
the IMPA2 gene at nucleotide position given by the RS# and genetic position 971363 and chr18:11970460,
the INPP1 gene at nucleotide position given by the RS# and genetic position 972691 and chr2:191053261,
the INPP1 gene at nucleotide position given by the RS# and genetic position 2016037 and chr2:191042763,
the NTRK2 gene at nucleotide position given by the RS# and genetic position 1619120 and chr9:84531750,
the NTRK2 gene at nucleotide position given by the RS# and genetic position 1565445 and chr9:84846625,
mutations in linkage disequilibrium with any of the aforementioned nucleotides,
or combinations thereof.

3. The method according to claim 1 wherein the mutational burden consists essentially of a mutation in:
the BNDF gene at nucleotide position given by the RS# and genetic position 2049045 and chr11:27650817,
the IMPA2 gene at nucleotide position given by the RS# and genetic position 971362 and chr18:11970618,
the IMPA2 gene at nucleotide position given by the RS# and genetic position 971363 and chr18:11970460,
the INPP1 gene at nucleotide position given by the RS# and genetic position 972691 and chr2:191053261,
the INPP1 gene at nucleotide position given by the RS# and genetic position 2016037 and chr2: 191042763,
the NTRK2 gene at nucleotide position given by the RS# and genetic position 1619120 and chr9:84531750,
the NTRK2 gene at nucleotide position given by the RS# and genetic position 1565445 and chr9:84846625,
the NTRK2 gene at nucleotide position given by the RS# and genetic position 1387923 and chr9:84870190, and
mutations in linkage disequilibrium with any of the aforementioned nucleotides,
or combinations thereof.

4. The method according to claim 1 wherein the clinical history of the subject comprises:
a clinical history including at least one of previous experience of suicidal ideation, post-traumatic stress disorder, panic disorder, rapid cycling, or disphoria.

5. The method according to claim 1, wherein the correlating comprises:
predetermining a sequence of one or more of the genes BDNF, IMPA2, INPP1, or NTRK2 from humans known to be responsive or non-responsive to mood disorder medications,
comparing the predetermined sequence to that of a corresponding wildtype sequence of the BDNF, IMPA2, INPP1, or NTRK2 gene(s); and
training a computer algorithm consisting of executable computer code residing on a memory medium to identify mutations in the subjects which correlate with the response or non-response to mood disorder medications;
wherein the computer algorithm selects only subject mutations that show discriminating power, respectively.

6. The method according to claim 1, wherein the pharmaceutical agent consists essentially of:
a medication for bipolar disorder.

7. The method according to claim 5 wherein the mutational burden consists essentially of:
the BNDF gene at nucleotide position given by the RS# and genetic position 2049045 and chr11:27650817,
the IMPA2 gene at nucleotide position given by the RS# and genetic position 971362 and chr18:11970618,
the IMPA2 gene at nucleotide position given by the RS# and genetic position 971363 and chr18:11970460,
the INPP1 gene at nucleotide position given by the RS# and genetic position 972691 and chr2:191053261,
the INPP1 gene at nucleotide position given by the RS# and genetic position 2016037 and chr2: 191042763,
the NTRK2 gene at nucleotide position given by the RS# and genetic position 1619120 and chr9:84531750,
the NTRK2 gene at nucleotide position given by the RS# and genetic position 1565445 and chr9:84846625,
the NTRK2 gene at nucleotide position given by the RS# and genetic position 1387923 and chr9:84870190, and
mutations in linkage disequilibrium with any of the aforementioned nucleotides,
or combinations thereof;
and wherein the training of the computer algorithm on the mutational burden comprises the steps of
obtaining numerous examples of (i) mutational genomic data, and (ii) historical clinical history corresponding to the mutational genomic data;
constructing a algorithm suitable to map (i) the mutational genomic data as inputs to the algorithm to (ii) historical clinical results as outputs of the algorithm;
exercising the constructed algorithm to so map (i) the mutational genomic data as inputs to (ii) the historical clinical results as outputs, receiving as output values from the computer-based constructed algorithm which output values concur with the probability of occurrence of the clinical results, and transmitting the outputs to an output value receiver connected to a display; and
conducting an automated procedure to vary a mapping function, inputs to outputs, of the constructed and exercised algorithm in order that, by minimizing an error measure of the mapping function, a more optimal algorithm mapping architecture is realized;
wherein realization of the more optimal algorithm mapping architecture, also known as feature selection, means that any irrelevant inputs are effectively excised, meaning that the more optimally mapping algorithm will substantially ignore input alleles and/or said mutational pattern genomic data that is irrelevant to output clinical results; and
wherein realization of the more optimal algorithm mapping architecture, also known as feature selection, also means that any relevant inputs are effectively identified, making that the more optimally mapping algorithm will serve to identify, and use, those input alleles and/or mutational genomic data that is relevant, in combination, to output clinical results that would result in a clinical detection of disease, disease diagnosis, disease prognosis, or treatment outcome or a combination of any two, three or four of these actions.

8. The method according to claim 7 wherein the constructing is of an algorithm drawn from the group consisting essentially of
linear or nonlinear regression algorithms,
linear or nonlinear classification algorithms,
ANOVA,
neural network algorithms,
genetic algorithms,
support vector machines algorithms,
hierarchical analysis or clustering algorithms,
hierarchical algorithms using decision trees,
kernel based machine algorithms including kernel partial least squares algorithms,
kernel matching pursuit algorithms,
kernel fisher discriminate analysis algorithms,
kernel principal components analysis algorithms,
Bayesian probability function algorithms,
Markov Blanket algorithms,
a plurality of algorithms arranged in a committee network, and
forward floating search or backward floating search algorithms.

9. The method according to claim 7, wherein the realization of the more optimal feature mapping architecture, also known as feature selection, employs an algorithm drawn from the group consisting essentially of
linear or nonlinear regression algorithms,
linear or nonlinear classification algorithms,
ANOVA,
neural network algorithms,
genetic algorithms,
support vector machines algorithms,
hierarchical analysis or clustering algorithms,
hierarchical algorithms using decision trees,
kernel based machine algorithms including kernel partial least squares algorithms,
kernel matching pursuit algorithms,
kernel fisher discriminate analysis algorithms,
kernel principal components analysis algorithms,
Bayesian probability function algorithms,
Markov Blanket algorithms,
a plurality of algorithms arranged in a committee network, and
forward floating search or backward floating search algorithms.

10. The method according to claim 7 wherein a tree algorithm, including a CART or a MARS algorithm, is trained to reproduce the performance of another machine-learning classifier or regressor by enumerating the input space of said classifier or regressor to form a plurality of training examples sufficient (1) to span the input space of said classifier or regressor and (2) train the tree to emulate the performance of said classifier or regressor.

11. The method according to claim 1 wherein the pharmaceutical agent for which response is determined consists essentially of
lithium.

12. The method according to claim 1 where the pharmaceutical agent for which response is determined is drawn from the group of mood disorder medications consisting essentially of
molecular depakote,
olanzapine, and
lamotrigine.

13. The method of claim 1 wherein the at least one mutation in the mutational burden is from a group of mutations consisting essentially of
a silent mutation.
missense mutation,
or combination thereof.

14. The method according to claim 1 wherein the subject sample is selected from the group consisting of
a blood sample,
a serum sample,
a urine sample,
a tissue sample,
a saliva sample, and
a plasma sample.

15. The method according to claim 1 exercised on a mutation detected by a detection technique selected from the group consisting essentially of
hybridization with oligonucleotide probes,
a ligation reaction,
a polymerase chain reaction and single nucleotide primer-guided extension assays, and
variations thereof.

16. The method according to claim 1 wherein said correlating comprises:
comparing said mutational burden to a second mutational burden measured in a second sample obtained from another, second, subject;
wherein, when the second mutational burden of the second subject is of the type correlated with the mutational burden, then the second subject is diagnosed as being responsive or resistant to mood disorder medication.

17. The method according to claim 16 wherein the correlating is in accordance with an algorithm drawn from the group consisting essentially of
linear or nonlinear regression algorithms,
llinear or nonlinear classification algorithms,
ANOVA,
neural network algorithms,
genetic algorithms,
support vector machines algorithms,
hierarchical analysis or clustering algorithms,
hierarchical algorithms using decision trees,
kernel based machine algorithms including kernel partial least squares algorithms,
kernel matching pursuit algorithms,
kernel fisher discriminate analysis algorithms,
kernel principal components analysis algorithms,
Bayesian probability function algorithms,
Markov Blanket algorithms,
a plurality of algorithms arranged in a committee network, and
forward floating search or backward floating search algorithms.

18. The method according to claim 16 that, after the determining step, comprises:
obtaining a second sample for the subject prior to any treatment with a mood disorder medication.

19. A method for detecting the presence of, or the risk of developing, post-traumatic stress disorder in a human, said method comprising:
determining in a biological sample from the human the presence of a nucleic acid sequence having a mutational burden relating to a mutation in genes of the group consisting essentially of
the BNDF gene at nucleotide position given by the RS# and genetic position 2049045 and chr11:27650817.
the IMPA2 gene at nucleotide position given by the RS# and genetic position 971362 and chr18:11970618,
the IMPA2 gene at nucleotide position given by the RS# and genetic position 971363 and chr18:11970460,
the INPP1 gene at nucleotide position given by the RS# and genetic position 972691 and chr2:191053261,
the INPP1 gene at nucleotide position given by the RS# and genetic position 2016037 and chr2:191042763,
the NTRK2 gene at nucleotide position given by the RS# and genetic position 1619120 and chr9:84531750,
the NTRK2 gene at nucleotide position given by the RS# and genetic position 1565445 and chr9:84846625,
the NTRK2 gene at nucleotide position given by the RS# and genetic position 1387923 and chr9:84870190, and
mutations in linkage disequilibrium with any of the aforementioned nucleotides,
or combinations thereof; and
finding one or more nucleotide positions in a sequence region corresponding to a wildtype genomic DNA sequence; and
comparing the determined nucleic acid sequence having the mutational burden to the wildtype genomic DNA sequence to detect the presence of, or the risk of developing, post-traumatic stress disorder in the human.

20. A method for evaluating a compound for use in diagnosis or treatment of bipolar disorder, the method comprising:
contacting a predetermined quantity of the compound with cultured cybrid cells or animal model having genomic DNA originating from a neuronal rho or human embryonic immortal kidney cell line and from tissue of a human having both
(1) a disorder that is associated with bipolar disorder and,
(2) a mutational burden relating to a mutation in genes of the group consisting essentially of
the BNDF gene at nucleotide position given by the RS# and genetic position 2049045 and chr11:27650817.
the IMPA2 gene at nucleotide position given by the RS# and genetic position 971362 and chr18:11970618,
the IMPA2 gene at nucleotide position given by the RS# and genetic position 971363 and chr18:11970460,
the INPP1 gene at nucleotide position given by the RS# and genetic position 972691 and chr2:191053261,
the INPP1 gene at nucleotide position given by the RS# and genetic position 2016037 and chr2:191042763,
the NTRK2 gene at nucleotide position given by the RS# and genetic position 1619120 and chr9:84531750,
the NTRK2 gene at nucleotide position given by the RS# and genetic position 1565445 and chr9:84846625,
the NTRK2 gene at nucleotide position given by the RS# and genetic position 1387923 and chr9:84870190, and
mutations in linkage disequilibrium with any of the aforementioned nucleotides;
or combinations thereof;
measuring a phenotypic trait in the cybrid cells or animal model that correlates with the presence of said mutational burden and that is not present in cultured cybrid cells or an animal model having genomic DNA originating from a neuronal rho cell line and genomic DNA originating from tissue of a human free of a disorder that is associated with bipolar disorder; and
correlating any change in the phenotypic trait with the effectiveness of the compound.

21. The method according to claim 20 wherein the phenotypic trait comprises:
a blockade of at least one cascade in the Inositol, Serotonergic, Dopaminergic, or Noradrenergic biochemical pathways.

22. The method according to claim 20 wherein the correlating is in accordance with an algorithm drawn from the group consisting essentially of
linear or nonlinear regression algorithms,
linear or nonlinear classification algorithms,
ANOVA,
neural network algorithms,
genetic algorithms,
support vector machines algorithms,
hierarchical analysis or clustering algorithms,
hierarchical algorithms using decision trees,
kernel based machine algorithms including kernel partial least squares algorithms,
kernel matching pursuit algorithms,
kernel fisher discriminate analysis algorithms,
kernel principal components analysis algorithms,
Bayesian probability function algorithms,
Markov Blanket algorithms,
a plurality of algorithms arranged in a committee network, and
forward floating search or backward floating search algorithms.

23. A method for diagnosing bipolar disorder, said method comprising:
determining, in a nucleic acid sequence of a biological sample from a human a mutational burden according to a mutation in genes of the group consisting essentially of
the BNDF gene at nucleotide position given by the RS# and genetic position 2049045 and chr11:27650817.
the IMPA2 gene at nucleotide position given by the RS# and genetic position 971362 and chr18:11970618,
the IMPA2 gene at nucleotide position given by the RS# and genetic position 971363 and chr18:11970460,
the INPP1 gene at nucleotide position given by the RS# and genetic position 972691 and chr2:191053261,
the INPP1 gene at nucleotide position given by the RS# and genetic position 2016037 and chr2:191042763,
the NTRK2 gene at nucleotide position given by the RS# and genetic position 1619120 and chr9:84531750,
the NTRK2 gene at nucleotide position given by the RS# and genetic position 1565445 and chr9:84846625,
the NTRK2 gene at nucleotide position given by the RS# and genetic position 1387923 and chr9:84870190, and
mutations in linkage disequilibrium with any of the aforementioned nucleotides.
or combinations thereof,
one or more nucleotide positions in a sequence region corresponding to a wildtype genomic DNA sequence; and
correlating any determined mutational burden as a diagnosis of bipolar disorder.

24. The method according to claim 23 wherein the correlating is in accordance with an algorithm drawn from the group consisting essentially of:
linear or nonlinear regression algorithms,
linear or nonlinear classification algorithms,
ANOVA,
neural network algorithms,
genetic algorithms,
support vector machines algorithms,
hierarchical analysis or clustering algorithms,
hierarchical algorithms using decision trees,
kernel based machine algorithms including kernel partial least squares algorithms,
kernel matching pursuit algorithms,
kernel fisher discriminate analysis algorithms,
kernel principal components analysis algorithms,
Bayesian probability function algorithms,
Markov Blanket algorithms,
a plurality of algorithms arranged in a committee network, and
forward floating search or backward floating search algorithms.

25. A method according to claim 23 wherein the diagnosis of bipolar disorder is from the determining of mutations occurring within the group of genes consisting essentially of ADRBK2, BNDF, GSK3B, GRK3, IMPA1, IMPA2, INPP1, MARCKS, NTRK2 and/or NR1I2.

26. The method according to claim 23 wherein the type of bipolar disorder diagnosed is treatment-resistant bipolar disorder.

27. The method according to claim 23 wherein the type of bipolar disorder diagnosed is rapid-cycling bipolar disorder.

28. A therapeutic composition comprising:
antisense or small interfering RNA sequences that are specific to mutant genes drawn from the group of genes consisting essentially of
the BNDF gene at nucleotide position given by the RS# and genetic position 2049045 and chr21:27650817.
the IMPA2 gene at nucleotide position given by the RS# and genetic position 971362 and chr18:11970618,
the IMPA2 gene at nucleotide position given by the RS# and genetic position 971363 and chr18:11970460,
the INPP1 gene at nucleotide position given by the RS# and genetic position 972691 and chr2:191053261,
the INPP1 gene at nucleotide position given by the RS# and genetic position 2016037 and chr2:191042763,
the NTRK2 gene at nucleotide position given by the RS# and genetic position 1619120 and chr9:84531750,
the NTRK2 gene at nucleotide position given by the RS# and genetic position 1565445 and chr9:84846625,
the NTRK2 gene at nucleotide position given by the RS# and genetic position 1387923 and chr9:84870190, and
mutations in linkage disequilibrium with any of the aforementioned nucleotides.
or combinations thereof,
or mutant messenger RNA transcribed therefrom;
wherein the antisense or small interfering RNA sequences are adapted to bind to and inhibit transcription or translation of target genes according to the genes having mutational burden without preventing transcription or translation of wild-type genes of the same type.

29. The therapeutic composition of claim 28
wherein the diagnosed bipolar disorder is treated by therapy directed genes selected from the group consisting of: ADRBK2, BNDF, GSK3B, GRK3, IMPA1, IMPA2, INPP1, MARCKS, NTRK2 and/or NR1I2.

30. A kit comprising
devices and reagents for measuring mutational burden in the genes of a patient; and
a computer algorithm consisting of executable computer code residing on a memory medium that, in response to the measured mutational burdens of the patient's genes, determines in and for that patient a diagnosis for mood disorders, or for treatment outcome should the patient be given medication for mood disorders.

31. The kit according to claim 30 for determining patient diagnosis for mood disorders, or for treatment outcome for a medication for mood disorders, wherein the devices and reagents comprise:
a device having reagents at each of a plurality of discrete locations, each reagent and corresponding location configured and arranged to immobilize for detection one of said plurality of subject-derived markers, the device supporting the analysis of mutational content of one or more of the genes drawn from the group consisting of ADRBK2, BNDF, GSK3B, GRK3, IMPA1, IMPA2, INPP1, MARCKS, NTRK2 AND/OR NR1I2;
wherein the computer algorithm is calculating, in consideration of the analyzed mutational burden and additional clinical information, a probability of response to mood disorder medication for the patient, or a diagnosis of a mood disorder of the patient.

32. The kit according to claim 31 when the mutational burden consists essentially of:
a mutation in the BNDF gene at nucleotide position given by the RS# and genetic position 2049045 and chr11:27650817; in the IMPA2 gene at nucleotide position given by the RS# and genetic position 971362 and chr18:11970618; in the IMPA2 gene at nucleotide position given by the RS# and genetic position 971363 and chr18:11970460; in the INPP1 gene at nucleotide position given by the RS# and genetic position 972691 and chr2:191053261; in the INPP1 gene at nucleotide position given by the RS# and genetic position 2016037 and chr2:191042763; in the NTRK2 gene at nucleotide position given by the RS# and genetic position 1619120 and chr9:84531750; in the NTRK2 gene at nucleotide position given by the RS# and genetic position 1565445 and chr9:84846625; in the NTRK2 gene at nucleotide position given by the RS# and genetic position 1387923 and chr9:84870190; mutations in linkage disequilibrium with any of the aforementioned nucleotides; and/or combinations thereof.

33. The kit according to claim 31 when the calculation of diagnostic or probability of response to mood disorder medication is made by a computer algorithm drawn from the group consisting essentially of:
linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; recursive feature elimination or entropy-based recursive feature elimination algorithms; a plurality of algorithms arranged in a committee network; and forward floating search or backward floating search algorithms.

34. The kit according to claim 33 wherein the mood disorder medication is drawn from the group consisting essentially of
depakote,
olanzapine, and
lamotrigine

35. The kit according to claim 31 when the determined patient diagnosis comprises:
risk of developing bipolar disorder;
post-traumatic stress syndrome;
panic disorder; and/or
suicidal ideation.

36. The kit according to claim 31 wherein the device comprises:
an assay selected from the group consisting of a hybridization assay, a sequencing assay, a microsequencing assay and a an enzyme-based mismatch detection assay.
